(19) United States
(12) Patent Application Publication
(10) Pub. No.: US 2024/0287508 A1
(43) Pub. Date: Aug. 29, 2024

(54) MODIFIED FILAMINS AND THEIR USES

(71) Applicant: MEDIZINISCHE UNIVERSITÄT WIEN, Wien (AT)

(72) Inventors: Franz Michael JANTSCH, Wien (AT); Mamta JAIN, Bangalore (IN)

(73) Assignee: MEDIZINISCHE UNIVERSITÄT WIEN, Wien (AT)

(21) Appl. No.: 17/796,968

(22) PCT Filed: Feb. 2, 2021
(86) PCT No.: PCT/EP2021/052418

§ 371 Date: Aug. 2, 2022

(30) Foreign Application Priority Data
Feb. 3, 2020 (EP) 20155180.1

(51) Int. Cl. C12N 15/11; A61K 38/00; A61P 35/00; C07K 14/47

(52) U.S. Cl. CPC C12N 15/111; A61P 35/00; C07K 14/47; A61K 38/00; C12Y 305/04004

(57) ABSTRACT

The present invention relates to the provision of new means and methods for the treatment of proliferative and inflammatory diseases. In particular, the invention relates to a pharmaceutical composition comprising a modified Filamin A encoded by a nucleic acid molecule, wherein the nucleic acid molecule is characterized in that the codon in the wildtype sequence encoding glutamine corresponding to position 2333 of SEQ ID NO: 27 is replaced by a codon encoding arginine and/or a modified Filamin B encoded by a nucleic acid molecule, wherein the nucleic acid molecule is characterized in that the codon in the wildtype sequence encoding glutamine corresponding to position 2327 of SEQ ID NO: 29 is replaced by a codon encoding arginine. The invention further relates to a pharmaceutical composition comprising a nucleic acid molecule encoding a modified Filamin A having an actin-binding domain and an arginine corresponding to the arginine at position 2333 of SEQ ID NO: 1 and/or a nucleic acid molecule encoding a modified Filamin B having an actin-binding domain and an arginine corresponding to the arginine at position 2327 of SEQ ID NO: 5. In addition, the invention provides a pharmaceutical composition comprising an oligonucleotide construct capable of modifying Filamin A RNA, wherein said modification is generated by site-directed RNA editing and comprises the conversion of adenosine corresponding to position 7247 of SEQ ID NO: 7 to inosine and/or capable of modifying Filamin B RNA, wherein said modification is generated by site-directed RNA editing and comprises the conversion of adenosine corresponding to position 7123 of SEQ ID NO: 9 to inosine.

### RELATED APPLICATIONS

The present application is a National Stage entry of International Patent Application No. PCT/EP2021/052418, filed Feb. 2, 2021, which claims priority to European Patent Application No. 20155180.1, filed Feb. 3, 2020.

### SEQUENCE LISTING

The instant application contains a Sequence Listing which has been submitted electronically in ASCII format and is hereby incorporated by reference in its entirety. Said ASCII copy, created on Jan. 17, 2023, is named 028622-0326_Sequence Listing.txt and is 275,050 bytes in size.

The present invention relates to the provision of new means and methods for the treatment of proliferative and inflammatory diseases. In particular, the invention relates to a pharmaceutical composition comprising a modified Filamin A encoded by a nucleic acid molecule, wherein the nucleic acid molecule is characterized in that the codon in the wildtype sequence encoding glutamine corresponding to position 2333 of SEQ ID NO: 27 is replaced by a codon encoding arginine and/or a modified Filamin B encoded by a nucleic acid molecule, wherein the nucleic acid molecule is characterized in that the codon in the wildtype sequence encoding glutamine corresponding to position 2327 of SEQ ID NO: 29 is replaced by a codon encoding arginine. The invention further relates to a pharmaceutical composition comprising a nucleic acid molecule encoding a modified Filamin A having an actin-binding domain and an arginine corresponding to the arginine at position 2333 of SEQ ID NO: 1 and/or a nucleic acid molecule encoding a modified Filamin B having an actin-binding domain and an arginine corresponding to the arginine at position 2327 of SEQ ID NO: 5. In addition, the invention provides a pharmaceutical composition comprising an oligonucleotide construct capable of modifying Filamin A RNA, wherein said modification is generated by site-directed RNA editing and comprises the conversion of adenosine corresponding to position 7247 of SEQ ID NO: 7 to inosine and/or capable of modifying Filamin B RNA, wherein said modification is generated by site-directed RNA editing and comprises the conversion of adenosine corresponding to position 7123 of SEQ ID NO: 9 to inosine.

Cancer is a group of diseases involving abnormal cell proliferation with the potential to invade or spread to other parts of the body. Cancer is a major public health problem worldwide and is the second leading cause of death in the United States. There will be approximately 1,762,450 cancer cases diagnosed in 2019, which is the equivalent of more than 4,800 new cases each day. An estimated 606,880 Americans will die from cancer in 2019, corresponding to almost 1,700 deaths per day. The lifetime probability of being diagnosed with invasive cancer is 39.3% for men and 37.7% for women (Siegel (2019) CA Cancer J Clin 69(1):7-34). Although extensive research relating to cancer has been conducted and pharmaceuticals have been found there is still a significant interest in the development of new cancer treatment strategies.

The mucosa of the gastrointestinal tract is chronically exposed to various antigens found in bacteria and food. Usually, gut homeostasis is maintained by suppressing excessive immune responses to foreign antigens. Failures to regulate the immune response in the gastrointestinal tract can result in Inflammatory bowel disease (IBD). IBD is an idiopathic disorder caused by chronic and excessive inflammation of the gastrointestinal tract, leading to rectal bleeding and weight loss. IBD can be classified into 2 main clinical phenomena: Crohn's disease and colitis (Lee (2018) Intest Res 16(1):26-42). IBDs affect 2.5-3 million people in Europe with a healthcare cost of 4.6-5.6 billion euros/year (Burisch (2013) J Crohns Colitis 7(4):322-37). The incidence of both Crohn's disease and colitis is increasing or stable in virtually every region of the world. Due to the early age of onset and low mortality of IBD patients the prevalence of IBD is expected to increase further. The emergence of IBD in traditionally low-prevalence regions will further contribute to this increase (Molodecky (2011) Gastroenterology 42(1):46-54). Although many investigations have tried to identify novel pathogenic factors associated with IBD that are related to environmental, genetic, microbial, and immune response factors, there is no full understanding of IBD pathogenesis until today. Thus, IBD treatment is far from optimal, and patient outcomes are often unsatisfactory (Lee (2018) loc. cit.). Consequently, there is a significant interest in the development of new IBD treatment strategies.

Accordingly, there is a need for means and methods and treatment options for cancer and inflammatory diseases like colitis.

Thus, the technical problem underlying the present invention is the provision of means and methods to treat proliferative and inflammatory diseases. The technical problem is solved and the above mentioned needs are addressed by the provision of the embodiments characterized in the claims and as provided herein below.

Accordingly, the present invention relates to a pharmaceutical composition comprising a modified Filamin A encoded by a nucleic acid molecule, wherein the nucleic acid molecule is characterized in that the codon in the wildtype sequence encoding glutamine corresponding to position 2333 of SEQ ID NO: 27 is replaced by a codon encoding arginine and/or a modified Filamin B encoded by a nucleic acid molecule, wherein the nucleic acid molecule is characterized in that the codon in the wildtype sequence encoding glutamine corresponding to position 2327 of SEQ ID NO: 29 is replaced by a codon encoding arginine.

SEQ ID NO: 27 corresponds to the sequence as annotated under the NCBI Reference Sequence NP_001447.2. SEQ ID NO: 29 corresponds to the sequence as annotated under the NCBI Reference Sequence NP_001157789.1.

The term “Filamin A” means, in accordance with the present invention, an actin-crosslinking protein or a polypeptide, which comprises an actin-binding domain at its amino-terminus and a dimerization domain at the carboxyl-terminus (Nakamura (2011) Cell Adh Migr 5:160-169). The protein is built of 24 immunoglobulin (Ig)-like domains. Ig domains 1 through 15 build a stiff region termed rod 1, while Ig repeats 16 through 23 build rod 2. The Ig repeat 24 can form a homo-dimer with another Ig repeat of another filamin A molecule. However, the Ig 24 repeat of a Filamin A molecule can also form a heterodimer with an Ig repeat 24 of a Filamin B molecule (Sheen (2002) Hum Mol Genet 11:2845-2854). The term “Filamin B” means, in accordance with the present invention, an actin-crosslinking protein or a polypeptide, which comprises an actin-binding domain at its amino-terminus and a dimerization domain at the carboxyl-terminus (Nakamura (2011) loc. cit.). The protein is built of 24 immunoglobulin (Ig)-like domains. The Ig repeat 24 can form a homo-dimer with another Ig repeat of another Filamin B molecule. However, the Ig 24 repeat of a Filamin B molecule can also form a heterodimer with an Ig repeat 24 of a Filamin A molecule (Sheen (2002) loc. cit.).

The term “polypeptide” means, in accordance with the present invention a protein or a polypeptide which encompasses amino acid chains of a given length, wherein the amino acid residues are linked by covalent peptide bonds. However, peptidomimetics of such proteins/polypeptides wherein amino acid(s) and/or peptide bond(s) have been replaced by functional analogs are also encompassed by the invention.

The meaning of the term “nucleic acid sequence(s)/molecule(s)” are well known in the art, are used accordingly in context of the present invention and described in detail further below.

The term “modified Filamin A” as used herein means that the glutamine residue corresponding to the glutamine residue at position 2333 of the wildtype sequence of isoform 1 of human Filamin A (SEQ ID NO:27) is replaced by an arginine residue.

The term “the codon in the wildtype sequence encoding glutamine corresponding to position 2333 of SEQ ID NO: 27 is replaced by a codon encoding arginine” means, according to this invention, a codon encoding a specific glutamine residue in a known wildtype sequence of Filamin A is replaced by a codon encoding arginine. The wildtype amino acid sequence of the isoform 1 of human Filamin A is well known in the art and, inter alia, shown in SEQ ID NO: 27. Said glutamine of position 2333 of isoform 1 of human Filamin A corresponds, e.g., to the glutamine of position 2341 of isoform 2 of human Filamin A (SEQ ID NO: 28; SEQ ID NO: 28 corresponds to the sequence as annotated under the NCBI Reference Sequence NP_001104026.1.), the glutamine of position 2333 of mouse Filamin A isoform 1 (NCBI Reference Sequence NP_034357.2), the glutamine of position 2333 of rat Filamin A (NCBI Reference Sequence NP_001128071.1), the glutamine of position 2341 of isoform 1 of chimpanzee Filamin A (NCBI reference sequence XP_016803150.1)

In context of the present invention “modified” and “edited” may be used interchangeably. The term “the codon in the wildtype sequence encoding glutamine corresponding to position 2327 of SEQ ID NO: 29 is replaced by a codon encoding arginine.” means, according to this invention, a codon encoding a specific glutamine in a known wildtype sequence of Filamin B is replaced by a codon encoding arginine. The wildtype amino acid sequence of the of human Filamin B is well known in the art and, inter alia, shown in SEQ ID NO: 29. Said glutamine of position 2327 of human Filamin B corresponds, e.g., to the glutamine of position 2272 of mouse Filamin B isoform X1 (NCBI reference sequence XP_006518113.1), to the glutamine of position 2285 of rat Filamin B isoform X2 (NCBI reference sequence XP_006251843.1), or to glutamine of position 2285 of chimpanzee Filamin B isoform X2 (NCBI reference sequence XP_008962280.1).

In order to determine whether a nucleotide residue/position or an amino acid residue/position in a given nucleotide sequence or amino acid sequence, respectively, corresponds to a certain position compared to another nucleotide sequence or amino acid sequence, respectively, the skilled person can use means and methods well known in the art, e.g., alignments, either manually or by using computer programs such as those mentioned herein. For example, BLAST 2.0 can be used to search for local sequence alignments. Similarly, alignments may also be based on the CLUSTALW computer program (Thompson (1994) Nucl Acids Res. 2: 4673-4680) or CLUSTAL Omega (Sievers (2014) Curr Protoc Bioinformatics 48: 3.13.1-3.13.16).

In context of the present invention it has been surprisingly found that Filamin A RNA editing has an effect on neo-vascularization in tumor angiogenesis. Transgenic mice mimicking the modified state, i.e., producing exclusively modified (edited) Filamin A and mimicking the unmodified state, i.e., producing exclusively unmodified (unedited) Filamin A were injected with B16-F10 melanoma cells to grow xenograft tumors. Surprisingly and unexpectedly the xenograft tumors grown in mice producing unmodified Filamin A grow much bigger (in both weight and volume) when compared to tumors injected in mice producing modified Filamin A demonstrating that Filamin A modification can critically regulate tumor growth. Further, blood vessel density in tumors grown in unmodified Filamin A mice is significantly increased when compared to the smaller tumors observed in modified Filamin A mice. These results show for the first time that the glutamine to arginine exchange in Filamin A naturally occurring due to an adenosine to inosine (A-to-I) RNA editing event can regulate cell migration, new vessel formation and thus affect tumor growth. Thus, it is envisaged herein that modified Filamin A and/or modified Filamin B may be used for the treatment of cancer.

As depicted in the appended examples it has been surprisingly found that that Filamin A RNA editing has a role in in gastrointestinal integrity and inflammatory bowel diseases. Using dextran sodium sulfate (DSS) to induce colitis in mice, it is shown that mice producing modified Filamin A are protected against induced colitis when compared to wild type, or unmodified Filamin A mice. Colitis manifestation in mice producing unmodified Filamin A was much stronger in the distal part of the colon where Filamin A RNA editing tends to be higher than in the proximal part of the colon. Thus, in context of the present invention it is shown for the first time that the glutamine to arginine exchange in Filamin A naturally occurring due to an A-to-I RNA editing event can regulate the progression of induced colitis. Thus, it is envisaged herein that modified Filamin A and/or modified Filamin B may be used to treat colon inflammation, colitis and Crohn's disease, the two major IBDs affecting human health.

Furthermore, is has been surprisingly found in context of the present invention that Filamin A editing has an effect on wound healing. A wound healing assay was performed by inserting a 4 mm diameter whole on the shaved back of a narcotized mouse. Subsequently, the wound closure was measured for 10 days using a caliper. Using this method, a delay in wound healing was observed in mice producing unmodified Filamin A compared to wild-type mice. Accordingly, it is also envisaged herein that modified Filamin A and/or modified Filamin B may be used to enhance wound healing, i.e., to treat skin lesions or mucosa lesions.

Thousands of RNA modifications known to date can control the fate and function of RNAs and therefore affect gene expression at the transcript level. The fact that some RNA modifications can be introduced (written), recognized (sensed) and removed (erased) has keyed the term “Epitranscriptomics” (Witkin (2015) Cancer Biol Ther 16: 21-27).

A-to-I RNA editing is one of the most abundant post-transcriptional RNA modifications in metazoa. A-to-I editing occurs by a deamination reaction catalyzed by ADARs. ADARs are multi-domain proteins, comprising a recognition domain and a catalytic domain. The recognition domain recognizes a specific double-stranded RNA (dsRNA) sequence and/or conformation, whereas the catalytic domain converts an adenosine into an inosine in a nearby, more or less predefined, position in the target RNA, by deamination of the nucleobase. Out of the three mammalian ADAR (ADAR1, ADAR2, ADAR3) enzymes known, ADAR1 and ADAR2 are catalytically active (Bass (2002) Annu Rev Biochem 71:817-46 Gerber (2001) Trends Biochem Sci 26(6):376-84; Nishikura (2016) Nat Rev Mol Cell Biol 17(2):83-96; Xu (2018) Curr Opin Genet Dev 48:51-56). As cellular machineries interpret inosines as guanosines, editing events can affect secondary structure and stability of RNA, alter splicing and change miRNAs target specificity (Agrawal (2005) RNA 11(5):563-6; Kawahara (2008) Nucleic Acids Res 36(16):5270-80; Kawahara (2007) Science 315(5815):1137-40; Licht (2019) Nucleic Acids Res 47(1):3-14). An A-to-I conversion in coding mRNAs can lead to an amino acid exchange in the encoded protein and thus to recoding of genomically encoded information. ADAR1 knock out mice die at E12.5 due to hyperinflammation as a result of an increased interferon response (Hartner (2004) J Biol Chem 279(6):4894-902; Hartner (2009) Nat Immunol 10(1):109-15). ADAR2 knockout mice die within three weeks after birth due to seizures. However, this lethal phenotype can be rescued by introduction of a pre-edited glutamate receptor subunit, Gria2 (Brusa (1995) Science 270(5242):1677-80; Higuchi (2000) Nature 406(6791):78-81).

A-to-I RNA editing has been shown to regulate several diseases including cancer. RNA seq data from The cancer Genome Atlas (TCGA) and other high-throughput studies show that many tumors show increased editing (Paz-Yaacov (2015) Cell Rep 13(2):267-76; Han (2015) Cancer Cell 28(4):515-528). An increase in editing is frequently associated with elevated ADAR1 levels. Several editing induced amino acid exchanges have been shown to play an important role in tumor progression and metastasis. One classical example is the role of Azin1 editing in hepatocellular carcinoma (Chen (2013) Nat Med 19(2):209-16). The edited version of Azin1 translocates into the nucleus and stimulates cell proliferation and favors tumor growth (Chen (2013) loc. cit.). RHOQ editing is increased in colorectal cancer and correlates with increased invasion and actin cytoskeleton reorganization (Han J Exp Med 211(4):613-21). RNA editing of SLC22A3 is also positively correlated with the progression of ESCC (Esophageal squamous cell carcinoma) (Fu (2017) Proc Natl Acad Sci USA 114(23):E4631-E4640). However, A-to-I RNA editing in RNAs other than Filamin A or Filamin B RNA has also been linked with or shown to suppress tumor growth (Chan (2016) Gastroenterology 151(4):637-650 e10; Chen (2017) Int J Oncol 50(2):622-630; Galeano (2013) Oncogene 32(8):998-1009; Gumireddy (2016) Nat Commun 7:10715). Overall, tumor onset and progression may be regulated by the interplay of hyper and hypo-edited targets. More recently, ADAR1 has been shown to control immune checkpoint in cancer (Ishizuka (2019) Nature 565(7737):43-48). Loss of ADAR1 reduces hyperedited interferon-inducible RNAs thus activating MDA5 and PKR to activate an immune response. ADAR1 deletion leads to smaller tumors as this helps to overcome resistance to immune checkpoint blockade (Ishizuka (2019) loc. cit.).

Filamins are actin crosslinking proteins that are built of 24 Ig-like domains and can form homo- and heterodimers (Razinia, (2012) Annu Rev Biophys 41, 227-246; Zhou, (2010) Trends Cell Biol 20, 113-123.). It is known that Filamin A and Filamin B heterodimerize and that the homologous amino acids in the orthologous proteins are edited (Razinia, (2012) Annu Rev Biophys 41:227-246; Czermak (2018) RNA Biol 15:877-885). The pre-mRNAs encoding Filamin A and Filamin B can be targeted by ADAR2 in a region corresponding to exon 42. This editing event can lead to a Q (glutamine) to R (arginine) exchange at position 2341 in mouse Filamin A (uniprot accession no. Q8BTM8) and at position 2296 in mouse Filamin B (uniprot accession no. Q80X90) (Levanon (2005) Nucleic Acids Res 33(4):1162-8). Filamin A and Filamin B editing are spatially and temporally regulated. Generally, Filamin A and Filamin B RNA editing increases with age. Filamin A RNA editing reaches over 80% in the arterial system, stomach and large intestine (Jain (2018) EMBO J 37(19); Stulic (2013) RNA Biol 10(10):1611-7). Filamin B RNA editing, in contrast is highest in bone, cartilage and fat (Czermak (2018) RNA Biol 15:877-885). Besides actin-crosslinking, Filamins can act as mechanosensors (Stossel (2001) Nat Rev Mol Cell Biol 2(2):138-45). The C-terminus of Filamins acts as a platform to bind over 100 proteins including signaling molecules, transcription factors, receptors and other cytoskeletal proteins (Nakamura (2011) Cell Adh Migr 5(2):160-9; Popowicz (2006) Trends Biochem Sci 31(7):411-9). The editing-induced amino acid exchange is located within this region spanning a hot spot for protein-protein interactions. Thus, the editing status of Filamin A and Filamin B could regulate the interactome of Filamin A and Filamin B proteins, their mechanosensing properties and their turnover. Recently, we showed that Filamin A RNA editing plays an important role in regulating hypertension and cardiac health. Mice lacking Filamin A RNA editing show increased smooth muscle contraction, leading to elevated blood pressure and cardiac remodeling. These data highlight the importance of A-to-I RNA editing in the cardiovascular system (Jain (2018) loc. cit.).

Mutations in human Filamin A lead to neuronal and intestinal diseases (Gargiulo (2007) Am J Hum Genet 80(4):751-8; Hehr (2006) J Med Genet 43(6):541-4). Filamin A deficient mice are embryonic lethal showing severe cardiovascular defects and irregular vascular patterning (Feng (2006) Proc Natl Acad Sci USA 103(52):19836-41). Deletion of Filamin B in mice leads to skeletal malformations (Zhou (2007) Trends Cardiovasc Med 17(7):222-9). Filamin A is known to play a dual role in tumorigenesis. Filamin A can get proteolytically cleaved leading to differential subcellular localization of the resulting fragments. Full-length, cytoplasmic Filamin A could favor tumor progression whereas nuclear Filamin A may inhibit tumorigenesis (Savoy (2013) Endocr Relat Cancer 20(6):R341-56; Shao (2016) Pathol Oncol Res 22(2):245-52). An endothelial specific knock-out of Filamin A results in altered cardiac remodeling, reduced capillary formation and dysregulated endothelial signaling upon induced infarction (Bandaru (2015) Cardiovasc Res 105(2):151-9). Moreover, siRNA mediated knockdown of Filamin A in human endothelial cells (HUVECs) leads to reduced tube formation and cell migration (Bandaru (2015) loc. cit.).

The invention further relates to a pharmaceutical composition wherein the modified Filamin A has an actin-binding domain and an arginine corresponding to the arginine at position 2333 of SEQ ID NO: 1 and is encoded by a nucleic acid molecule selected from the group consisting of (i) a nucleic acid molecule encoding a polypeptide comprising an amino sequence as depicted in SEQ ID NOs: 1 or 3;
(ii) a nucleic acid molecule comprising a nucleotide sequence as depicted in SEQ ID NOs: 2 or 4;
(iii) a nucleic acid molecule hybridizing under stringent conditions to the complementary strand of the nucleic acid molecule of (i) to (ii);
(iv) a nucleic acid molecule having a nucleotide sequence with at least 60% sequence identity to the nucleotide sequence of a nucleic acid molecule of any one of (i) to (iii); and
(v) a nucleic acid molecule comprising a nucleotide sequence which is degenerate as a result of the genetic code to the nucleotide sequence of a nucleic acid molecule of any one of (i) to (iv); and/or wherein the modified Filamin B has an actin-binding domain and an arginine corresponding to the arginine at position 2327 of SEQ ID NO: 5 and is encoded by a nucleic acid molecule selected from the group consisting of
(a) a nucleic acid molecule encoding a polypeptide comprising an amino sequence as depicted in SEQ ID NO: 5;
(b) a nucleic acid molecule comprising a nucleotide sequence as depicted in SEQ ID NO: 6;
(c) a nucleic acid molecule hybridizing under stringent conditions to the complementary strand of a nucleic acid molecule of (a) or (b);
(d) a nucleic acid molecule having a nucleotide sequence with at least 60% sequence identity to the nucleotide sequence of a nucleic acid molecule of any one of (a) to (c); and
(e) a nucleic acid molecule comprising a nucleotide sequence which is degenerate as a result of the genetic code to the nucleotide sequence of a nucleic acid molecule of any one of (a) to (d).

In a preferred embodiment the modified Filamin A has an actin-binding domain and an arginine corresponding to the arginine at position 2333 of SEQ ID NO: 1 and an amino acid sequence selected from the group consisting of SEQ ID NOs: 1 or 3 or an amino acid sequence having at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said amino acid sequences. The skilled person understands that the arginine residue corresponding to position 2333 of SEQ ID NO: 1 or to position 2341 SEQ ID NO: 3 is crucial for the herein described effect of modified Filamin A.

The skilled person is well aware that also other isoforms of Filamin A may be used as a medicament as long as the glutamine residue corresponding to amino acid position 2333 of SEQ ID NO: 27 is substituted with an arginine residue.

In a preferred embodiment the modified Filamin B as described herein has an actin-binding domain and an arginine corresponding to the arginine at position 2327 of SEQ ID NO: 5 and an amino acid sequence selected from the group consisting of SEQ ID NO: 5 or an amino acid sequence having at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said amino acid sequence. The skilled person understands that the arginine residue corresponding to position 2327 of SEQ ID NO: 5 is crucial for the herein described effect of modified Filamin B.

The skilled person is well aware that also other isoforms of Filamin B may be used as a medicament as long as the glutamine residue corresponding to amino acid position 2327 of SEQ ID NO: 29 is substituted with an arginine residue.

It is noted that the pharmaceutical compositions of the invention may also comprise fragments of the polypeptides described herein. Without being bound by scientific theory it is envisaged herein that pharmaceutical compositions of the present invention comprise Filamin A fragments comprising the arginine corresponding to the arginine at position 2333 of SEQ ID NO: 1 and/or Filamin B fragments comprising arginine corresponding to the arginine at position 2327 of SEQ ID NO: 5.

The present invention relates to a modified Filamin A and/or modified Filamin B as described herein for use as a medicament. Accordingly, the present invention relates to a modified Filamin A and/or modified Filamin B as described herein for use in the treatment of a disease. Likewise, the present invention relates to a modified Filamin A and/or modified Filamin B as described herein for use in the preparation of a medicament for the treatment of a disease. Said disease may be a proliferative or inflammatory disease. Said disease may also be a skin lesion, mucosa lesion or another lesion of a structure of the body.

It is also envisaged in the present invention that the modified Filamin A and/or modified Filamin B in the pharmaceutical composition is generated via RNA editing. Accordingly, the present invention relates to a pharmaceutical composition, wherein the modified Filamin A and/or modified Filamin B is generated via RNA editing.

The term “RNA Editing” as used herein means that the base of a nucleotide of a RNA molecule is modified. Chemical groups may be added or removed from the base. Preferably, a chemical group is removed from the base. Preferably, an amino group is removed from the base. Preferably, an amino group is removed from an adenine base. Most preferably, an adenine base is converted to a hypoxanthine base by deamination. Consequently, an adenosine is converted to inosine by deamination.

Preferably, RNA editing is site-specific RNA editing. The term “site-specific editing” as used herein means that a specific nucleotide in a target RNA molecule is edited as described above. Therefore, the present invention relates to a pharmaceutical composition, wherein the modified Filamin A and/or modified Filamin B is generated via site-specific RNA editing The editing may be performed by an RNA editing entity. Thus, the present invention relates to a pharmaceutical composition, wherein the modified Filamin A and/or modified Filamin B is generated via site-specific RNA editing performed by an RNA editing entity. The RNA editing entity may be an enzyme. Thus, the present invention relates to a pharmaceutical composition, wherein the modified Filamin A and/or modified Filamin B is generated via site-specific RNA editing performed by an enzyme. Accordingly, the deamination of adenosine to inosine may be performed by an enzyme. Preferably, the enzyme is an ADAR. Accordingly, the present invention relates to a pharmaceutical composition, wherein the modified Filamin A and/or modified Filamin B is generated via site-specific RNA editing performed by ADAR. More preferably, the enzyme is ADAR1 or ADAR2, which means that the conversion from adenosine to inosine by deamination is performed by ADAR1 or ADAR2. Accordingly, the present invention relates to a pharmaceutical composition, wherein the modified Filamin A and/or modified Filamin B is generated via site-specific RNA editing performed by ADAR1 and/or ADAR2.

The RNA editing as described herein can take place in vivo, ex vivo and/or in vitro. The term “in vitro” does not exclude cellular systems.

Without being bound by scientific theory it is also envisaged in context of the present invention that cells or tissue are/is removed from a patient, RNA editing as described herein is induced in said cells or tissue and said treated cells or tissue are/is reintroduced in the patient.

Although clear for the skilled person it is emphasized that the modified Filamin A and/or Filamin B can be delivered in form of a nucleic acid molecule encoding said modified Filamin A and/or Filamin B. Accordingly, the present invention also relates to a pharmaceutical composition comprising a nucleic acid molecule encoding a modified Filamin A, wherein said nucleic acid molecule is selected from the group consisting of:
(i) a nucleic acid molecule comprising the nucleotide sequence as depicted in SEQ ID NOs: 2 and 4;
(ii) a nucleic acid molecule hybridizing under stringent conditions to the complementary strand of a nucleic acid molecule of (i);
(iii) a nucleic acid molecule having a nucleotide sequence with at least 60% sequence identity to the nucleotide sequence of a nucleic acid molecule of (i) or (ii); and
(iv) a nucleic acid molecule comprising a nucleotide sequence which is degenerate as a result of the genetic code to the nucleotide sequence of a nucleic acid molecule of any one of (i) to (iii);
wherein said modified Filamin A has an actin-binding domain and an arginine corresponding to the arginine at position 2333 of SEQ ID NO: 1;
and/or comprising a modified Filamin B, wherein said nucleic acid molecule is selected from the group consisting of:
(a) a nucleic acid molecule comprising the nucleotide sequence as depicted in SEQ ID NO: 6;
(b) a nucleic acid molecule hybridizing under stringent conditions to the complementary strand of a nucleic acid molecule of (a);
(c) a nucleic acid molecule having a nucleotide sequence with at least 60% sequence identity to the nucleotide sequence of a nucleic acid molecule of (a) or (b); and
(d) a nucleic acid molecule comprising a nucleotide sequence which is degenerate as a result of the genetic code to the nucleotide sequence of a nucleic acid molecule of any one of (a) to (c);
wherein said modified Filamin B has an actin-binding domain and an arginine corresponding to the arginine at position 2327 of SEQ ID NO: 5.

In a preferred embodiment the modified Filamin A as described herein has an actin-binding domain and an arginine corresponding to the arginine at position 2333 of SEQ ID NO: 1 and is encoded by a nucleic acid molecule having a nucleotide sequence as depicted in SEQ ID NOs: 2 and 4 or a nucleotide sequence having at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said nucleotide sequences. The amino acid sequence of SEQ ID NO: 1 is encoded by the nucleic acid depicted in SEQ ID NO: 2. The amino acid sequence of SEQ ID NO: 3 is encoded by the nucleic acid depicted in SEQ ID NO: 4.

In a preferred embodiment the modified Filamin B as described herein has an actin-binding domain and an arginine corresponding to the arginine at position 2327 and is encoded by a nucleic acid molecule having a nucleotide sequence as depicted in SEQ ID NO: 6 or a nucleotide sequence having at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said nucleotide sequence.

The present invention relates to a nucleic acid molecule as described herein for use as a medicament. Accordingly, the present invention relates to a nucleic acid molecule as described herein for use in the treatment of a disease. Likewise, the present invention relates to a nucleic acid molecule as described herein for use in the preparation of a medicament for the treatment of a disease. Said disease may be a proliferative or inflammatory disease. Said disease may also be a skin lesion, mucosa lesion or another lesion of a structure of the body.

The meaning of the term “nucleic acid sequence(s)/molecule(s)” are well known in the art and are used accordingly in context of the present invention. For example, “nucleic acid sequence(s)/molecule(s)” as used herein refer(s) to all forms of naturally occurring or recombinantly generated types of nucleic acids and/or nucleic acid sequences/molecules as well as to chemically synthesized nucleic acid sequences/molecules. This term also encompasses nucleic acid analogues and nucleic acid derivatives. The term “nucleic acid sequence(s)/molecule(s)” can refer to deoxyribonucleic acid (DNA) and ribonucleic acid (RNA). The “nucleic acid sequence(s)/molecule(s)” may be made by synthetic chemical methodology known to one of ordinary skill in the art, or by the use of recombinant technology, or may be isolated from natural sources, or by a combination thereof. The DNA and RNA may optionally comprise unnatural nucleotides and may be single or double stranded. “Nucleic acid sequence(s)/molecule(s)” also refers to sense and anti-sense DNA and RNA, that is, a nucleotide sequence which is complementary to a specific sequence of nucleotides in DNA and/or RNA. Furthermore, the term “nucleic acid sequence(s)/molecule(s)” may refer to DNA or RNA or hybrids thereof or any modification thereof that is known in the state of the art (see, e.g., U.S. Pat. Nos. 5,525,711, 4,711,955, 5,792,608 or EP 302175 for examples of modifications). The nucleic acid molecule(s) may be single- or double-stranded, linear or circular, natural or synthetic, and without any size limitation. For instance, the nucleic acid molecule(s) may be genomic DNA, cDNA, mRNA, antisense RNA, or a DNA encoding such RNAs or chimeroplasts (Colestrauss, Science (1996), 1386-1389). Said nucleic acid molecule(s) may be in the form of a plasmid or of viral DNA or RNA.

The terms “encode” or “encoding” are used interchangeably with the terms “encode for” or “encoding for”, respectively. These terms mean that a nucleic acid sequence may serve as template for production of the “encoded amino acid sequence” according to the known rules of the genetic code. Depending on the organism the genetic code may be adapted.

The nucleic acid molecule provided herein may be an open reading frame; i.e., a continuous stretch of codons capable of being translated into an amino acid sequence that starts with a translation start codon (including alternative start codons known in the art) and ends with a translation stop codon. The term “open reading frame” is interchangeably used with “coding sequence” herein.

Accordingly, the nucleic acid molecule as described herein may comprise further features required to express the nucleic acid sequence encoding the polypeptide of the invention in a host cell. For instance, the nucleic acid sequence may be operably linked to a promoter sequence. The nucleic acid molecule as described herein may further comprise regulatory sequences. Regulatory sequences are well known to those skilled in the art and include but are not limited to regulatory sequences ensuring the initiation of transcription, internal ribosomal entry sites (IRES) (Owens (2001) Proc. Natl. Acad. Sci. U.S.A. 98:1471-1476), regulatory elements ensuring termination of transcription or stabilization of the transcript, enhancers and insulators.

Ligation of the coding sequences to transcriptional regulatory elements and/or to other amino acid encoding sequences can be carried out using established methods, such as restriction digests, ligations and molecular cloning. The person skilled in the art is familiar with the preparation and the use of nucleic acid (see, e.g., Sambrook and Russel (2001) Molecular Cloning, A Laboratory Manual Cold Spring Harbor Laboratory, N.Y.).

The present invention further relates to a pharmaceutical composition comprising a vector comprising (a) nucleic acid molecule(s) encoding modified Filamin A and/or Filamin B as described herein. Many suitable vectors are known to those skilled in molecular biology, the choice of which depends on the desired function. Non-limiting examples of vectors include plasmids, cosmids, viruses, bacteriophages and other vectors used conventionally in, e.g., genetic engineering. Methods which are well known to those skilled in the art can be used to construct various plasmids and vectors (see, e.g., Ausubel (1989) Current Protocols in Molecular Biology, Green Publishing Associates and Wiley Interscience, N.Y.).

The vector preferably comprises a promoter being operably linked to the nucleic acid. “Operably linked” means that the promoter is positioned so that it drives the expression of the nucleic acid. Preferably, the vector of the invention is an expression vector. An expression vector according to this invention is capable of directing the replication and the expression of the nucleic acid molecule as described herein in a host or host cell and, accordingly, provides for the expression of the polypeptide as described herein encoded thereby in the selected host or host cell. Expression comprises transcription of the nucleic acid molecule, for example into a translatable mRNA and translation into a polypeptide.

The nucleic acid molecules and/or vectors of the invention can be designed for introduction into cells by, e.g. chemical based methods (polyethylenimine, calcium phosphate, liposomes, DEAE-dextrane, nucleofection, lipid nanoparticles, lipofectamine, preferably lipofectamine 3000), non-chemical methods (electroporation, sonoporation, optical transfection, gene electrotransfer, hydrodynamic delivery or naturally occurring transformation upon contacting cells with the nucleic acid molecule of the invention), particle-based methods (gene gun, magnetofection, impalefection), phage vector-based methods and viral methods. For example, expression vectors derived from viruses such as retroviruses, vaccinia virus, adenovirus, adeno-associated virus, herpes viruses, Semliki Forest Virus or bovine papilloma virus, may be used for delivery of the nucleic acid molecules into targeted cell population.

Delivery to epithelial cells in the large intestine may be performed by enema of the herein described nucleic acids and vectors. For the administration of nucleic acids and/or vectors by enema the nucleic acids and/or vectors may be complexed with lipofectamine 3000 or packaged into lipid nanoparticles.

Delivery to endothelial cells can be performed by intravenous delivery via the blood stream of the herein described nucleic acids and vectors complexed with lipofectamine 3000 or packaged into lipid nanoparticles. Delivery and uptake of nucleic acids and/or vectors described herein may further be facilitated by coupling to antibodies (e.g. anti CD31), small molecules or peptides binding to endothelial cells.

The present invention relates to a vector as described herein for use as a medicament. Accordingly, the present invention relates to a vector as described herein for use in the treatment of a disease. Likewise, the present invention relates to a vector as described herein for use in the preparation of a medicament for the treatment of a disease. Said disease may be a proliferative or inflammatory disease. Said disease may also be a skin lesion, mucosa lesion or another lesion of a structure of the body.

It is further envisaged herein that diseases are treated by enhancing the editing of endogenous Filamin A and/or Filamin B RNA. Adenosines targeted by ADARs occur within structures recognized by ADARs. The RNA binding domain of ADARs recognize A-form helices formed by duplex RNA (double stranded RNA) and imperfect structures with bulges, hairpins and mismatches (Montiel Gonzalez (2019) Methods 156:16-24). One possibility to enhance the editing of adenosines naturally targeted by ADARs is to artificially induce the structures which are recognized by the ADARs (Qu (2019) Nat Biotechnol 37:1059-1069). One strategy may be to provide an oligonucleotide construct (ONC) that binds to the RNA forming a double stranded RNA (dsRNA) surrounding the target adenosine, preferably with a mismatch directly under the target adenosine. The terms “ONC” and “oligonucleotide” may be used synonymously herein. “Target adenosine” as used herein refers to the adenosine, which is supposed to be edited. The dsRNA is recognized by endogenous ADAR and the target adenosine is edited.

In other words, it is envisaged that endogenous ADAR proteins can be targeted to pre-mRNAs or mRNAs for site-directed RNA editing. The editing event can take place in the nucleus or in the cytoplasm. Here, an oligonucleotide construct (ONC) complementary or partially complementary to the RNA binds to the RNA and provides structures upon base pairing with the RNA recognized by endogenous ADARs. The endogenous ADARs bind to said structure formed by the RNA and the ONC and performs the RNA editing. The RNA to which the ONC binds is called the “target RNA” and the sequence in said target RNA to which the ONC is complementary or partially complementary is called the “target sequence”. The term “oligonucleotide construct” is used herein in the broadest sense and refers to any molecule that is able to trigger the site-directed editing of a nucleotide in a target RNA. The degree of complementarity of the ONC to the target sequence is preferably such that the ONC can form a stable hybrid with the target sequence in the RNA molecule under physiological conditions.

Accordingly, the present invention relates to pharmaceutical composition comprising an ONC capable of modifying Filamin A RNA, wherein said modification is generated by site-directed RNA editing and comprises the conversion of adenosine corresponding to position 7247 of SEQ ID NO: 7 to inosine and/or capable of modifying Filamin B RNA, wherein said modification is generated by site-directed RNA editing and comprises the conversion of adenosine corresponding to position 7123 of SEQ ID NO: 9 to inosine.

In addition, the present invention relates to a pharmaceutical composition comprising an ONC capable of modifying Filamin A RNA, wherein the oligonucleotide construct is complementary or partially complementary to parts of sequences SEQ ID NO: 7 or SEQ ID NO: 8 and provides structures upon base pairing with the target sequence that allow recruiting of an RNA editing entity naturally present in the cell or provided together with the oligonucleotide construct and capable of performing the editing of the nucleotide.

Furthermore, the present invention relates to a pharmaceutical composition comprising an ONC capable of modifying Filamin B RNA, wherein the oligonucleotide construct is complementary or partially complementary to parts of the sequence depicted in SEQ ID NO: 9. It is noted that the invention also provides a pharmaceutical composition comprising both ONCs as described directly above.

Preferably, the ONCs in context of the invention bind to Filamin A RNA around the naturally edited adenosine and form dsRNA with Filamin A RNA that is recognized by endogenous ADAR. Said ONCs may bind to a sequence close to position 7247 of SEQ ID NO: 7 or 7267 of SEQ ID NO: 8. Preferably, the ONC binds to a sequence of SEQ ID NO: 7 or SEQ ID NO: 8 including position 7247 of SEQ ID NO: 7 or 7267 of SEQ ID NO: 8. The ONC may be perfectly complementary to the target sequence. However, the ONC may also contain several mismatches as long as the ONC binds to the target sequence under physiological conditions. Preferably, the ONC comprises a mismatch directly vis-à-vis the target adenosine. In other words, it is preferred that the ONC binds to a sequence of SEQ ID NO: 7 or SEQ ID NO: 8 including position 7247 of SEQ ID NO: 7 or 7267 of SEQ ID NO: 8 and the nucleotide in the ONC facing the target adenosine does not contain thymine or uracil. More preferably, the ONC binds to a sequence of SEQ ID NO: 7 or SEQ ID NO: 8 including position 7247 of SEQ ID NO: 7 or 7267 of SEQ ID NO: 8 and is perfectly complementary to said sequence except for one mismatch directly vis-à-vis the target adenosine.

The sequences of the ONCs binding to Filamin A RNA may be selected from the group consisting of the sequences as depicted in SEQ ID NOs: 10 to 13 and 56 and sequences having at least 20%, preferably at least 25%, even more preferably at least 30%, even more preferably at least 40%, even more preferably at least 50%, even more preferably at least 60%, even more preferably at least 70%, even more preferably at least 80%, even more preferably at least 90%, even more preferably at least 95%, even more preferably at least 98% and most preferably at least 99% sequence identity to said sequences. ONCs as described here in SEQ ID Nos:10 to 13 or 56 (and the herein described related sequences having at least 20% or more sequence identity) are “human” sequences and are, therefore, potentially useful in the herein described medical uses/interventions.

ONCs may have undesired off-target effects, i.e. ONCs may induce editing of off-target adenosines. The skilled person is readily in the position to take measures to reduce said off-target effects, e.g. by adapting the sequence or chemical modification of the ONC. Such modifications may occur at the nucleobase, the ribose, the deoxyribose, or the phosphate backbone.

The position in mouse Filamin A RNA (NCBI reference sequence XM_036161857.1; SEQ ID NO:50) corresponding to position 7247 in human Filamin A RNA (SEQ ID NO: 7) is position 7245. An exemplary ONC that is able to bind mouse Filamin A RNA (SEQ ID NO: 50) and is able to induce editing of the target adenosine at position 7245 has the sequence SEQ ID NO: 51 and may have the modifications as shown below.
5′-
mA*mA*mA*GmA*rUmGrCrUrGrGrCrUrGmGrUrUmGrArCrCrUmUrUrArAmCrCrCmUr GrArCrUCCCGrArArGrArCrUmArGmA*mA*mA*mC-3′
where
A is desoxyadenosine,
G is desoxyguanosine,
C is desoxycytosine,
T is desoxythymidine,
stands for phosphothioate linkage,
m stands for 2′ O-methyl ribose,
rU is uridine,
rA is adenosine,
rC is cytidine, and
rG is guanosine.

The ONC corresponding to the ONC with SEQ ID NO: 50 that targets human Filamin A mRNA has the sequence as shown in SEQ ID NO: 56. It is envisaged that a ONC comprising or consisting of SEQ ID NO: 56 may be used to induce editing of the target adenosine 7247 in human Filamin A RNA (SEQ ID NO: 7). Accordingly, also the medical use of said ONC is envisaged herein. The sequence of SEQ ID NO: 56 in an ONC may have the modifications described below.
5′mA*mA*mA*GmA*rGmGrCrUrGrGrCrUrGmGrUrUmGrArCrCrUmUrUrArGmCrCrCmU rGrArCrUrCrCrCrGrArArGrGrCrUmArGmA*mA*mA*mC-3′
where
A is desoxyadenosine,
G is desoxyguanosine,
C is desoxycytosine,
T is desoxythymidine,
stands for phosphothioate linkage,
m stands for 2′ O-methyl ribose,
rU is uridine,
rA is adenosine,
rC is cytidine, and
rG is guanosine.

It is evident for the skilled person that the ONC described directly above may be extended in length on either end. The oligo may also be modified by addition of 2′ fluoro, 2′ O-methyl at different positions, pseudouridine, cholesterol or GalNac or any other modifications described herein or known in the art. Modifications may enhance stability, reduce immunogenicity and facilitate cellular uptake or promote endosomal escape.

To test whether ONCs are able to induce editing of target adenosines said ONCs may be transfected into mouse 3T3 cells using lipofectamine 3000, (Thermo Fisher, Waltham, MA) following the manufacturer's instructions and as described herein.

Preferably, the ONCs in context of the present invention bind to Filamin B RNA around the naturally edited adenosine and form dsRNA with Filamin B RNA that is recognized by endogenous ADAR. Said ONCs may bind to a sequence close to position 7123 of SEQ ID NO: 9. Preferably, the ONC binds to a sequence of SEQ ID NO: 9 including position 7123 of SEQ ID NO: 9. The ONC of the present invention may be perfectly complementary to the target sequence. However, the ONC may also contain several mismatches as long as the ONC binds to the target sequence under physiological conditions. Preferably, the ONC comprises a mismatch directly vis-à-vis the target adenosine. In other words, it is preferred that the ONC binds to a sequence of SEQ ID NO: 9 including position 7123 of SEQ ID NO: 9 and the nucleotide in the ONC facing the target adenosine does not comprise thymine or uracil. More preferably, the ONC binds to a sequence of SEQ ID NO: 9 including position 7123 of SEQ ID NO: 9 and is perfectly complementary to said sequence except for one mismatch directly vis-à-vis the target adenosine. Additional mismatches can be introduced to avoid off-target editing.

The sequences of the ONCs binding to Filamin B RNA may be selected from the group consisting of the sequences as depicted in SEQ ID NOs: 14 to 17 and sequences having at least 20%, preferably at least 25%, even more preferably at least 30%, even more preferably at least 40%, even more preferably at least 50%, even more preferably at least 60%, even more preferably at least 70%, even more preferably at least 80%, even more preferably at least 90%, even more preferably at least 95%, even more preferably at least 98% and most preferably at least 99% sequence identity to said sequences.

The skilled person is well aware how suitable sequences for ONCs, that bind to the desired target sequences can be designed. In principle, such ONCs may be at least 20 nucleotides in length to provide double-stranded structures of two helical turns. However, additional nucleotides may be added to provide better stability or allow the formation of specific shapes that are preferred by endogenous ADARs. So far, nucleotides of up to 150 nucleotides that are complementary to the editing region have been tested. Again, such extended oligos may form specific mismatches or carry specific modifications to reduce non-specific editing (for example Qu (2019) loc. cit.). The ONCs of the present invention may be also longer than 150 nucleotides as long as they are capable of modifying Filamin A or Filamin B. Likewise, it is also clear that the ONCs of the present invention may shorter than 20 nucleotides as long as they are capable of modifying Filamin A or Filamin B.

It is also known in the art that recruitment of endogenous ADAR can be optimized by mimicking optimal targets for ADAR (Merkle (2019) Nat Biotechnol 37:133-138; Katrekar (2019) Nat Methods 16:239-242). A sequence mimicking a optimal target for ADAR is termed herein “recruiting portion”.

Accordingly, the present invention also relates to pharmaceutical compositions comprising ONCs comprising
(a) a targeting portion, comprising a sequence complementary to part of the target RNA; and
(b) a recruiting portion capable of binding and recruiting an RNA editing entity naturally present in the cell or provided together with the oligonucleotide construct and capable of performing the editing of the nucleotide.

The target RNA is preferably Filamin A or Filamin B RNA. The RNA editing entity is preferably ADAR, preferably ADAR1 and/or ADAR2. The recruiting portion may comprise a sequence mimicking the Gria2 R/G site or any other well-defined substrate site such as the Gabra3 site (Ohlson (2007) Rna 13:698-703). Said sequence may be designed as described in the art (Merkle (2019) loc. cit.). The sequence mimicking the Gria2 R/G site may have the sequence as depicted in SEQ ID NO: 26 or a sequence having at least 20%, preferably at least 25%, even more preferably at least 30%, even more preferably at least 40%, even more preferably at least 50%, even more preferably at least 60%, even more preferably at least 70%, even more preferably at least 80%, even more preferably at least 90%, even more preferably at least 95%, even more preferably at least 98% and most preferably at least 99% sequence identity thereto.

When the target RNA is Filamin A RNA an ONC comprising a targeting portion and a recruiting portion may have the sequence selected from the group consisting of sequences as depicted in SEQ ID NOs: 18 to 21 and sequences having at least 20%, preferably at least 25%, even more preferably at least 30%, even more preferably at least 40%, even more preferably at least 50%, even more preferably at least 60%, even more preferably at least 70%, even more preferably at least 80%, even more preferably at least 90%, even more preferably at least 95%, even more preferably at least 98% and most preferably at least 99% sequence identity to said sequences.

When the target RNA is Filamin B RNA an ONC comprising a targeting portion and a recruiting portion may have a sequence selected from the group consisting of sequences as depicted in SEQ ID NOs: 22 to 25 and sequences having at least 20%, preferably at least 25%, even more preferably at least 30%, even more preferably at least 40%, even more preferably at least 50%, even more preferably at least 60%, even more preferably at least 70%, even more preferably at least 80%, even more preferably at least 90%, even more preferably at least 95%, even more preferably at least 98% and most preferably at least 99% sequence identity to said sequences.

The pharmaceutical compositions as described herein may further comprise a substance stimulating the expression of endogenous ADAR. Preferably, the substance stimulating the expression of endogenous ADAR is interferon alpha or interferon beta.

The pharmaceutical compositions as described herein may also further comprise a nucleic acid molecule encoding ADAR1 and/or ADAR2, or a vector comprising a nucleic acid molecule encoding ADAR1 and/or ADAR2.

The present invention relates to an ONC as described herein for use as a medicament. Accordingly, the present invention relates to an ONC as described herein for use in the treatment of a disease. Likewise, the present invention relates to an ONC as described herein for use in the preparation of a medicament for the treatment of a disease. Said disease may be a proliferative or inflammatory disease. Said disease may also be a skin lesion, mucosa lesion or another lesion of a structure of the body.

It is also envisaged herein that the RNA editing entity capable of performing the editing of the nucleotide is not naturally present in the cell. Accordingly, the present invention also relates to a pharmaceutical composition comprising an ONC that may recruit to the target RNA an RNA editing entity not naturally present in the cell and capable of performing the editing of the nucleotide.

Furthermore, it is envisaged that rationally designed ADAR-fusions comprising the ADAR catalytic domain are used for site directed RNA-editing. Accordingly, the present invention also relates to a pharmaceutical composition comprising an ONC that may recruit to the target RNA an RNA editing entity not naturally present in the cell and capable of performing the editing of the nucleotide, wherein the RNA editing entity not naturally present in the cell comprises an ADAR catalytic domain.

The ADAR catalytic domain may be an ADAR1 catalytic domain. The ADAR1 catalytic domain may have a sequence as depicted in SEQ ID NO: 34 or a sequence having at least 20%, preferably at least 25%, even more preferably at least 30%, even more preferably at least 30%, even more preferably at least 40%, even more preferably at least 50%, even more preferably at least 60%, even more preferably at least 70%, even more preferably at least 80%, even more preferably at least 90%, even more preferably at least 95%, even more preferably at least 98% and most preferably at least 99% sequence identity thereto.

The ADAR catalytic domain may be an ADAR2 catalytic domain. The ADAR2 catalytic domain may have a sequence as depicted in SEQ ID NO: 35 or a sequence having at least 20%, preferably at least 25%, even more preferably at least 30%, even more preferably at least 30%, even more preferably at least 40%, even more preferably at least 50%, even more preferably at least 60%, even more preferably at least 70%, even more preferably at least 80%, even more preferably at least 90%, even more preferably at least 95%, even more preferably at least 98% and most preferably at least 99% sequence identity thereto.

The skilled person is well aware that hyperactive variants of ADAR2 catalytic domains have been identified (Kuttan (2012) Proc Natl Acad Sci USA 109:E3295-3304). Accordingly, the skilled person understands that hyperactive ADAR catalytic domains may be used in context of the present invention.

The RNA editing entities not naturally present in the cell may comprise a recruiting domain. The term “recruiting domain” as used herein refers to a polypeptide that is able to bind to a certain target RNA. The recruiting domain may be fused to additional proteins or protein domains and recruit said additional proteins or protein domains to the target RNA. It is envisaged that the recruiting domain is part of an RNA editing entity and recruits said RNA editing entity to the target RNA. For example, there are RNA binding proteins/domains known in the art that associate with oligonucleotides and then specifically bind to another nucleotide sequence complementary to the associated oligonucleotide. Accordingly, it is envisaged that an ONC in context of the present invention associates with a recruiting domain, preferably an RNA binding protein/domain and triggers the binding of the recruiting domain to a target RNA complementary to said ONC. Therefore, it is envisaged herein that said RNA binding protein/domains are fused to an ADAR catalytic domain and recruit the ADAR catalytic domain to the target RNA. Accordingly, the present invention also relates to a pharmaceutical composition comprising an ONC that recruits to the target RNA an RNA editing entity not naturally present in the cell and capable of performing the editing of the nucleotide, wherein the RNA editing entity not naturally present in the cell comprises an ADAR catalytic domain and an recruiting domain, wherein the recruiting domain associates with said ONC and binds to the target RNA upon association with said ONC.

The skilled person knows which proteins can be used as recruiting domains. Proteins or protein domains that can be used as recruiting domains include but are not limited to SNAP-tag (Vogel (2018) Nat Methods 15:535-538), lambda N protein (Montiel-Gonzalez (2013) Proc Natl Acad Sci USA 110:18285-18290), MS2 coat protein (Betrand (1998) Mol Cell 2:437-445), CAS13 (Cox (2017) Science 358:1019-1027) and CIRTS (Rauch (2019) Cell 178:122-134 e112). The present invention, thus, also relates to a pharmaceutical composition comprising an ONC that recruits to the target RNA an RNA editing entity not naturally present in the cell and capable of performing the editing of the nucleotide, wherein the RNA editing entity comprises an ADAR catalytic domain and an recruiting domain, wherein the recruiting domain associates with said ONC and binds to the target RNA upon association with said ONC and wherein the recruiting domain is selected from the group consisting of SNAP-tag, lambda N protein, MS2 coat protein, CAS13 and CIRTS.

When the recruiting domain in an RNA editing entity comprising an ADAR catalytic domain and a recruiting domain is the lambda N protein the amino acid sequence of the RNA editing entity may be SEQ ID NO: 30 or SEQ ID NO: 31 or a sequence having at least 20%, preferably at least 25%, even more preferably at least 30%, even more preferably at least 40%, even more preferably at least 50%, even more preferably at least 60%, even more preferably at least 70%, even more preferably at least 80%, even more preferably at least 90%, even more preferably at least 95%, even more preferably at least 98% and most preferably at least 99% sequence identity to said sequences.

When the recruiting domain in an RNA editing entity comprising an ADAR catalytic domain and a recruiting domain is the SNAP tag the amino acid sequence of the RNA editing entity may be SEQ ID NO: 32 or SEQ ID NO: 33 or a sequence having at least 20%, preferably at least 25%, even more preferably at least 30%, even more preferably at least 40%, even more preferably at least 50%, even more preferably at least 60%, even more preferably at least 70%, even more preferably at least 80%, even more preferably at least 90%, even more preferably at least 95%, even more preferably at least 98% and most preferably at least 99% sequence identity thereto. When the recruiting domain in an RNA editing entity comprising an ADAR catalytic domain and a recruiting domain is the MS2 coat protein the amino acid sequence of the RNA editing entity may be SEQ ID NO: 37 or SEQ ID NO: 39 or a sequence having at least 20%, preferably at least 25%, even more preferably at least 30%, even more preferably at least 40%, even more preferably at least 50%, even more preferably at least 60%, even more preferably at least 70%, even more preferably at least 80%, even more preferably at least 90%, even more preferably at least 95%, even more preferably at least 98% and most preferably at least 99% sequence identity thereto. When the recruiting domain in an RNA editing entity comprising an ADAR catalytic domain and a recruiting domain is the MS2 coat protein said RNA editing entity may further comprise a nuclear localization signal. The amino acid sequence of an RNA editing entity comprising an ADAR catalytic domain, the MS2 coat protein and a nuclear localization signal may be SEQ ID NO: 36 or SEQ ID NO: 38 or a sequence having at least 20%, preferably at least 25%, even more preferably at least 30%, even more preferably at least 40%, even more preferably at least 50%, even more preferably at least 60%, even more preferably at least 70%, even more preferably at least 80%, even more preferably at least 90%, even more preferably at least 95%, even more preferably at least 98% and most preferably at least 99% sequence identity thereto.

Although clear for the skilled person it is noted that all other RNA editing entities described herein or useful in context of the present invention may be fused to a nuclear localization signal.

It is clear that the present invention also relates to ONCs that may recruit to the target RNA an RNA editing entity not naturally present in the cell and capable of performing the editing of the nucleotide for use as a medicament. Accordingly, the present invention relates to said ONC for use in the treatment of a disease. Likewise, the present invention relates to said ONC as described herein for use in the preparation of a medicament for the treatment of a disease. Said disease may be a proliferative or inflammatory disease. Said disease may also be a skin lesion, mucosa lesion or another lesion of a structure of the body.

The present invention also relates to the RNA editing entity not naturally present in the cell for use as a medicament. Accordingly, the present invention relates to said RNA editing entity for use in the treatment of a disease. Likewise, the present invention relates to said RNA editing entity for use in the preparation of a medicament for the treatment of a disease. Said disease may be a proliferative or inflammatory disease. Said disease may also be a skin lesion, mucosa lesion or another lesion of a structure of the body.

The present invention also provides a nucleic acid encoding the RNA editing entity not naturally present in the cell as described herein for use as a medicament. Likewise, the invention provides vectors comprising a nucleic acid encoding an RNA editing entity not naturally present in the cell as described herein for use as a medicament. Accordingly, the invention also relates to the combination of a nucleic acid or a vector encoding an RNA editing entity not naturally present in the cell and an ONC for use as a medicament, wherein the ONC recruits the RNA editing entity to the target RNA. The present invention also relates to said combination for use in the treatment of a disease. Likewise, the present invention relates to said combination for use in the preparation of a medicament for the treatment of a disease. Said disease may be a proliferative or inflammatory disease. Said disease may also be a skin lesion, mucosa lesion or another lesion of a structure of the body.

The ONCs as defined herein may comprise or consist of DNA and/or RNA. Preferably, the ONCs according to the present invention comprise or consist of RNA.

Although clear for the skilled person it is noted that ONCs according to the present invention may comprise one or more residues that are modified to increase nuclease resistance, and/or to increase the affinity of the antisense oligonucleotide for the target sequence. The ONCs according to the present invention may comprise at least one nucleotide analogue, wherein a nucleotide analogue is defined as a residue having a modified base, and/or a modified backbone, and/or a non-natural internucleoside linkage, or a combination of these modifications.

Thus, ONCs as defined herein may comprise at least one nucleotide analogue, which comprises a modified backbone. Examples of such backbones include but are not limited to morpholino (phosphorodiamidate) backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones.

The linkage between the nucleotide residues in a backbone may not include a phosphorus atom, such as a linkage that is formed by short chain alkyl or cycloalkyl internucleoside linkages or mixed heteroatom alkyl or cycloalkyl internucleoside linkages. A nucleotide analogue may comprise a Peptide Nucleic Acid (PNA), having a modified polyamide backbone (Nielsen (1991) Science 254:1497-1500). Since the backbone of a PNA molecule contains no charged phosphate groups, PNA-RNA hybrids are usually more stable than RNA-RNA or RNA-DNA hybrids, respectively (Egholm (1993) Nature 365:566-568). In another nucleotide analogue the ribose or deoxyribose sugar may be replaced by a 6-membered morpholino ring. In another nucleotide analog the ribose or deoxyribose sugar is replaced by a 6-membered morpholino ring, and the anionic phosphodiester linkage may be replaced by a non-ionic phosphorodiamidate linkage.

The nucleotide analogue may comprise a substitution of one of the non-bridging oxygens in the phosphodiester linkage. This modification slightly destabilizes base-pairing but adds significant resistance to nuclease degradation. Accordingly, the nucleotide analogue may comprise phosphorothioate, phosphorodithioate, phosphotriester, aminoalkylphosphotriester, H-phosphonate, methyl and other alkyl phosphonate including 3′-alkylene phosphonate, 5′-alkylene phosphonate, phosphinate, phosphoramidate including 3′-amino phosphoramidate and amino alkylphosphoramidate, thionophosphoramidate, thionoalkylphosphonate, thionoalkylphosphotriester, selenophosphate or boranophosphate. Preferably, the nucleotide analogue comprises phosphorothioate.

The sugar moiety can be a pyranose or derivative thereof, or a deoxypyranose or derivative thereof, preferably ribose or derivative thereof, or deoxyribose or derivative thereof. A further nucleotide analogue may comprise a modified sugar. The modified sugar may be substituted at the 2′, 3′ and/or 5′ position. Said positions may be substituted by —OH, —F, linear or branched lower (C1-C10) alkyl, alkenyl, alkynyl, alkaryl, allyl, and/or aralkyl groups, that may be interrupted by one or more heteroatoms; Said positions may be also substituted by 0-, S—, or N-alkyl; 0-, S—, or N-alkenyl; 0-, S-or N-alkynyl; 0-, S—, or N— allyl; and/or O-alkyl-O-alkyl groups. In addition, said positions may be substituted by methoxy-, aminopropoxy-, methoxyethoxy-, dimethylaminooxyethoxy-, and/or dimethylaminoethoxyethoxy-groups. A derivatized sugar moiety may also comprise a Locked Nucleic Acid (LNA) or an Unlocked Nucleic Acid (UNA), preferably an LNA. In a preferred embodiment the sugar modification is selected from the group consisting of Locked Nucleic Acid (LNA), Unlocked Nucleic Acid (UNA), 2′-fluororibose and 2′-O-alkyl modifications. Preferably, the 2′-O-alkyl modification may be a 2′-O-methyl modification.

Furthermore, it is envisaged that chemical modifications to stabilize RNAs and to reduce immunogenicity are introduced to RNA oligonucleotides. These include, but are not restricted/limited to: phosphothioate linkers, 2′O methylation, 2′ fluoro, locked nucleic acids, peptide nucleic acids, pseudouridine, or 5-methyl-cytidine (for further details see Roberts (2020) Nat Rev Drug Discov 19:673-694)

It is clear for the skilled person that it is not necessary for all positions in an ONC to be modified uniformly. In addition, more than one of the aforementioned analogues may be incorporated in a single ONC. In certain embodiments, an ONC according to the present invention has at least two different types of analogues or equivalents.

Modifications of ONCs can also facilitate delivery to specific compartments or cell types. For instance, cholesterol linkers allow the interaction with lipoprotein particles and subsequent phagocytosis in the liver (Wolfrum (2007) Nat Biotechnol 25:1149-1157).

Similarly, linking of ONCs to antibodies can be used to target specific cell types (Tushir-Singh (2016) Expert Opin. Biol. Ther. 17,3: 325-338).

Linking ONCs to membrane penetrating peptides may help to liberate the oligos from the endocytic vesicles. Peptide linkage may also be used to deliver oligos to specific cell types when the peptide links to specific receptors (Aemmaelae (2018) Sci. Ad. 4:eaat3386). Furthermore, chemical modifications like GalNac linkage can be used to boost hepatocyte uptake of ONCs (Brown (2020) Nucleic Acid Res. 48,21:11827-11844).

It is also envisaged that delivery of ONCs to endothelial cells and uptake of ONCs by endothelial cells may be facilitated/enhanced via specific modifications. Delivery of ONCs to endothelial cells and uptake of ONCs by endothelial cells may be relevant when ONCs are used to prevent angiogenesis, e.g. in cancer treatment.

To boost uptake of oligos into endothelial cells to prevent angiogenesis, modified oligos may be coupled to CD31 antibodies (Chacko (2015) ACS Nano 9,7:6785-6793). Furthermore, linkage to membrane penetrant peptides or integrin binding peptides may be used.

Also delivery via lipid nanoparticles may be used to target endothelial cells (Kowalski (2011) IUBMB Life 63(8):648-658; Khan (2018) Sci. Ad. 4,6:eaar8409).

In addition, it is envisaged that ONCs described herein are delivered to intestinal epithelial cells. Delivery of ONCs to intestinal endothelial cells and uptake of ONCs by endothelial cells may be relevant inter alia when ONCs are used to treat e.g. inflammatory diseases of the intestine such as colitis. For delivery to intestinal endothelial cells rectal and oral delivery is envisaged. As a non-limiting example lipid and/or lipidoid nanoparticles may be used for oral and rectal delivery. As a non-limiting example the oligonucleotides may be cholesterol coupled (Ball (2015) Plos One 10(7):e0133154).

The invention also relates to the ONCs described herein.

The pharmaceutical compositions as described herein may be in solid/lyophilized or liquid form and may be, e.g., in a form of (a) powder(s), (a) tablet(s), (a) solution(s) or (an) aerosol(s). Furthermore, it is envisaged that the pharmaceutical compositions as described herein may comprise further biologically active agents, depending on the intended use of the pharmaceutical composition. The pharmaceutical compositions as described herein may further comprise a pharmaceutically acceptable carrier, excipient and/or diluent. Examples of suitable pharmaceutical carriers, excipients and/or diluents are well known in the art and include but are not limited to phosphate buffered saline solutions or other buffer solutions, water, emulsions, such as oil/water emulsions and various types of wetting agents. Compositions comprising such carriers can be formulated by well known conventional methods. Suitable carriers may comprise any material as long as the polypeptide, nucleic acid, vector or ONC described herein retains its biological and/or pharmaceutical activity upon contact with said carriers. Pharmaceutical compositions for parenteral administration may include sterile aqueous or non-aqueous solutions, suspensions, and emulsions. Examples of non-aqueous solutions include but are not limited to propylene glycol, polyethylene glycol, vegetable oils such as olive oil, and injectable organic esters such as ethyl oleate. Aqueous solutions include water, alcoholic/aqueous solutions, including saline and buffered media. Pharmaceutical compositions for parenteral administration may further include sodium chloride solution, Ringer's dextrose, dextrose and sodium chloride or lactated Ringer's. Pharmaceutical compositions for intravenous administration may include fluid and nutrient replenishers, electrolyte replenishers (such as those based on Ringer's dextrose), and the like. The pharmaceutical compositions as described herein may also comprise preservatives and other additives including but not limited to antimicrobials, anti-oxidants, chelating agents and/or inert gases and the like. In addition, a pharmaceutical composition as described herein may comprise proteinaceous carriers, like, e.g., serum albumin or immunoglobulin, preferably of human origin.

Administration of the pharmaceutical compositions as described herein may be effected by different ways, e.g., by parenteral, subcutaneous, intravenous, intraarterial, intraperitoneal, topical, intrabronchial, intrapulmonary and intranasal administration and, if desired for local treatment, intralesional administration. The pharmaceutical compositions as described herein may also be administered directly to the target site, e.g., by biolistic delivery to an external or internal target site, like a specifically effected organ. In particular, delivery of an oligonucleotide to induce an adenosine to inosine exchange in vascular endothelial cells with the aim to reduce vascular outgrowth in, e.g., tumors would occur for example via intravenous injection of lipid nanoparticles loaded with ONCs as described herein. Similarly, proteins, nucleic acid molecules or vectors as described herein may be delivered. To target inflammatory processes in the large intestine proteins, nucleic acid molecules, vectors or ONCs as described herein could be packaged in lipid nanoparticles or AAV vectors. Intestinal epithelial cells could be reached via an enema of a solution containing proteins, nucleic acid molecules, vectors or ONCs as described herein. Intestinal epithelial cells could also be reached by oral delivery of suitably packaged lipid nanoparticles or AAV vectors delivering the ONC of interest via a slow release such as dissolution or osmotic systems.

In order to reach wounded skin the proteins, nucleic acid molecules, vectors or ONCs as described herein would be applied ectopically in lipid nanoparticles or AAV vectors in an emulsion.

The invention further relates to a method of treatment of a disease comprising administering the pharmaceutical composition as described herein to a subject in need thereof. The invention further relates to a method of treatment of a disease comprising administering a modified Filamin A and or modified Filamin B as described herein to a subject in need thereof. In addition, the invention relates to a method of treatment of a disease comprising administering a nucleic acid molecule encoding a modified Filamin A and/or modified Filamin B as described herein to a subject in need thereof. Furthermore, the invention relates to a method of treatment of a disease comprising administering a vector comprising a nucleic acid molecule encoding a a modified Filamin A and/or modified Filamin B to a subject in need thereof. The invention also relates to a method of treatment of a disease comprising administering an ONC as described herein to a subject in need thereof. The invention further relates to a method of treatment of a disease comprising administering to a subject in need thereof an ONC as described herein and an RNA editing entity, preferably ADAR1 or ADAR2 recruited to the target RNA by said ONC. The invention further relates to a method of treatment of a disease comprising administering to a subject in need thereof an ONC as described herein and a nucleic acid molecule or a vector encoding an RNA editing entity, preferably ADAR1 or ADAR2 recruited to the target RNA by said ONC. The invention further relates to a method of treatment of a disease comprising administering to a subject in need thereof an ONC as described herein and an RNA editing entity not naturally present in the cell recruited to the target RNA by said ONC and as described herein above. The invention further relates to a method of treatment of a disease comprising administering to a subject in need thereof an ONC as described herein and a nucleic acid or vector encoding an RNA editing entity not naturally present in the cell recruited to the target RNA by said ONC and as described herein above.

The term “disease” is used herein in the broadest sense. Preferably the disease is a proliferative disease or an inflammatory disease. The term proliferative disease is used herein in the broadest sense. A non-limiting example for a proliferative disease is cancer. The term “cancer” is used herein in the broadest sense and refers, e.g., to carcinoma, sarcoma, melanoma, lymphoma and leukemia. Examples for cancer include but are not limited to bladder cancer, brain cancer, breast cancer, ovarial cancer, cervical cancer, testicular cancer, prostate cancer, colon cancer, gastric cancer, esophageal cancer, liver cancer, lung cancer and pancreatic cancer. A preferred disease treated by means and methods of the present invention is melanoma. Examples for melanoma include but are not limited to cutaneous melanoma, uveal melanoma, mucosal melanoma, and metastatic melanoma.

The term “inflammatory disease” is used herein in the broadest sense and may include but is not limited to IBD. Examples for IBD include but are not limited to Crohn's disease and ulcerative colitis. A preferred disease treated by means and methods of the present invention is colitis.

Disease as used herein also refers to lesions of the skin, to lesions of mucosa and to other lesions of structures of the body.

Accordingly, the invention relates to the pharmaceutical compositions described herein for use in the treatment of a proliferative disease or an inflammatory disease. The invention further relates to pharmaceutical compositions described herein for use in the treatment of cancer. The invention also relates to pharmaceutical compositions described herein for use in the treatment of bladder cancer, brain cancer, breast cancer, cervical cancer, colon cancer, gastric cancer, liver cancer, lung cancer and pancreatic cancer. Particularly, the invention relates to pharmaceutical compositions described herein for use in the treatment of melanoma, such as cutaneous melanoma, uveal melanoma, mucosal melanoma, and metastatic melanoma. In addition, the invention also relates to pharmaceutical compositions described herein for use in the treatment of IBD, preferably Crohn's disease and colitis. More preferably the invention relates to pharmaceutical compositions described herein for use in the treatment of colitis.

The invention also relates to pharmaceutical compositions described herein for use in the treatment of a skin lesion, mucosa lesion or another lesion of a structure of the body.

Accordingly, the invention relates to a method of treatment of a proliferative disease or an inflammatory disease. The invention further relates to a method of treatment of cancer. The invention also relates a method of treatment of bladder cancer, brain cancer, breast cancer, cervical cancer, colon cancer, gastric cancer, liver cancer, lung cancer and pancreatic cancer. Particularly, the invention relates a method of treatment of melanoma, such as cutaneous melanoma, uveal melanoma, mucosal melanoma, and metastatic melanoma. In addition, the invention also relates to a method of treatment of IBD, preferably Crohn's disease and colitis. More preferably the invention relates to a method of treatment of colitis.

The invention also relates to a method of treatment of a skin lesion, mucosa lesion or another lesion of a structure of the body.

The skilled person is aware that administration methods/routes may vary depending on the disease to be treated. The skilled person is readily in the position to choose suitable administration methods/routes depending on the disease to be treated.

The terms “modified” and “edited” is used interchangeably herein when referring to Filamin A or Filamin B protein and Filamin A or Filamin B RNA. Filamin A and FLNA is used synonymously herein. FLNA^{R} as used herein refers to Filamin A polypeptide with an arginine corresponding to the arginine at position 2333 of SEQ ID NO: 1. FLNA^{Q} as used herein refers to Filamin A polypeptide with a glutamine corresponding to the glutamine at position 2333 of SEQ ID NO: 27. FLNA^{R} mice as used herein refers to mice exclusively producing Filamin A protein with an arginine corresponding to the arginine at position 2333 of SEQ ID NO: 1. FLNA^{Q} mice as used herein refers to mice exclusively producing Filamin A with a glutamine corresponding to the glutamine at position 2333 of SEQ ID NO: 27.

The present invention is illustrated by the following Figures and Examples.

### FIGURES

FIG. 1: Generation of a mouse expressing constitutively edited FLNA^{R}

(A) Two cuts were introduced in fertilized oocytes in the FLNA gene using CRISPR/Cas technology. The targeted sequences are gRNA1 (matching forward strand of gene):

(SEQ ID NO: 40)
CTGTTTCTAGTCTTCAGGTGAGG

and g matching reverse strand of gene):

(SEQ ID NO: 41)
GCTACCTGGGTGTGTGATTGTGG

(PAM sequences are underlined). The oocytes were coinjected with a repair template (Donor template; SEQ ID NO: 49). The targeted allele was verified by sequencing.

(B) The repair template replaces an adenosine by a guanosine at position 74.226.862 on chromosome X (mm10). The relevant part of said chromosome is shown (SEQ ID NO:54). Note, FLNA is encoded on the reverse strand, leading to a A to G exchange in the transcribed RNA. The nucleotide exchange is bold-face and underlined. Exon sequences are capitalized, intronic sequences are lower case. Sequence in sense (transcript) orientation.

FIG. 2: Genotyping of FLNA wild type, FLNA^{Q} and FLNA^{R} mice.

Tail-clip DNA was extracted and tested by PCR for the internal deletion of the editing complementary site in intron 43. Wild-type animals give the largest band, FLNA^{Q} animals the second largest band due to deletion of the editing complementary sequence (ECS). FLNA^{R} animals that yield the smallest band since the ECS is deleted but the CRISPR/Cas9 generated mice also lack remnants of LoxP sequences. Genotyping primer forward:

(SEQ ID NO: 42)
TCTGGATGGTAGGCTTCTGC;

### Genotyping Primer Reverse

(SEQ ID NO: 43)
CTGGAAGCATAGCAGATGTGG.

FIG. 3: Comparison of tumor growth in FLNA^{Q} and FLNA^{R} mice

(A) Tumor images derived from subcutaneously injected B16F10 (skin melanoma) cells injected into FLNA^{Q} (unedited) and FLNA^{R} (edited) mice. (B) Graph showing tumor weight (in g) compared between FLNA^{Q} (unedited) and FLNA^{R} (edited) mice. (C) Graph showing tumor volume (mm³) compared between FLNA^{Q} (unedited) and FLNA^{R} (edited) mice. FLNA^{Q} (unedited mice) clearly show bigger tumors with both tumor weights (5-fold) and volumes (6-fold) statistically significant (P<0.01).

FIG. 4: Comparison of tumor vascularization in FLNA^{Q} and FLNA^{R} mice

(A) Tumor sections stained with α-CD31 to detect blood vessels in xenograft tumors in both FLNA^{Q} and FLNA^{R} mice. Inset shows a magnified area in the respective tumor represented by a white box on the whole tumor section. Note that the scale bars of the images are different. (B, C) Graphs showing vascular density (number of vessels/mm²) compared between FLNA^{Q} (unedited) and FLNA^{R} (edited) xenograft tumors measured across the biggest section (B) and after averaging 3 sections from the same tumor (C). Both measurements are highly significant with FLNA^{Q} tumors having at least 2-fold more vessel density. P values are indicated on the graphs. At least 4 tumors were analyzed per genotype.

FIG. 5: Model showing the effect of FLNA RNA editing on tumor growth and neo-angiogenesis

Once FLNA RNA is edited by ADAR enzymes, it leads to a glutamine to arginine conversion in the repeat 22 of FLNA protein. Injection of melanoma cells into edited mouse gives rise to a much smaller tumor as a consequence of reduced vascular supply (right side). On the other hand, when ADARs fail to edit FLNA mRNA, it leads to a robust vascular supply in the growing tumor fostering a much bigger sized tumor (left side).

FIG. 6: Transwell migration assay of endothelial cells

Endothelial cells were grown in a transwell chamber and allowed to migrate towards media containing VEGF. (A) Cells only expressing unedited FLNA^{Q} migrate faster than cells expressing edited FLNA^{R} (B). (C) Quantification of transwell-migration assay shown in A and B. The graph shows ˜2 fold more cell migration in unedited FLNA^{Q} cells towards VEGF as a chemoattractant in the lower chamber. The experiment was repeated in triplicates. Scale bar: 200 μm. P<0.05.

FIG. 7: 3D spheroid sprouting assay

3D Spheroids were generated by embedding endothelial cells only expressing unedited FLNA^{Q} (A) or edited FLNA^{R} (B). Sprouting was analyzed after 72 hrs. FLNA^{Q} expressing cells show increased sprouting of cells. The experiment was done in triplicates. Scale bar: 50 μm.

FIG. 8: Aortic ring sprouting assay

Brightfield images showing representative aortic sprouts at day 4 made from aortas of mice expressing only unedited FLNA^{Q} (A) and from aortas of mice expressing only edited FLNA^{Q} (B). The graph (C) shows ˜2 times longer sprouts in unedited (FLNA^{Q}) aortas as compared to edited (FLNA^{R}) counterparts. 4 independent mice were used for the assay and at least 3 rings/mice were used. Scale bar: 200 μm. P<0.05

FIG. 9: H&E stained colon sections of DSS treated wild type and FLNA^{Q} mice

Representative H&E stained colon sections showing colon histology from rectum (inside) to caecum (outside) after DSS treatment in wild type mouse (A, C) and FLNA^{Q} mouse (B, D). C and D are the magnified images of A and B respectively. Arrows mark the unaffected epithelium and arrowheads indicate the eroded epithelial lining that is more prominently seen in FLNA^{Q} samples after DSS treatment. Six mice were used for the analysis from each genotype.

FIG. 10: Quantification of inflammation status in DSS treated mice Colon sections were scored blinded according to their inflammation status. The scoring was graded from 0-10. (A) The total score was determined in the whole colon and compared across wt, FLNA^{Q} and FLNA^{R} mice. Similarly, the scoring was determined across (B) the distal part (near rectum); (C) middle part and (D) proximal part (near caecum) of the colon. Six mice were used for the analysis from each genotype. One-way ANOVA test was done to measure the significance. P<0.05 was considered to be significant.

FIG. 11: Quantification of inflammation markers in DSS treated mice

Colon sections were graded for characteristic features of DSS induced colitis including cellular infiltration (A), Crypt damage (B), epithelial cell erosion (C) and thickening (D) and compared across wt, FLNA^{Q} and FLNA^{R} genotypes. Six mice were used for the analysis from each genotype. One-way ANOVA test was done to measure the significance. P<0.05 was considered to be significant (denoted by an asterisk *).

FIG. 12: Comparison of colon inflammation in FLNA^{Q} and FLNA^{R} mice Graphs showing the colitis scoring compared directly between FLNA^{Q} and FLNA^{R} mice. (A-D) Colitis inflammation induced after DSS treatment graded in the whole colon (A), distal part (B), middle part (C) and the proximal part (D). (E-H) DSS induced colon sections were scored for cellular infiltration (E), crypt damage (F), epithelial cell erosion (C) and thickening (D) and compared between FLNA^{Q} and FLNA^{R} mice. Data significance was tested by t-test. * P<0.05, ** P<0.01, ns=non-significant.

FIG. 13: Loss of FLNA editing leads to reduced wound healing.

Four wild-type and four FLNA^{Q} mice were wounded using a 4 mm diameter biopsy puncher by punching through the dorsal shaved skin after forming a pouch so that two symmetric wounds are generated at both sides of the pouch. Following the following 10 days wound healing is monitored. FLNA^{Q} mice show a clearly reduced wound closure which is most prominent 4-6 days after introducing the wound p<0.05.

FIG. 14: Site-directed RNA editing induced by delivery of modified oligonucleotides antisense to filamin mRNA in mouse 3T3 cells

(A) Raw sequence chromatogram of parts of endogenous filamin cDNA (SEQ ID NO: 55) after delivery of the ONC. (B) Chromatogram of parts of endogenous filamin cDNA after delivery of the ONC. (C) Chromatogram of parts of endogenous filamin cDNA without delivery of the ONC.

(A) and (B): Arrow marks position 7245 in mouse Filamin A mRNA (XM_036161857.1; SEQ ID NO: 50). The conversion of A to G leads to a shoulder in the G-trace underneath the prominent A peak. The A to G conversion leads to a Q to R exchange in the encoded protein. An Asterisk marks position 7258 in mouse Filamin A mRNA where a G peak can be seen underneath the A peak in. This second A to G exchange leads to a synonymous codon exchange. C) In the absence of the ONC no conversion of A to G can be observed at either position 7245 or position 7258.

### EXAMPLE 1: GENERATION OF MICE HOMOZYGOUS FOR FLNA VARIANTS CONSTITUTIVELY EDITED OR CONSTITUTIVELY UNEDITED

In order to test whether FLNA mRNA editing affects pathological processes, studies in transgenic mice were conducted. To generate mice unable to edit the FLNA pre mRNA, the intronic editing complementary sequence was deleted using homologous recombination. Generation of the mouse and the technology was described previously (Jain (2018) loc. cit.). Briefly, a construct for homologous recombination (knockout construct) was cloned. The construct spanned intron 28 to intron 45 and was inserted into a vector containing the diphteria toxin. The editing complementary sequence spanning nucleotide 23129 to 23330 in Gene assembly NC_000086.7 was replaced by a selectable neomycin cassette flanked by two lox P sites. The linearized, homologous recombination construct (SEQ ID NO: 48) was introduced into mouse ES cells by electroporation. After 10 days of selection under G418 positive colonies were picked and tested for insertion of the knockout cassette by southern blotting. Positive clones were further cultivated and the neomycin cassette was removed by transfection of Cre-recombinase. Cells were tested for positive expression and splicing of FLNA. The ES cells were used for blastocyst injection into B16 blastocysts and chimeric mice were selected by fur pigmentation. After crossing chimeric mice to wild type B16 mice positive founder individuals were identified by PCR of tailclip DNA. The deletion and lack of FLNA editing was verified by Sanger sequencing. Positive individuals were backcrossed 5 times to C57B16 mice.

Generation of a constitutively edited FLNA^{R} mouse. To generate mice constitutively edited in the FLNA gene, CRISPR-Cas9 technology was used:

To do so, the editing complementary site (ECS) required for generating the double stranded RNA and forming the ADAR target site was deleted and the editing site itself was also permanently edited (from CAG to CGG, position 74226862 on chromosome X of mm10). Briefly, guide RNAs surrounding the ECS deletion region were used to make a cut by Cas-9. Forward target sequence

(SEQ ID NO: 40)
CTGTTTCTAGTCTTCAGGTGAGG,

reverse target sequence

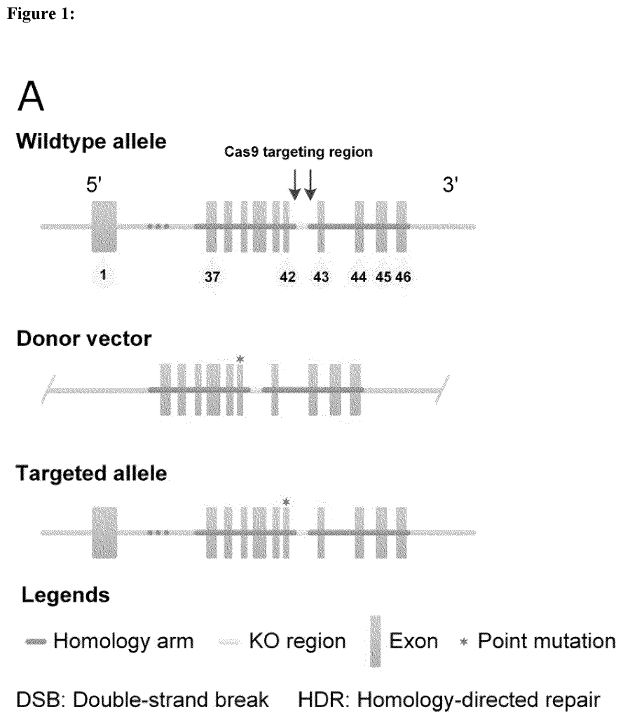

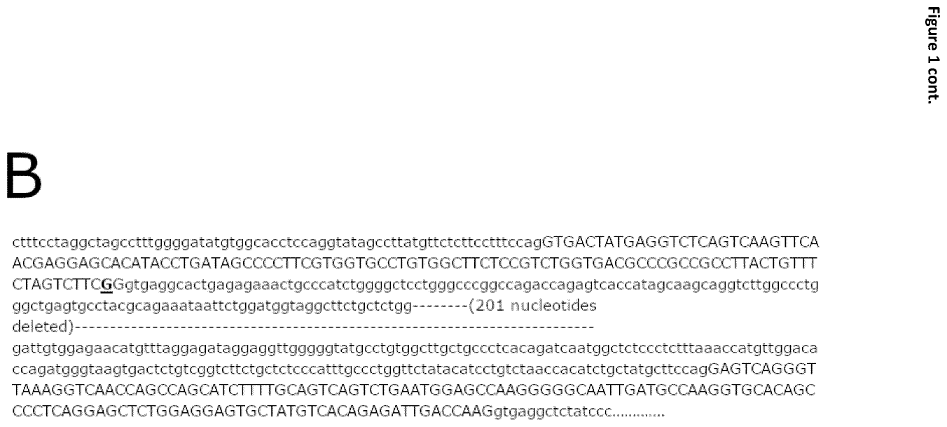

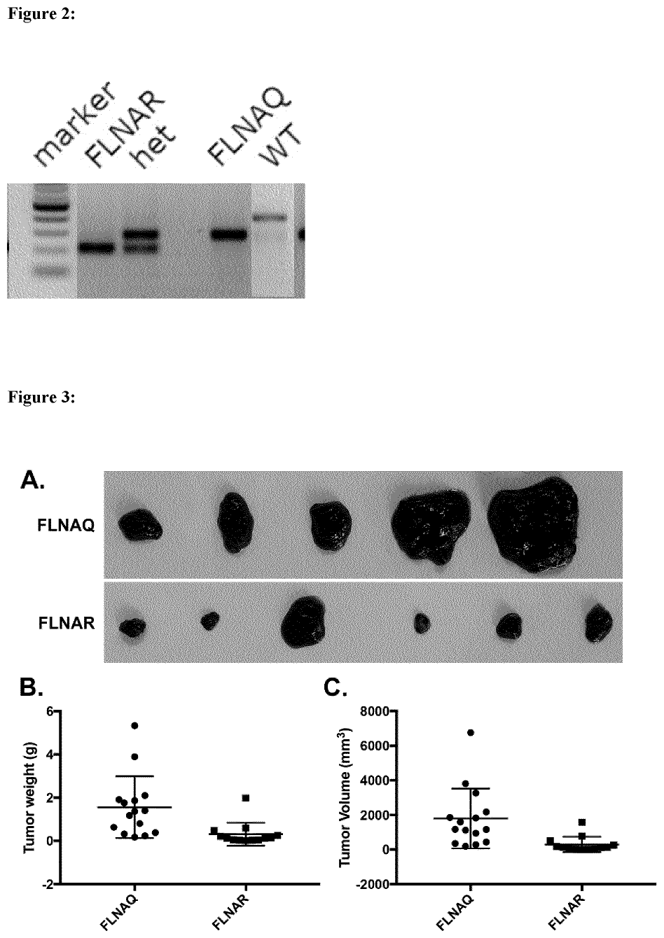

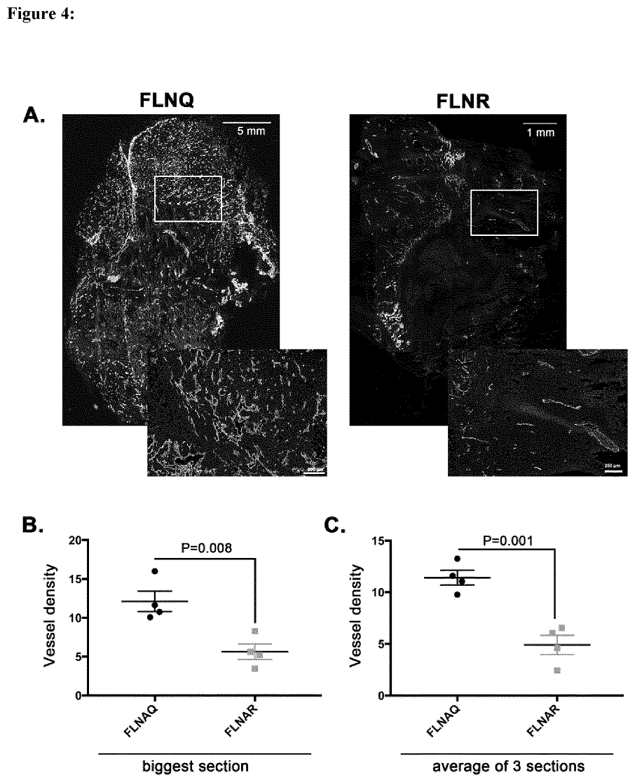

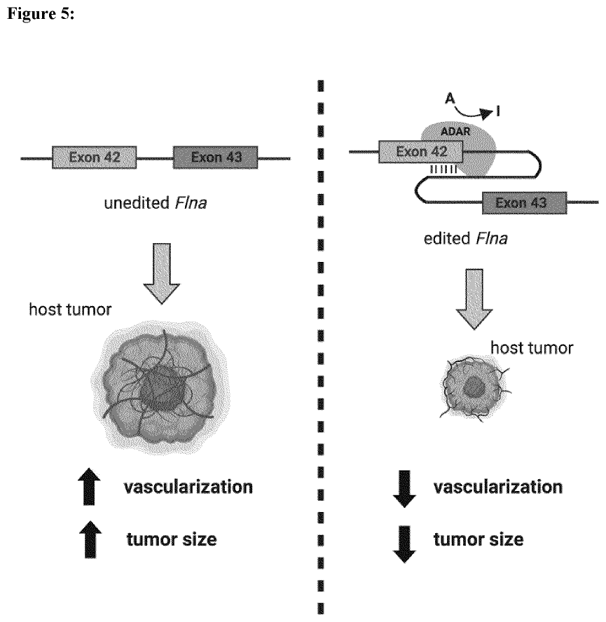

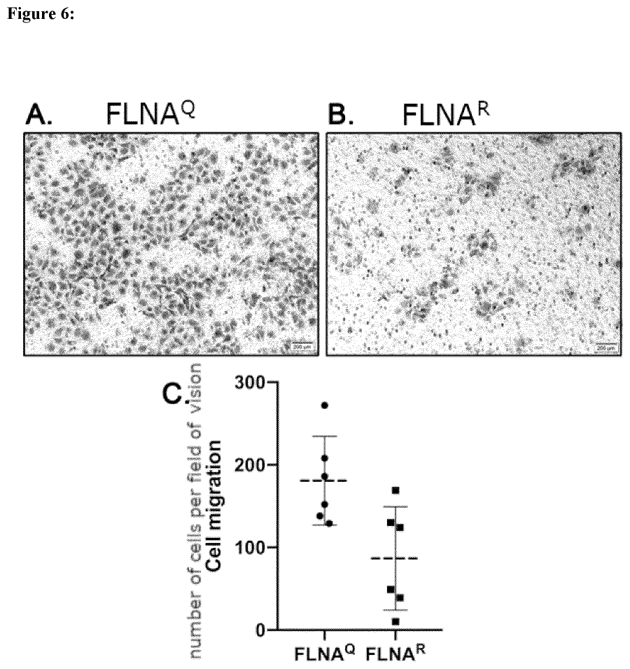

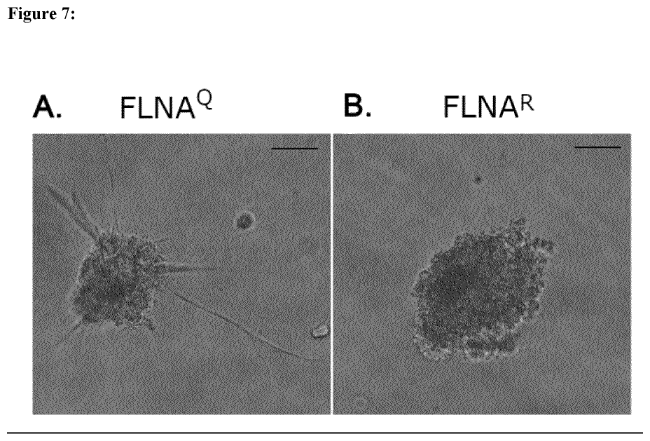

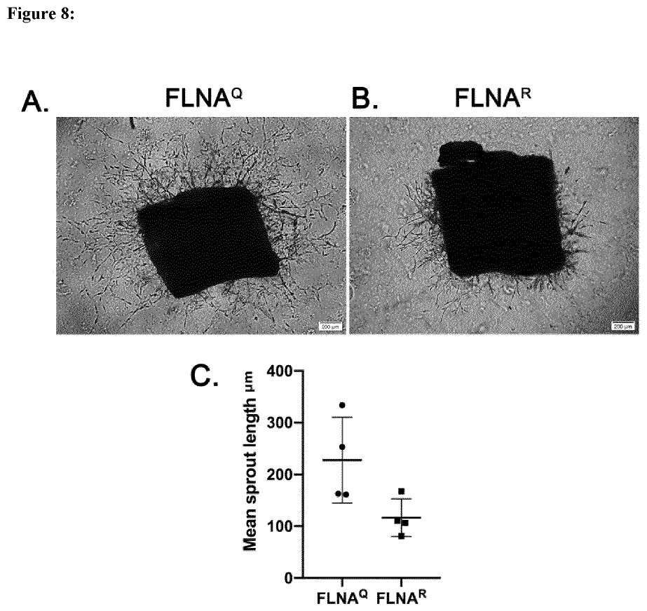

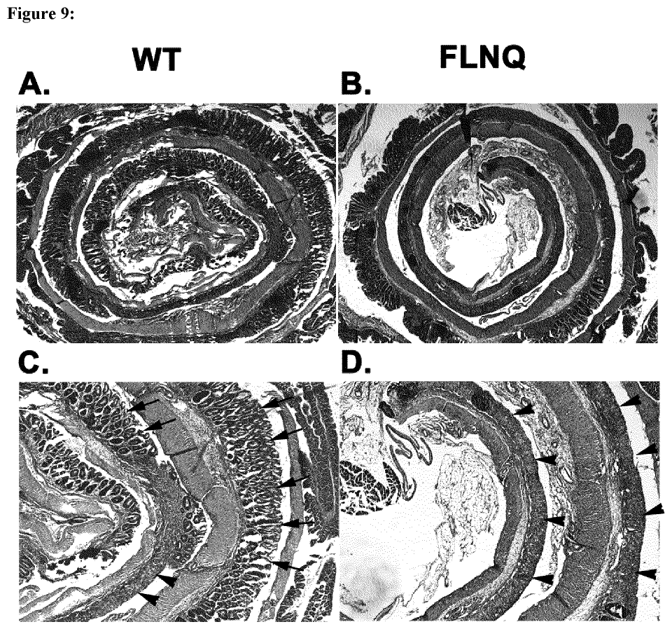

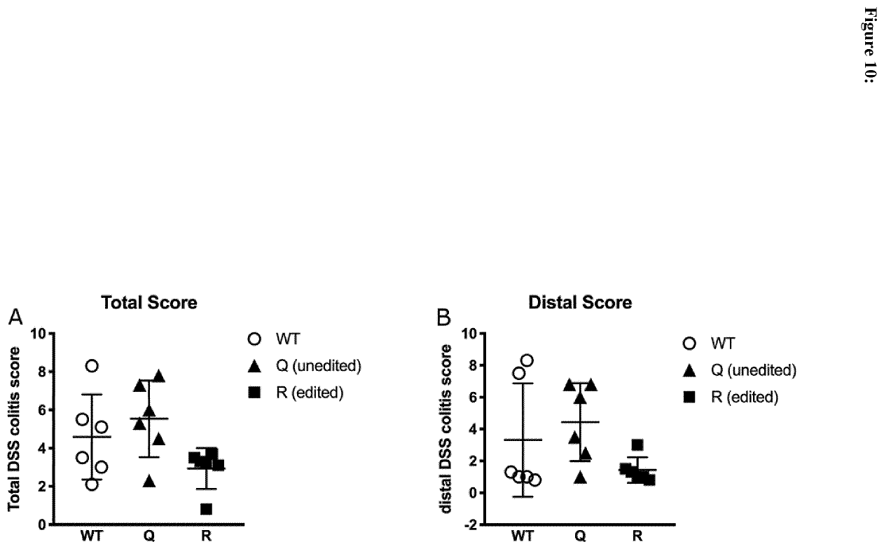

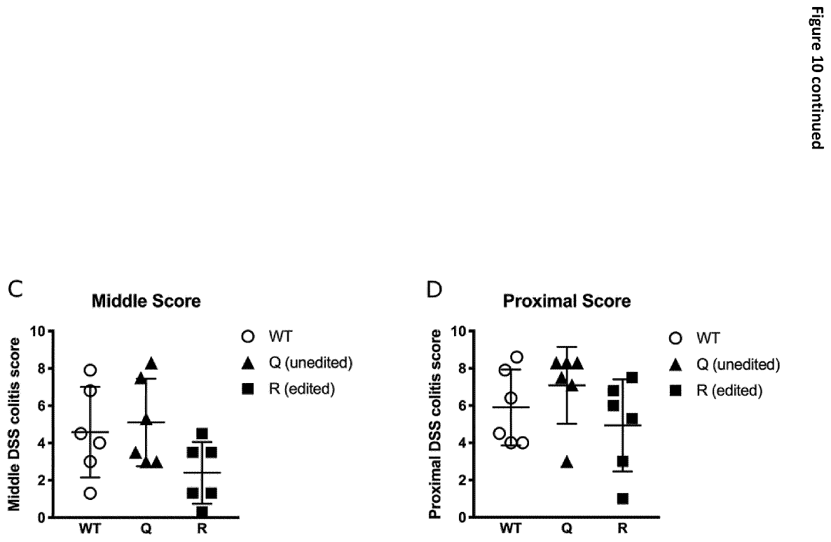

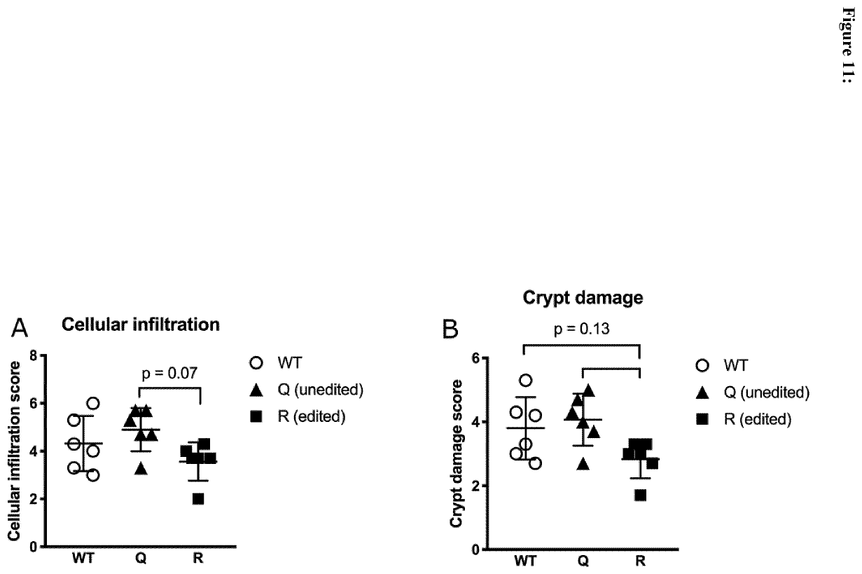

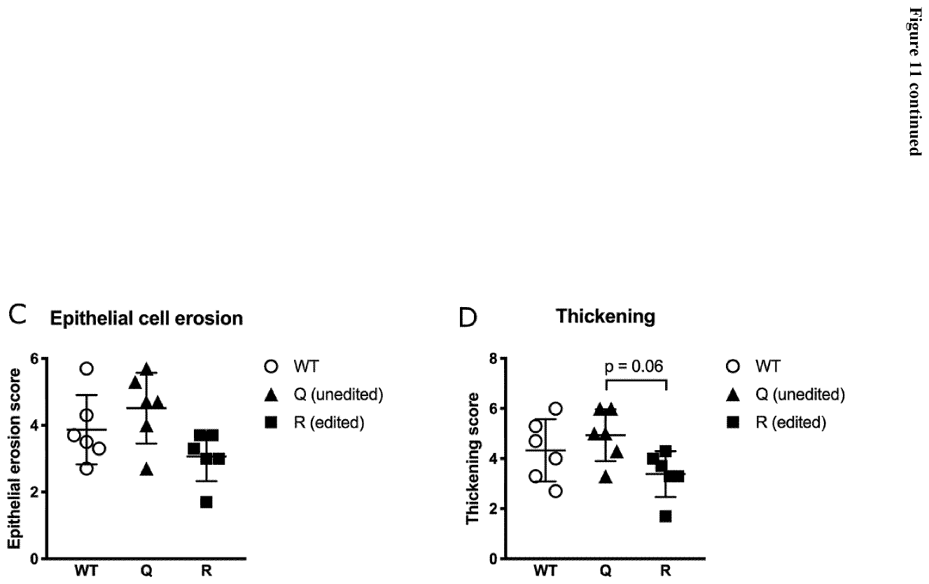

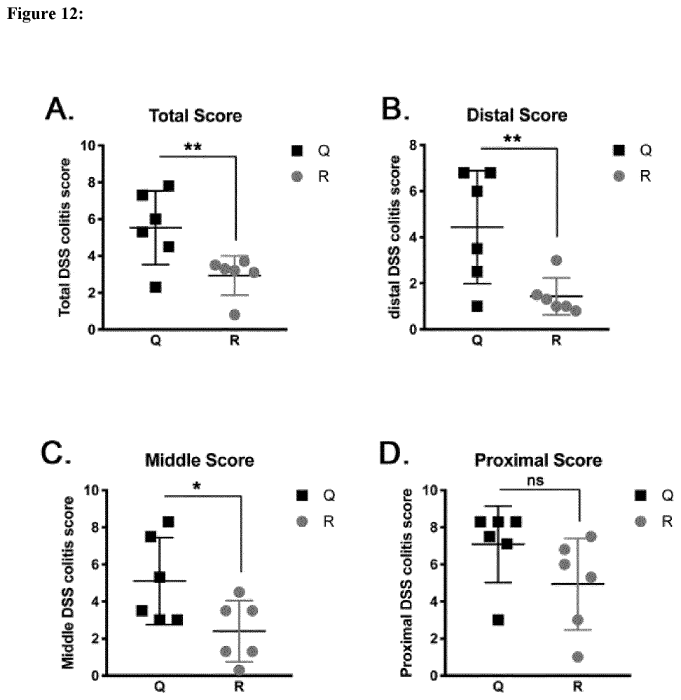

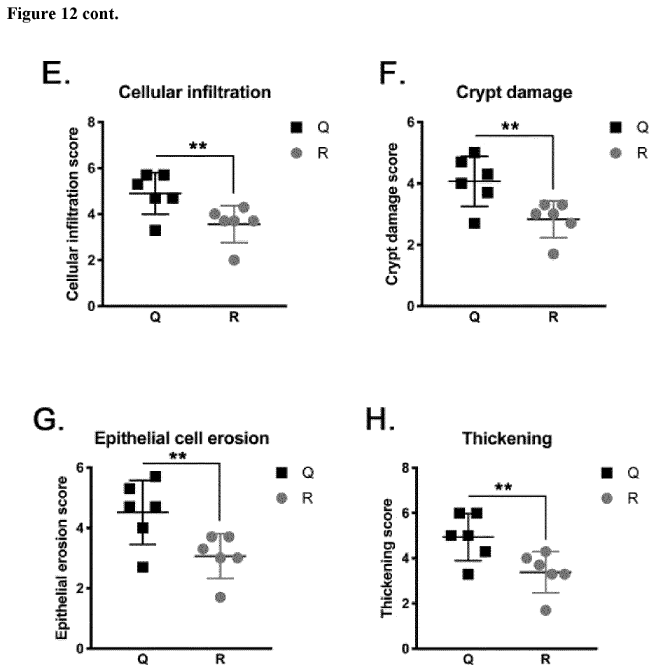

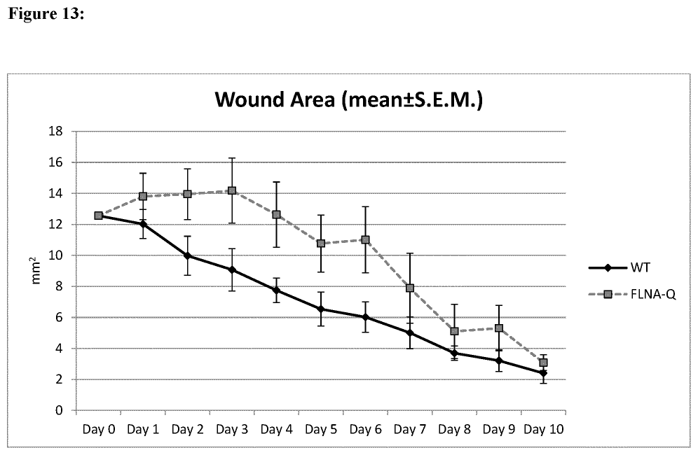

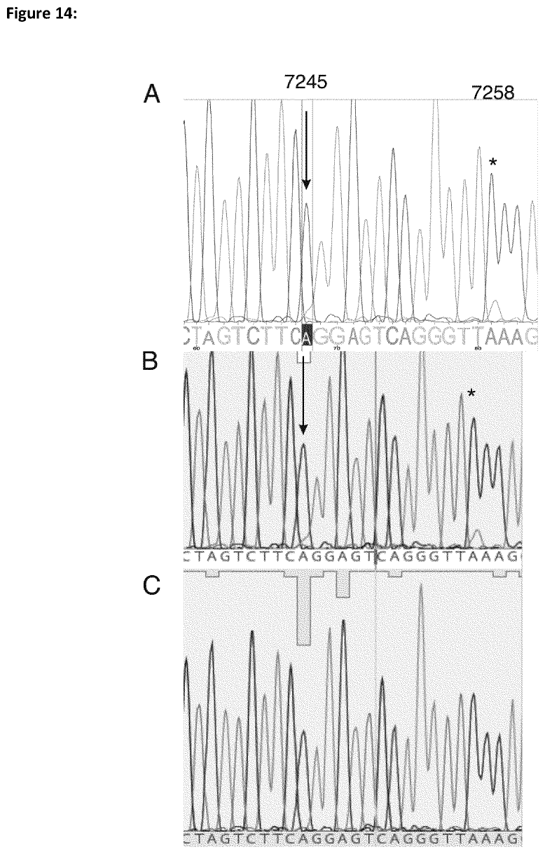

(SEQ ID NO: 41)
GCTACCTGGGTGTGTGATTGTGG

where the PAM sequences are underlined and boldface, see FIG. 1 for details. Subsequent, a homology directed repair (HDR) based template (SEQ ID NO: 49) was used to precisely delete the ECS region. The HDR also spans the edited region but carries a CGG codon replacing the to-be-edited CAG codon. Homologous recombination of this construct into the cut region converts the genomically encoded CAG to CGG (see FIG. 1B for sequence details). The repair construct and guide RNA Cas9 was injected into fertilized eggs. The fertilized eggs were inserted into pseudopregnant foster mothers. Offspring mice were tested for the FLNA^{R} allele by PCR using primers F2:

(SEQ ID NO: 44)
5′-GTCTGGGTGTTAGTCAGTTAGTC-3′

### and R2:

(SEQ ID NO: 45)
5′-CACTCTCCCATGCCACTATCTGC-3′.

Positive mice were identified by deletion of the intronic exon-complementary region (ECS) resulting in a PCR product reduced by 201 nt relative to wild-type. Conversion of CAG (Q) to CGG (R) was also verified by sanger sequencing by amplifying the converted region with primers F3:

(SEQ ID NO: 46)
5′-GTCTGGGTGTTAGTCAGTTAGTC-3′

### and R4:

(SEQ ID NO: 46)
5′-CGTCTACCCAGTACCAGATGTTG-3′.

homozygous by breeding. Subsequently mice were 6× backcrossed to C57BL6J mice to eliminate background mutations (FIG. 2). The resulting mice look normal at gross morphology and also breed normally and generate mice carrying the pre-edited filamin A allele at mendelian ratios.

### EXAMPLE 2: EFFECTS OF FLNA RNA EDITING ON TUMOR GROWTH AND TUMOR VASCULARIZATION

To test whether FLNA mRNA editing affects tumor growth, mice expressing FLNA variants constitutively edited or constitutively unedited were subjected to a tumor xenograft assay and the extent of vascularization in tumors was determined.

### Material and Methods

### Tumor Xenograft Assay

B16-F10 cells were cultured in DMEM medium supplemented with 10% fetal bovine serum. Cells were trypsinized and pelleted at 1200 rpm for 2 min. It is important to harvest cells in the log phase (50% confluent). The pellet was washed with 5 ml PBS and resuspended in 5 ml cold HBSS. The cell suspension was filtered through a cell strainer (70 μm) to remove clumps. Live cells were counted using Trypan blue. Viability should be >90%. The cell concentration was adjusted to 1×10⁶ cells/ml in ice-cold HBSS and cells were kept on ice until injected. The cells were injected as soon as possible. 100 μl (1×10⁵) cell suspension was injected per mouse subcutaneously above the hind limb using a 27 G needle. Injected mice were checked for the appearance of a bleb. If no bleb was observed, the mouse was discarded. Tumors were harvested 16 days post injection. After 16 days, tumor tissue was dissected and separated from the skin and then length (L), width (W) and height (H) of the tumor was measured with a caliper. The tumor volume was calculated by the formula π/6 (L×W×H). The tumor weight was determined as well. After measurements tumors were snap frozen for cryosectioning using Isopentane in liquid nitrogen and stored at −80° C. for subsequent experiments.

### Tumor Angiogenesis Assay

At least 4 tumors of each genotype were processed for cryosectioning. Multiple sections from each tumor were stained with CD31 antibody, a classical marker of endothelial cells. 10 μm sections were cut from every tumor tissue on a cryostat and tumor sections were used for immunostaining. Sections were thawed at room temperature (RT) and washed twice with 1× PBS. Sections were fixed with ice-cold methanol for 15 min at −20° C., followed by ice-cold acetone for 5 min at −20° C. After two washes with PBS the tumor tissue was blocked with 5% BSA for 30 min at RT. Sections were incubated with anti-CD31 (BD, catalog no. 557355) primary antibody (1:50) diluted in 3% BSA overnight at 4° C. in a humidified chamber. Next day the sections were washed twice with 1×PBS to remove the unbound primary antibody. Sections were incubated with secondary antibody (goat anti-Rat, Alexa 488, Invitrogen, catalog no. A-11006) diluted to 1:300 in 3% BSA for 1 h at RT. After two washes with 1×PBS sections were mounted using Antifade+DAPI and imaged at 20× magnification using a slide scanner (Olympus, BX61VS). The whole tumor scan was exported into several TIFF files. Exported files were opened in ImageJ and number of vessels per image was counted. The number of total vessels per tumor was calculated by summing up the number of vessels from each section. In the end, the vessel density (no. of vessels/mm²) was calculated for every tumor sample. Multiple sections from 4 tumors each of edited (FLNA^{Q}) and unedited (FLNA^{R}) mice were used for the analysis.

### Isolation of Endothelial Cells

Lungs were isolated and dissected from 5-6 week old mice and homogenized using 0.5 mg/ml Collagenase type I (SIGMA). The resulting cell suspension was bound to 50 μl ProteinG dynabeads (Thermo scientific) coupled with 2.5 μg CD31 antibody (BD biosciences) for 1 hr at 4° C. The beads were washed 3 times with medium and cells were plated on 10 cm diameter fibronectin coated dishes. Media was changed every 48 h until cells were confluent and were FACS sorted using ICAM2-488 (Thermo scientific) to enrich for endothelial cells. The FACS sorted cells were directly used for the experiments.

### Endothelial Migration Assay-Transwell Assay

8 μm, 24 well Transwell inserts (Corning) were coated with 10 μg/ml fibronectin for 1 h at 37° C. Then, 50,000 endothelial cells of each genotype were resuspended in DMEM+0.5% FBS and put on the upper chamber of the transwell insert. In the bottom chamber, 600 μl of the medium containing DMEM+0.5% FBS+VEGF (25 ng/ml) was placed. The transwell inserts were incubated for 16 h at 37° C. After 16 h, the non-migrated cells from the top chamber was removed using a wet cotton swab and the migrated cells were then stained with crystal violet and counted under a microscope.

### In Vitro 3D Sprouting Assay

2000 FACS sorted endothelial cells were plated in non-tissue culture U-bottom 96 well dish along with 5 mg/ml methylcellulose to generate 3D spheroids. After 24 h, the 3D spheroids were pelleted and embedded in a collagen bed using Collagen type I (Gibco). Pictures of the sprouts were taken after 72 h.

### Aortic Ring Sprouting Assay

Thoracic aortas were dissected from 8 weeks old mice. Aortas were cleaned of surrounding fat and cut into 1 mm rings using a stereomicroscope and a scale ruler. 3 rings were used per mouse for the experiment. Wells of a 48 well plate were coated with 100 μl of matrigel (Corning) and incubated at 37° C. for 30 min. The aortic rings were gently placed on the matrigel and incubated at 37° C. for 5 min. Then 50 μl of matrigel overlay was applied on the rings and placed at 37° C. again for 40 min. Afterwards 250 μl of medium containing 20 ng/ml VEGF (Peprotech) was added on top of the rings. The rings were incubated at 37° C. and images were taken after 3-4 days. Aortic ring sprouting was quantified using an online microscopy analysis platform (wimasis.com).

### Results

Mice deficient in FLNA editing were generated, thus only expressing the unedited FLNA^{Q} protein. Recently, also mice only expressing a constitutively edited FLNA allele (FLNA^{R}) were generated. Both these mice strains appear healthy by gross morphological criteria. To test whether FLNA editing can impact neo vascularization and therefore affects tumor growth, B16F10 cells were injected subcutaneously (Overwijk (2011) Curr Protoc Immunol Chapter 20:Unit 20 1) in FLNA^{Q} (unedited) and FLNA^{R} (edited) mice. Two weeks later, tumors were harvested from these mice and tumor weight and size was measured and recorded. A striking difference in tumor size and weight was observed between the 2 genotypes (FIG. 3A). The quantifications show an average of 5-fold difference in tumor weight (FIG. 3B) and 6-fold difference in the tumor volume (FIG. 3C). These results show that the FLNA editing status of the host affects tumor growth in melanoma xenografts. The xenografts were further analyzed to determine the extent of vascularization in tumors grown in FLNA edited vs unedited mice. At least 4 tumors of each genotype were processed for cryosectioning. Multiple sections from each tumor were stained with CD31 antibody, a classical marker of endothelial cells and analyzed for vessel density in the tumor. Since the size of the tumors of edited vs unedited mice were dramatically different, the total vessel number was normalized by the size of the tumor to calculate the vessel density. The vessel density in the tumors grown in the edited (FLNA^{R}) mice was at least 2-fold lower than the one found in tumors grown in unedited FLNA^{Q} mice (FIG. 4). These results show that FLNA RNA editing regulates neo vascularization.

In context of the invention it is shown that an ADAR2 target, FLNA impacts the formation of tumor in a xenograft assay. In context of this invention, it is surprisingly shown that tumors growing in a host solely expressing edited FLNA grow much smaller indicating that FLNA editing acts as a gatekeeper in regulating tumor progression (FIG. 6). Tumorigenesis is a multistage dynamic process and involves initiation, progression and metastasis. The tumor microenvironment consists of many other cell types apart from tumor cells like Immune-inflammatory cells, growing blood vessels, other stromal cells like host-derived fibroblasts (Balkwill (2012) J Cell Sci 125(Pt 23):5591-6). The interaction between the tumor cells and the niche is extremely important for regulating tumor development and metastasis. We found that the vascular supply in the growing tumors inside a microenvironment containing edited FLNA^{R} is much reduced when compared to tumors grown in an environment solely expressing unedited FLNA^{Q}.

It is envisaged that edited endothelial cells are not able to move robustly toward the growing tumor and hence not able to efficiently form blood vessels. To test for differences in endothelial cell migration and sprouting, two assays were performed. On the one hand, isolated endothelial cells derived from mice of different genotypes (FLNA^{Q} or FLNA^{R}) were cultured in fibronectin-coated trans-well chambers. VEGF was added to the lower chamber as a chemoattractant. The number of cells that migrated from the upper to the lower chamber was determined after 16 h by fixing and staining cells in the lower chamber (FIG. 6). As can be seen clearly, endothelial cells expressing FLNA^{Q} migrate much faster towards the VEGF containing lower chamber, a situation that may also exist in growing solid tumors, that secrete VEGF.

To test for sprouting of endothelial cells in a more direct way, isolated endothelial cells were cultured in methylcellulose to form spheroids. Spheroids were embedded in type I collagen. Spontaneous sprouting was measured after 72 h (FIG. 7). A clear increase in sprouting was observed for FLNA^{Q} cells while almost no sprouting was observed for cells expressing exclusively FLNA^{R}. A similar result was obtained from an aortic sprouting assay. Aortic rings from mice expressing exclusively FLNA^{Q} or FLNA^{R} were incubated with VEGF and vessel sprouting was quantified after 3-4 days (FIG. 8). This indicates that FLNA^{Q} expressing endothelial cells have a higher propensity to form novel microvasculature than cells expressing FLNA^{R}.

Also, the role of infiltrating immune cells and other stromal associated cells cannot be dismissed to play a role in regulating FLNA editing associated differential tumor growth.

Whether the editing status of a tumor may also impact on its growth is currently unknown. Experiments testing the tumor editing status and its impact on growth and metastasis are currently under way. If this was the case, interfering with editing in tumors would provide a handle to regulate its growth and invasion into other tissues.

In contrast, in context of the present invention it is suggested that the editing status of the vasculature drives vascularization. Currently, VEGF inhibitors like bevacizumab provide a useful tool to curb tumor angiogenesis and hence its growth (Ranieri (2006) Curr Med Chem 13(16):1845-57). However, VEGF blockade induces hypoxia and secretion of angiogenic factors thus switching a more invasive scenario around the tumor. Modulating the FLNA RNA-editing status might provide an easy to use tool to inhibit angiogenesis in the host tissue surrounding the tumor. Manipulation of FLNA editing may be achieved by site-directed editing (Montiel-Gonzalez (2019) Methods 156:16-24). Alternatively, interfering with FLNA protein function by modulating its expression or biochemical and biophysical activity may provide another useful mechanism by which neo-vascularization and cell growth may be inhibited.

### EXAMPLE 3: EFFECTS OF FLNA RNA EDITING ON DSS INDUCED COLITIS

Since RNA editing is highest not only in the vasculature but also in the large intestine, the role of FLNA RNA editing in gastrointestinal integrity and inflammatory bowel diseases was investigated. To test whether FLNA RNA editing can impact colon inflammation, FLNA^{Q} and FLNA^{R} mice were challenged with dextran sodium sulfate (DSS) and subsequently the colon was analysed.

11 weeks old (6 mice/genotype) wild type (wt), unedited (FLNA^{Q}) and constitutively edited (FLNA^{R}) mice created as described in Example 1 were treated orally with 2% DSS for 7 days and then fed with normal water for 3 days for the recovery. After total of 10 days, mice were sacrificed and their colons were dissected. The colons were cleaned, cut longitudinally, rolled using a toothpick and placed on a small filter paper. The “swiss rolls” were then fixed in 4% paraformaldehyde overnight and processed for paraffin sectioning. 5 μm sections were taken from each sample using microtome and stained with Hematoxylin and Eosin. The stained slides were imaged and blinded samples were scored manually by a trained technical assistant following a pre-defined scoring scheme (Koelink (2018) Journal of Crohn's & colitis 12:794-803). The severity of DSS induced colitis was scored at a scale of 0-10 depending on the extent of inflammation. The total score was determined from the whole colon and proximal, middle and distal score was determined across different parts of the colon. The distal score was measured near rectum and proximal score was measured near the caecum. Various colitis parameters were analyzed like cellular infiltration, crypt damage, epithelial cell erosion and intestinal thickening to further grade the magnitude of developing colitis (Koelink (2018) loc. cit.). For statistical analysis, one-way analysis of variance (ANOVA) analysis was performed to compare the 3 genotypes across each parameter. In order to compare FLNA^{Q} and FLNA^{R} samples directly, a t-test was performed. P<0.05 was considered statistically significant.

Analysis of colon sections from DSS-treated mice showed that FLNA^{Q} mice displayed increased loss of intestinal crypts (crypt damage) and increased loss of epithelial cells (epithelial erosion) as compared to the wild type colon sections (FIG. 9). The colitis manifestation was scored across all samples and fully edited mice (FLNA^{R}) showed a clear resistance towards DSS induced colitis as compared to either FLNA^{Q} or wt mice (FIG. 10A). The total scoring as well as scoring across different parts of colon (distal, middle and proximal) showed a similar pattern (FIG. 10 B-D). These results show that FLNA editing in the host could prevent colon inflammation under a challenged condition. Several parameters characterizing a colitis condition were also measured in the colon sections. Epithelial cell erosion and crypt damage was significantly reduced in FLNA^{R} mice as compared to FLNA^{Q} (FIG. 11B, C). Inflammation parameters like lymphocyte infiltration and thickening of the intestinal wall—all parameters indicative of inflammatory processes—showed a trend towards reduction in FLNA^{R} mice when compared to unedited mice (FIG. 11A, D). Most of the parameters remained unchanged when compared between wt and FLNA^{Q} mice (FIG. 10). These results show that FLNA editing has a significant impact on DSS induced colitis. We also compared the colitis-associated parameters directly between FLNA^{Q} and FLNA^{R} mice and checked for significant differences using a t-test. The total colitis scoring suggests that the colitis-induced inflammation was significantly reduced in mice expressing fully edited FLNA^{R} when compared to unedited mice (FIG. 12A). Moreover, the colitis progression was remarkably reduced at the distal (near rectum) and middle part of the colon in FLNA^{R} mice. However, colitis progression seemed very similar at the proximal side (near caecum) between FLNA^{Q} and FLNA^{R} mice (FIG. 12B-D). Similarly, when the characteristic features associated with DSS induced colitis were analyzed directly between FLNA^{Q} and FLNA^{R} mice using a t-test, all parameters including cellular infiltration, crypt damage, epithelial cell erosion and thickening were dramatically reduced in FLNA^{R} mice as compared to FLNA^{Q} mice (FIG. 12E-H). These results show that FLNA editing can protect against colon inflammation. Furthermore, the results show that mice expressing edited FLNA are less susceptible to DSS-induced colitis as compared to unedited mice indicating that editing of FLNA mRNA can regulate the colon inflammation under a diseased condition. DSS is a water soluble, anticoagulant compound that is toxic to the intestinal epithelium (Chassaing (2014) Curr Protoc Immunol 104:Unit 15 25). The mechanism behind DSS induced colitis in not entirely clear. However, it will be interesting to test whether the editing status of FLNA can regulate mucin expression and/or intestinal epithelial barrier integrity after DSS treatment which is the first line of defense in intestinal disorders. Indeed, preliminary data from the inventor indicate that mucin expression is altered in FLNA^{Q} and FLNA^{R} mice (Jantsch lab, unpublished). IBDs are characterized by chronic intestinal inflammation due to aberrant immune responses in the genetically predisposed host. Without being bound by theory, it is envisaged that unedited FLNA stimulates production of pro-inflammatory cytokines causing enhanced colon inflammation after DSS treatment. Previous data from the inventor show that FLNA editing is highly dynamic along the course of colitis progression, high in the untreated colon, reduces after DSS induced colitis and again restored upon recovery, corroborating a direct relationship between ongoing inflammation and FLNA editing. The dynamic change in FLNA editing levels during colitis suggests implications in regulating intestinal homeostasis. It is also shown that wild type mice have higher FLNA editing in the distal part of colon as compared to the proximal part. The current results show that unedited mice are more sensitive to DSS induced colitis in the distal part of the colon than in the proximal part. These results indicate that high FLNA editing in the distal part of colon might be required to protect from colon inflammation associated disorders.

The intestinal epithelial barrier layer has a high turnover and is regulated by gut microbiota, diet, host immunity and environmental factors. Intestinal microbiota has been shown to play an important role in colon inflammation (Chassaing (2011) Gastroenterology 140(6):1720-28). The analysis of differences in microbiota of FLNA^{Q} vs FLNA^{R} mice is initiated to determine whether FLNA editing affects the balance of the gut microbiome. Ongoing studies also aim at determining the microbiota-epithelial cross talk to shed more light into the mechanism by which FLNA editing can regulate the manifestation of DSS-induced colitis. Differential gene expression analysis of colons from untreated wildtype and FLNA^{Q} mice showed overexpression of β-defensins (data not shown). Altered function of the antimicrobial peptides (0-defensins) has been implicated in IBD progression (Cobo Pathogens 2(1):177-92). Further gene expression analysis of DSS treated FLNA^{Q} vs FLNA^{R} colons would be useful in understanding the mechanism behind this regulation. The DSS-induced colitis model has some shortcomings. Still it remains a reliable model to study inflammatory bowel diseases. It is shown that FLNA editing has a significant impact on induced colitis. Further mechanistic studies will help to understand the interactions between host genetics, gut innate immunity and microbiota. Clearly, given that FLNA and FLNB editing is regulated by intramolecular structures formed within the pre-mRNA opens the possibility to manipulate editing within these two pre-mRNAs for therapeutic purposes. This might be achieved by antisense technologies to directly target the editing site but also by interfering with the splicing reaction that limits the time at which the editing site remains available (Montiel-Gonzalez (2019) loc. cit.; Licht (2019) loc. cit.). In any case, manipulating editing to increase the editing state of FLNA but also to alter its expression and thus interaction with interacting proteins may provide a tool to protect against or reduce the extent of inflammatory bowel diseases.

### EXAMPLE 4: IMPACT OF FLNA EDITING ON WOUND HEALING

Filamin A is an actin-crosslinking protein and it was shown that Filamin A pre-mRNA editing affects the contractile cell apparatus (Jain (2018) loc. cit.) The effect of Filamin A editing on cell migration was therefore tested using a wound healing assay. This was performed by inserting a 4 mm diameter whole on the shaved back of a narcotized mouse using a sterile disposable biopsy puncher. Immediately after generating the wound the diameter is measured using a caliper. Wound closure is measured for 10 days using a caliper (FIG. 13). Using this method, a delay in wound healing is observed in FLNA^{Q} mice compared to wild-type mice.

### EXAMPLE 5: IN VIVO EDITING OF THE ENDOGENOUS MOUSE FILAMIN A MRNA IN MOUSE 3T3 CELLS

In the following experiment it is shown that a ONC can induce site-specific RNA editing in vivo. NIH 3T3 cells were transfected with a ONC, cDNA was generated from isolated mRNA and the editing status of Filamin A mRNA was determined.

### Cell Culture and Transfection

NIH 3T3 cells were cultured in Dulbecco's Minimal Essential Medium (DMEM) complemented with 10% fetal calf serum, 1% penicillin/streptomycin and 2 mM L-glutamine.

For transfection, cells were seeded at ˜ 30% confluency (1×10⁶) cells on a 6 cm tissue culture dish. After overnight incubation in a tissue culture incubator in a 5% C02 atmosphere, cells were transfected with 5 μg of ONC per plate. The used ONC is complementary to exon 42 and 43 of mouse Filamin A mRNA (SEQ ID NO: 50) and has the sequence of SEQ ID NO: 51 and the modifications as shown below. The oligonucleotide was synthesized by IDT Integrated DNA Technologies, BVBA (Interleuvenlaan 12A, B-3001 Leuven, Belgium) and had the sequence
mA*mA*mA*GmA*rUmGrCrUrGrGrCrUrGmGrUrUmGrArCrCrUmUrUrArAmCrCrCmUr GrArCrUCCCGrArArGrArCrUmArGmA*mA*mA*mC-3′ (SEQ ID NO: 51),
where
A is desoxyadenosine,
G is desoxyguanosine,
C is desoxycytosine,
T is desoxythymidine,
stands for phosphothioate linkage,
m stands for 2′ O-methyl ribose,
rU is uridine,
rA is adenosine,
rC is cytidine, and
rG is guanosine.

For transfection 250 μl of Opti-MEM medium (Life technologies) was mixed with 10 μl of Lipofectamine 3000 reagent (Life technologies). Also 250 μl of Opti-MEM medium were mixed with 5 μg of above described oligonucleotide and 10 μl of P3000 reagent (Life technologies). Subsequently the 260 μl of Lipofectamine 3000 diluted in OptiMEM were mixed with the 260 μl of RNA oligonucleotide diluted in Opti-MEM and P3000 reagent. Both components were mixed and incubated for 5 minutes at room temperature before the entire 500 μl were added to the 3T3 cells from overnight incubation.

The cells and the transfection reagent were incubated over night (18 h) in a tissue culture incubator supplemented with 5% C02. Subsequently, the medium was removed and the cells were incubated with fresh medium (6 ml, DMEM supplemented with 1% Pen/Strep, 10% Fetal calf serum, 2 mM L-Glutamine).

### RNA Isolation

After incubation for an additional 48 h, the culture medium was removed and total RNA was isolated using Trizol reagent (Thermofisher, Waltham, MA). After removal of the culture medium the adherent cells were washed once with ice-cold 1× PBS (5 ml per 6 cm dish). After removal of 1×PBS 500 μl Trizol reagent was added to the cells and the cells were lysed and removed from the plate by thorough rinsing with Trizol. The lysed cell extract was transferred to an Eppendorf tube and 400 μl of chloroform were added. After thorough vortexing for 2 minutes, the lysate was centrifuged in an Eppendorf centrifuge at 13.000 rpm for 10 minutes. The aqueous supernatant was transferred to a fresh Eppendorf centrifuge tube and 300 μl of Phenol/Chloroform (25:25) mixture was added. After thorough vortexing for 2 minutes, the lysate was centrifuged in an Eppendorf centrifuge at 13.000 rpm for 10 minutes. The aqueous supernatant was transferred to a fresh Eppendorf centrifuge tube and 300 μl of Chloroform was added. After thorough vortexing for 2 minutes, the lysate was centrifuged in an Eppendorf centrifuge at 13.000 rpm for 10 minutes. The aqueous supernatant was transferred to a fresh Eppendorf centrifuge tube. The RNA was precipitated by addition of 500 μl isopropanol and thorough mixing. The RNA was collected by centrifugation in an Eppendorf centrifuge at 13.000 rpm for 10 minutes. The supernatant was removed and the pellet was air dried for 10 minutes at 37° C.

The RNA pellet was resuspended in 100 μl of deionized and sterilized H₂O. Contaminating DNA was removed by digestion with 10 Units of RNAse-free DNAse (New England Biolabs, Ipswich, MA) for 15 minutes at 37°.

The remaining RNA was purified by extraction with phenol and chloroform. To do so 50 μl of Phenol/Chloroform mixture are added. After thorough vortexing for 2 minutes, the lysate was centrifuged in an Eppendorf centrifuge at 13.000 rpm for 10 minutes. The aqueous supernatant was transferred to a fresh Eppendorf centrifuge tube and 50 μl of Chloroform was added. After thorough vortexing for 2 minutes, the lysate was centrifuged in an Eppendorf centrifuge at 13.000 rpm for 10 minutes. The aqueous supernatant was transferred to a fresh Eppendorf centrifuge tube. The RNA was precipitated by addition of 250 μl ethanol (96%) and 25 μl of 3M NaAcetate pH 5.2 and thorough mixing. The RNA was collected by centrifugation in an Eppendorf centrifuge at 13.000 rpm for 10 minutes, the pellet was washed with 70% Ethanol. The supernatant was removed and the pellet was allowed to air dry for 10 minutes at 37° C.

cDNA Synthesis

For cDNA synthesis the RNA was resuspended in 20 μl of nuclease-free water. 3 μl (˜1 μg) of RNA were used for a cDNA synthesis by adding 2 μl (200 ng) random hexamers (New England Biolabs) and 11 μl of nuclease free water. The mixture was incubated for 3 minutes at 70° C. and then put on ice. Subsequently, 2 μl of a mixture of 5 mM each dATP, dCTP, dGTP, dTTP, 1 μl of RNASE inhibitor (New England Biolab), and 1 μl MMTV reverse transcriptase was added and the mixture was incubated at 37° C. for 90 minutes. The mixture was subsequently stored frozen at −20° C.

### PCR

For PCR of the region spanning exon 42 and 43 covering the editing site the following primers was used:
Primer forward:

(SEQ ID NO: 52)
GTCAAGTTCAACGAGGAGCAC

Primer reverse:

(SEQ ID NO: 53)
GTGCACCTTGGCATCAATTGC

The PCR was set up by using
2 μl of the first strand cDNA synthesis
10 ng forward primer
10 ng reverse primer
5 μl PCR buffer 5× concentrated (New England Biolabs)
1.5 μl 10 mM dNTP mixture
10 U Taq Polymerase (New England Biolabs)
Nuclease free water to 25 μl final volume.

The PCR mixture was incubated in a PCR Machine (BioRad T100) and the edited region was amplified by using the following temperature cycle:
5 min 95° C.
35 cycles of
40 seconds 95° C.
30 seconds 54° C.
30 seconds 72° C.
Followed by 5 minutes at 72° C.

### Gel Extraction

The amplified fragment was separated on a 1.2% agarose gel using a Tris acetate buffer system at 70 volts for 1 hr. The DNA was visualized by staining with ethidium bromide at 50 ng/ml and illumination at 280 nm.

The amplified product of ˜190 nucleotides was purified from a gel using a MonArch Gel extraction kit following the manufacturer's instruction (New England Biolabs).

### Sequencing

The extracted DNA fragment was validated and editing was determined by sanger sequencing using DNA primer)

(SEQ ID NO: 52)
GTCAAGTTCAACGAGGAGCAC

Using the Eurofins Genomics Mix2 Seq service (Eurofins Genomics Germany GmbH, Anzinger Str. 7a, 85560 Ebersberg, Germany)

Resulting chromatograms were evaluated using the Geneious sequence analysis software (Geneious, Biomatters, Ltd., L2, 18 Shortland Street, Auckland, 1010, New Zealand).

### Results

The sequencing chromatogram demonstrates that the described ONC was successfully transferred in the cells and induced RNA editing. Position 7245 in mouse Filamin A corresponding to position 7247 of SEQ ID NO: 7 was edited when cells were transfected with the ONC. The editing event lead to conversion of adenosine to inosine. Inosine is detected as guanosine during sequencing. Both a “guanosine-peak” and an “adenosine-peak” were detected at position 7245 since not all mRNA molecules were edited due to the nature of the cells based assay.

1. A pharmaceutical composition comprising a modified Filamin A encoded by a nucleic acid molecule, wherein the nucleic acid molecule is characterized in that the codon in the wildtype sequence encoding glutamine corresponding to position 2333 of SEQ ID NO: 27 is replaced by a codon encoding arginine and/or comprising a modified Filamin B encoded by a nucleic acid molecule, wherein the nucleic acid molecule is characterized in that the codon in the wildtype sequence encoding glutamine corresponding to position 2327 of SEQ ID NO: 29 is replaced by a codon encoding arginine.

2. The pharmaceutical composition according to claim 1, wherein the modified Filamin A has an actin-binding domain and an arginine corresponding to the arginine at position 2333 of SEQ ID NO: 1 and is encoded by a nucleic acid molecule selected from the group consisting of
(i) a nucleic acid molecule encoding a polypeptide comprising an amino sequence as depicted in SEQ ID NOs: 1 or 3;
(ii) a nucleic acid molecule comprising a nucleotide sequence as depicted in SEQ ID NOs: 2 or 4;
(iii) a nucleic acid molecule hybridizing under stringent conditions to the complementary strand of the nucleic acid molecule of (i) to (ii);
(iv) a nucleic acid molecule having a nucleotide sequence with at least 60% sequence identity to the nucleotide sequence of a nucleic acid molecule of any one of (i) to (iii); and
(v) a nucleic acid molecule comprising a nucleotide sequence which is degenerate as a result of the genetic code to the nucleotide sequence of a nucleic acid molecule of any one of (i) to (iv);
and/or wherein the modified Filamin B has an actin-binding domain and an arginine corresponding to the arginine at position 2327 of SEQ ID NO: 5 and is encoded by a nucleic acid molecule selected from the group consisting of
(a) a nucleic acid molecule encoding a polypeptide comprising an amino sequence as depicted in SEQ ID NO: 5;
(b) a nucleic acid molecule comprising a nucleotide sequence as depicted in SEQ ID NO: 6;
(c) a nucleic acid molecule hybridizing under stringent conditions to the complementary strand of a nucleic acid molecule of (a) or (b);
(d) a nucleic acid molecule having a nucleotide sequence with at least 60% sequence identity to the nucleotide sequence of a nucleic acid molecule of any one of (a) to (c); and
(e) a nucleic acid molecule comprising a nucleotide sequence which is degenerate as a result of the genetic code to the nucleotide sequence of a nucleic acid molecule of any one of (a) to (d).

3-8. (canceled)

9. A pharmaceutical composition comprising a nucleic acid molecule encoding a modified Filamin A of claim 1, wherein said nucleic acid molecule is selected from the group consisting of:
(i) a nucleic acid molecule comprising the nucleotide sequence as depicted in SEQ ID NOs: 2 and 4;
(ii) a nucleic acid molecule hybridizing under stringent conditions to the complementary strand of a nucleic acid molecule of (i);
(iii) a nucleic acid molecule having a nucleotide sequence with at least 60% sequence identity to the nucleotide sequence of a nucleic acid molecule of (i) or (ii); and
(iv) a nucleic acid molecule comprising a nucleotide sequence which is degenerate as a result of the genetic code to the nucleotide sequence of a nucleic acid molecule of any one of (i) to (iii);
wherein said modified Filamin A has an actin-binding domain and an arginine corresponding to the arginine at position 2333 of SEQ ID NO: 1;
and/or comprising a modified Filamin B as defined in claims 1 or 2, wherein said nucleic acid molecule is selected from the group consisting of:
(a) a nucleic acid molecule comprising the nucleotide sequence as depicted in SEQ ID NO: 6;
(b) a nucleic acid molecule hybridizing under stringent conditions to the complementary strand of a nucleic acid molecule of (a);
(c) a nucleic acid molecule having a nucleotide sequence with at least 60% sequence identity to the nucleotide sequence of a nucleic acid molecule of (a) or (b); and
(d) a nucleic acid molecule comprising a nucleotide sequence which is degenerate as a result of the genetic code to the nucleotide sequence of a nucleic acid molecule of any one of (a) to (c);
wherein said modified Filamin B has an actin-binding domain and an arginine corresponding to the arginine at position 2327 of SEQ ID NO: 5.

10. A pharmaceutical composition comprising a vector comprising the nucleic acid(s) as defined in claim 9.

11. A pharmaceutical composition comprising an oligonucleotide construct capable of modifying Filamin A RNA, wherein said modification is generated by site-specific RNA editing and comprises the conversion of adenosine corresponding to position 7247 of SEQ ID NO: 7 to inosine and/or capable of modifying Filamin B RNA, wherein said modification is generated by site-specific RNA editing and comprises the conversion of adenosine corresponding to position 7123 of SEQ ID NO: 9 to inosine.

12. The pharmaceutical composition of claim 11, wherein the oligonucleotide construct is partially complementary to sequences SEQ ID NO: 7, SEQ ID NO: 8 or SEQ ID NO: 9 and provides structures upon base pairing with the target sequence that allow recruiting of an RNA editing entity naturally present in the cell or provided together with the oligonucleotide construct and capable of performing the editing of the nucleotide.

13. The pharmaceutical composition according to claim 11, wherein the oligonucleotide construct for modification of Filamin A RNA comprises or consists of a sequence identical or at least 60% identical to a nucleotide sequence selected from the group consisting of SEQ ID NOs: 10 to 13 and 56.

14. The pharmaceutical composition according to claim 11, wherein the oligonucleotide construct for modification of Filamin B RNA comprises or consists of a sequence identical or at least 60% identical to a nucleotide sequence selected from the group consisting of SEQ ID NOs: 14 to 17.

15. The pharmaceutical composition according to claim 11, wherein the oligonucleotide construct comprises
(a) a targeting portion, comprising a sequence complementary to part of the target RNA; and
(b) a recruiting portion capable of binding and recruiting an RNA editing entity naturally present in the cell or provided together with the oligonucleotide construct and capable of performing the editing of the nucleotide.

16-17. (canceled)

18. The pharmaceutical composition of claim 17, wherein the oligonucleotide construct for modification of Filamin A RNA comprises or consists of a sequence identical or at least 60% identical to a nucleotide sequence selected from the group consisting of SEQ ID NOs: 18 to 21.

19. The pharmaceutical composition of claim 17, wherein the oligonucleotide construct for modification of Filamin B RNA comprises or consists of a sequence identical or at least 60% identical to a nucleotide sequence selected from the group consisting of SEQ ID NOs: 22 to 25.

20. The pharmaceutical composition according to claim 12, wherein the editing entity is ADAR1 or ADAR2.

21-23. (canceled)

24. The pharmaceutical composition according to claim 11, wherein the oligonucleotide recruits to the target RNA an RNA editing entity not naturally present in the cell and capable of performing the editing of the nucleotide.

25. The pharmaceutical composition of claim 24, wherein the RNA editing entity not naturally present in the cell comprises an ADAR catalytic domain.

26. The pharmaceutical composition of claim 25, wherein the RNA editing entity not naturally present in the cell comprises an ADAR catalytic domain and a recruiting domain, wherein the recruiting domain associates with the oligonucleotide construct as defined in claim 24 and binds to the target RNA upon association with said oligonucleotide construct.

27. The pharmaceutical composition of claim 26, wherein the recruiting domain is selected from the group consisting of SNAP-tag, lambda N protein, MS2 coat protein, CAS13 and CIRTS.

28. The pharmaceutical composition of claim 27, wherein the RNA editing entity not naturally present in the cell comprises or consists of an amino acid sequence identical or at least 60% identical to a sequence selected from the group consisting of SEQ ID NOs: 30 to 33 and 36 to 39.

29. The pharmaceutical composition according to claim 11, wherein the oligonucleotide construct comprises or consists of RNA.

30. The pharmaceutical composition according to claim 11, wherein the oligonucleotide construct comprises at least one sugar modification.

31-44. (canceled)

45. A method of treatment of a disease comprising administering the polypeptide as defined in any one of claims 1 to 8, the nucleic acid as defined in claim 9, the vector as defined in claim 10, the oligonucleotide construct as defined in any one of claims 11 to 34, the RNA editing entity not naturally present in the cell as defined in any one of claims 24 to 28 and/or combinations thereof to a subject in need thereof.

46. The method of treatment of claim 45, wherein the disease is a proliferative disease, an inflammatory disease, or a skin or mucosa lesion.

47-50. (canceled)


SEQUENCE LISTING (56 sequences)

SEQ ID NO: 1 - protein, 2639 aa, Homo sapiens [sequence omitted]

SEQ ID NO: 2 - DNA, 7920 bp, Homo sapiens
  filamin A isoform 1 modified cDNA
atgagtagctcccactctcgggcgggccagagcgcagcaggcgcggctccgggcggcggcgtcgacacgcgggacgccgagatgccggccaccgagaaggacctggcggaggacgcgccgtggaagaagatccagcagaacactttcacgcgctggtgcaacgagcacctgaagtgcgtgagcaagcgcatcgccaacctgcagacggacctgagcgacgggctgcggcttatcgcgctgttggaggtgctcagccagaagaagatgcaccgcaagcacaaccagcggcccactttccgccaaatgcagcttgagaacgtgtcggtggcgctcgagttcctggaccgcgagagcatcaaactggtgtccatcgacagcaaggccatcgtggacgggaacctgaagctgatcctgggcctcatctggaccctgatcctgcactactccatctccatgcccatgtgggacgaggaggaggatgaggaggccaagaagcagacccccaagcagaggctcctgggctggatccagaacaagctgccgcagctgcccatcaccaacttcagccgggactggcagagcggccgggccctgggcgccctggtggacagctgtgccccgggcctgtgtcctgactgggactcttgggacgccagcaagcccgttaccaatgcgcgagaggccatgcagcaggcggatgactggctgggcatcccccaggtgatcacccccgaggagattgtggaccccaacgtggacgagcactctgtcatgacctacctgtcccagttccccaaggccaagctgaagccaggggctcccttgcggcccaaactgaacccgaagaaagcccgtgcctacgggccaggcatcgagcccacaggcaacatggtgaagaagcgggcagagttcactgtggagaccagaagtgctggccagggagaggtgctggtgtacgtggaggacccggccggacaccaggaggaggcaaaagtgaccgccaataacgacaagaaccgcaccttctccgtctggtacgtccccgaggtgacggggactcataaggttactgtgctctttgctggccagcacatcgccaagagccccttcgaggtgtacgtggataagtcacagggtgacgccagcaaagtgacagcccaaggtcccggcctggagcccagtggcaacatcgccaacaagaccacctactttgagatctttacggcaggagctggcacgggcgaggtcgaggttgtgatccaggaccccatgggacagaagggcacggtagagcctcagctggaggcccggggcgacagcacataccgctgcagctaccagcccaccatggagggcgtccacaccgtgcacgtcacgtttgccggcgtgcccatccctcgcagcccctacactgtcactgttggccaagcctgtaacccgagtgcctgccgggcggttggccggggcctccagcccaagggtgtgcgggtgaaggagacagctgacttcaaggtgtacacaaagggcgctggcagtggggagctgaaggtcaccgtgaagggccccaagggagaggagcgcgtgaagcagaaggacctgggggatggcgtgtatggcttcgagtattaccccatggtccctggaacctatatcgtcaccatcacgtggggtggtcagaacatcgggcgcagtcccttcgaagtgaaggtgggcaccgagtgtggcaatcagaaggtacgggcctggggccctgggctggagggcggcgtcgttggcaagtcagcagactttgtggtggaggctatcggggacgacgtgggcacgctgggcttctcggtggaagggccatcgcaggctaagatcgaatgtgacgacaagggcgacggctcctgtgatgtgcgctactggccgcaggaggctggcgagtatgccgttcacgtgctgtgcaacagcgaagacatccgcctcagccccttcatggctgacatccgtgacgcgccccaggacttccacccagacagggtgaaggcacgtgggcctggattggagaagacaggtgtggccgtcaacaagccagcagagttcacagtggatgccaagcacggtggcaaggccccacttcgggtccaagtccaggacaatgaaggctgccctgtggaggcgttggtcaaggacaacggcaatggcacttacagctgctcctacgtgcccaggaagccggtgaagcacacagccatggtgtcctggggaggcgtcagcatccccaacagccccttcagggtgaatgtgggagctggcagccaccccaacaaggtcaaagtatacggccccggagtagccaagacagggctcaaggcccacgagcccacctacttcactgtggactgcgccgaggctggccagggggacgtcagcatcggcatcaagtgtgcccctggagtggtaggccccgccgaagctgacatcgacttcgacatcatccgcaatgacaatgacaccttcacggtcaagtacacgccccggggggctggcagctacaccattatggtcctctttgctgaccaggccacgcccaccagccccatccgagtcaaggtggagccctctcatgacgccagtaaggtgaaggccgagggccctggcctcagtcgcactggtgtcgagcttggcaagcccacccacttcacagtaaatgccaaagctgctggcaaaggcaagctggacgtccagttctcaggactcaccaagggggatgcagtgcgagatgtggacatcatcgaccaccatgacaacacctacacagtcaagtacacgcctgtccagcagggtccagtaggcgtcaatgtcacttatggaggggatcccatccctaagagccctttctcagtggcagtatctccaagcctggacctcagcaagatcaaggtgtctggcctgggagagaaggtggacgttggcaaagaccaggagttcacagtcaaatcaaagggtgctggtggtcaaggcaaagtggcatccaagattgtgggcccctcgggtgcagcggtgccctgcaaggtggagccaggcctgggggctgacaacagtgtggtgcgcttcctgccccgtgaggaagggccctatgaggtggaggtgacctatgacggcgtgcccgtgcctggcagcccctttcctctggaagctgtggcccccaccaagcctagcaaggtgaaggcgtttgggccggggctgcagggaggcagtgcgggctcccccgcccgcttcaccatcgacaccaagggcgccggcacaggtggcctgggcctgacggtggagggcccctgtgaggcgcagctcgagtgcttggacaatggggatggcacatgttccgtgtcctacgtgcccaccgagcccggggactacaacatcaacatcctcttcgctgacacccacatccctggctccccattcaaggcccacgtggttccctgctttgacgcatccaaagtcaagtgctcaggccccgggctggagcgggccaccgctggggaggtgggccaattccaagtggactgctcgagcgcgggcagcgcggagctgaccattgagatctgctcggaggcggggcttccggccgaggtgtacatccaggaccacggtgatggcacgcacaccattacctacattcccctctgccccggggcctacaccgtcaccatcaagtacggcggccagcccgtgcccaacttccccagcaagctgcaggtggaacctgcggtggacacttccggtgtccagtgctatgggcctggtattgagggccagggtgtcttccgtgaggccaccactgagttcagtgtggacgcccgggctctgacacagaccggagggccgcacgtcaaggcccgtgtggccaacccctcaggcaacctgacggagacctacgttcaggaccgtggcgatggcatgtacaaagtggagtacacgccttacgaggagggactgcactccgtggacgtgacctatgacggcagtcccgtgcccagcagccccttccaggtgcccgtgaccgagggctgcgacccctcccgggtgcgtgtccacgggccaggcatccaaagtggcaccaccaacaagcccaacaagttcactgtggagaccaggggagctggcacgggcggcctgggcctggctgtagagggcccctccgaggccaagatgtcctgcatggataacaaggacggcagctgctcggtcgagtacatcccttatgaggctggcacctacagcctcaacgtcacctatggtggccatcaagtgccaggcagtcctttcaaggtccctgtgcatgatgtgacagatgcgtccaaggtcaagtgctctgggcccggcctgagcccaggcatggttcgtgccaacctccctcagtccttccaggtggacacaagcaaggctggtgtggccccattgcaggtcaaagtgcaagggcccaaaggcctggtggagccagtggacgtggtagacaacgctgatggcacccagaccgtcaattatgtgcccagccgagaagggccctacagcatctcagtactgtatggagatgaagaggtaccccggagccccttcaaggtcaaggtgctgcctactcatgatgccagcaaggtgaaggccagtggccccgggctcaacaccactggcgtgcctgccagcctgcccgtggagttcaccatcgatgcaaaggacgccggggagggcctgctggctgtccagatcacggatcccgaaggcaagccgaagaagacacacatccaagacaaccatgacggcacgtatacagtggcctacgtgccagacgtgacaggtcgctacaccatcctcatcaagtacggtggtgacgagatccccttctccccgtaccgcgtgcgtgccgtgcccaccggggacgccagcaagtgcactgtcacaggtgctggcatcggccccaccattcagattggggaggagacggtgatcactgtggacactaaggcggcaggcaaaggcaaagtgacgtgcaccgtgtgcacgcctgatggctcagaggtggatgtggacgtggtggagaatgaggacggcactttcgacatcttctacacggccccccagccgggcaaatacgtcatctgtgtgcgctttggtggcgagcacgtgcccaacagccccttccaagtgacggctctggctggggaccagccctcggtgcagccccctctacggtctcagcagctggccccacagtacacctacgcccagggcggccagcagacttgggccccggagaggcccctggtgggtgtcaatgggctggatgtgaccagcctgaggccctttgaccttgtcatccccttcaccatcaagaagggcgagatcacaggggaggttcggatgccctcaggcaaggtggcgcagcccaccatcactgacaacaaagacggcaccgtgaccgtgcggtatgcacccagcgaggctggcctgcacgagatggacatccgctatgacaacatgcacatcccaggaagccccttgcagttctatgtggattacgtcaactgtggccatgtcactgcctatgggcctggcctcacccatggagtagtgaacaagcctgccaccttcaccgtcaacaccaaggatgcaggagaggggggcctgtctctggccattgagggcccgtccaaagcagaaatcagctgcactgacaaccaggatgggacatgcagcgtgtcctacctgcctgtgctgccgggggactacagcattctagtcaagtacaatgaacagcacgtcccaggcagccccttcactgctcgggtcacaggtgacgactccatgcgtatgtcccacctaaaggtcggctctgctgccgacatccccatcaacatctcagagacggatctcagcctgctgacggccactgtggtcccgccctcgggccgggaggagccctgtttgctgaagcggctgcgtaatggccacgtggggatttcattcgtgcccaaggagacgggggagcacctggtgcatgtgaagaaaaatggccagcacgtggccagcagccccatcccggtggtgatcagccagtcggaaattggggatgccagtcgtgttcgggtctctggtcagggccttcacgaaggccacacctttgagcctgcagagtttatcattgatacccgcgatgcaggctatggtgggctcagcctgtccattgagggccccagcaaggtggacatcaacacagaggacctggaggacgggacgtgcagggtcacctactgccccacagagccaggcaactacatcatcaacatcaagtttgccgaccagcacgtgcctggcagccccttctctgtgaaggtgacaggcgagggccgggtgaaagagagcatcacccgcaggcgtcgggctccttcagtggccaacgttggtagtcattgtgacctcagcctgaaaatccctgaaattagcatccaggatatgacagcccaggtgaccagcccatcgggcaagacccatgaggccgagatcgtggaaggggagaaccacacctactgcatccgctttgttcccgctgagatgggcacacacacagtcagcgtgaagtacaagggccagcacgtgcctgggagccccttccagttcaccgtggggcccctaggggaagggggagcccacaaggtccgagctgggggccctggcctggagagagctgaagctggagtgccagccgaattcagtatctggacccgggaagctggtgctggaggcctggccattgctgtcgagggccccagcaaggctgagatctcttttgaggaccgcaaggacggctcctgtggtgtggcttatgtggtccaggagccaggtgactacgaagtctcagtcaagttcaacgaggaacacattcccgacagccccttcgtggtgcctgtggcttctccgtctggcgacgcccgccgcctcactgtttctagccttcgggagtcagggctaaaggtcaaccagccagcctcttttgcagtcagcctgaacggggccaagggggcgatcgatgccaaggtgcacagcccctcaggagccctggaggagtgctatgtcacagaaattgaccaagataagtatgctgtgcgcttcatccctcgggagaatggcgtttacctgattgacgtcaagttcaacggcacccacatccctggaagccccttcaagatccgagttggggagcctgggcatggaggggacccaggcttggtgtctgcttacggagcaggtctggaaggcggtgtcacagggaacccagctgagttcgtcgtgaacacgagcaatgcgggagctggtgccctgtcggtgaccattgacggcccctccaaggtgaagatggattgccaggagtgccctgagggctaccgcgtcacctatacccccatggcacctggcagctacctcatctccatcaagtacggcggcccctaccacattgggggcagccccttcaaggccaaagtcacaggcccccgtctcgtcagcaaccacagcctccacgagacatcatcagtgtttgtagactctctgaccaaggccacctgtgccccccagcatggggccccgggtcctgggcctgctgacgccagcaaggtggtggccaagggcctggggctgagcaaggcctacgtaggccagaagagcagcttcacagtagactgcagcaaagcaggcaacaacatgctgctggtgggggttcatggcccaaggaccccctgcgaggagatcctggtgaagcacgtgggcagccggctctacagcgtgtcctacctgctcaaggacaagggggagtacacactggtggtcaaatggggggacgagcacatcccaggcagcccctaccgcgttgtggtgccctga

SEQ ID NO: 3 - protein, 2647 aa, Homo sapiens
  filamin-A isoform 2 modified
MSSSHSRAGQSAAGAAPGGGVDTRDAEMPATEKDLAEDAPWKKIQQNTFTRWCNEHLKCVSKRIANLQTDLSDGLRLIALLEVLSQKKMHRKHNQRPTFRQMQLENVSVALEFLDRESIKLVSIDSKAIVDGNLKLILGLIWTLILHYSISMPMWDEEEDEEAKKQTPKQRLLGWIQNKLPQLPITNFSRDWQSGRALGALVDSCAPGLCPDWDSWDASKPVTNAREAMQQADDWLGIPQVITPEEIVDPNVDEHSVMTYLSQFPKAKLKPGAPLRPKLNPKKARAYGPGIEPTGNMVKKRAEFTVETRSAGQGEVLVYVEDPAGHQEEAKVTANNDKNRTFSVWYVPEVTGTHKVTVLFAGQHIAKSPFEVYVDKSQGDASKVTAQGPGLEPSGNIANKTTYFEIFTAGAGTGEVEVVIQDPMGQKGTVEPQLEARGDSTYRCSYQPTMEGVHTVHVTFAGVPIPRSPYTVTVGQACNPSACRAVGRGLQPKGVRVKETADFKVYTKGAGSGELKVTVKGPKGEERVKQKDLGDGVYGFEYYPMVPGTYIVTITWGGQNIGRSPFEVKVGTECGNQKVRAWGPGLEGGVVGKSADFVVEAIGDDVGTLGFSVEGPSQAKIECDDKGDGSCDVRYWPQEAGEYAVHVLCNSEDIRLSPFMADIRDAPQDFHPDRVKARGPGLEKTGVAVNKPAEFTVDAKHGGKAPLRVQVQDNEGCPVEALVKDNGNGTYSCSYVPRKPVKHTAMVSWGGVSIPNSPFRVNVGAGSHPNKVKVYGPGVAKTGLKAHEPTYFTVDCAEAGQGDVSIGIKCAPGVVGPAEADIDFDIIRNDNDTFTVKYTPRGAGSYTIMVLFADQATPTSPIRVKVEPSHDASKVKAEGPGLSRTGVELGKPTHFTVNAKAAGKGKLDVQFSGLTKGDAVRDVDIIDHHDNTYTVKYTPVQQGPVGVNVTYGGDPIPKSPFSVAVSPSLDLSKIKVSGLGEKVDVGKDQEFTVKSKGAGGQGKVASKIVGPSGAAVPCKVEPGLGADNSVVRFLPREEGPYEVEVTYDGVPVPGSPFPLEAVAPTKPSKVKAFGPGLQGGSAGSPARFTIDTKGAGTGGLGLTVEGPCEAQLECLDNGDGTCSVSYVPTEPGDYNINILFADTHIPGSPFKAHVVPCFDASKVKCSGPGLERATAGEVGQFQVDCSSAGSAELTIEICSEAGLPAEVYIQDHGDGTHTITYIPLCPGAYTVTIKYGGQPVPNFPSKLQVEPAVDTSGVQCYGPGIEGQGVFREATTEFSVDARALTQTGGPHVKARVANPSGNLTETYVQDRGDGMYKVEYTPYEEGLHSVDVTYDGSPVPSSPFQVPVTEGCDPSRVRVHGPGIQSGTTNKPNKFTVETRGAGTGGLGLAVEGPSEAKMSCMDNKDGSCSVEYIPYEAGTYSLNVTYGGHQVPGSPFKVPVHDVTDASKVKCSGPGLSPGMVRANLPQSFQVDTSKAGVAPLQVKVQGPKGLVEPVDVVDNADGTQTVNYVPSREGPYSISVLYGDEEVPRSPFKVKVLPTHDASKVKASGPGLNTTGVPASLPVEFTIDAKDAGEGLLAVQITDPEGKPKKTHIQDNHDGTYTVAYVPDVTGRYTILIKYGGDEIPFSPYRVRAVPTGDASKCTVTVSIGGHGLGAGIGPTIQIGEETVITVDTKAAGKGKVTCTVCTPDGSEVDVDVVENEDGTFDIFYTAPQPGKYVICVRFGGEHVPNSPFQVTALAGDQPSVQPPLRSQQLAPQYTYAQGGQQTWAPERPLVGVNGLDVTSLRPFDLVIPFTIKKGEITGEVRMPSGKVAQPTITDNKDGTVTVRYAPSEAGLHEMDIRYDNMHIPGSPLQFYVDYVNCGHVTAYGPGLTHGVVNKPATFTVNTKDAGEGGLSLAIEGPSKAEISCTDNQDGTCSVSYLPVLPGDYSILVKYNEQHVPGSPFTARVTGDDSMRMSHLKVGSAADIPINISETDLSLLTATVVPPSGREEPCLLKRLRNGHVGISFVPKETGEHLVHVKKNGQHVASSPIPVVISQSEIGDASRVRVSGQGLHEGHTFEPAEFIIDTRDAGYGGLSLSIEGPSKVDINTEDLEDGTCRVTYCPTEPGNYIINIKFADQHVPGSPFSVKVTGEGRVKESITRRRRAPSVANVGSHCDLSLKIPEISIQDMTAQVTSPSGKTHEAEIVEGENHTYCIRFVPAEMGTHTVSVKYKGQHVPGSPFQFTVGPLGEGGAHKVRAGGPGLERAEAGVPAEFSIWTREAGAGGLAIAVEGPSKAEISFEDRKDGSCGVAYVVQEPGDYEVSVKFNEEHIPDSPFVVPVASPSGDARRLTVSSLRESGLKVNQPASFAVSLNGAKGAIDAKVHSPSGALEECYVTEIDQDKYAVRFIPRENGVYLIDVKFNGTHIPGSPFKIRVGEPGHGGDPGLVSAYGAGLEGGVTGNPAEFVVNTSNAGAGALSVTIDGPSKVKMDCQECPEGYRVTYTPMAPGSYLISIKYGGPYHIGGSPFKAKVTGPRLVSNHSLHETSSVFVDSLTKATCAPQHGAPGPGPADASKVVAKGLGLSKAYVGQKSSFTVDCSKAGNNMLLVGVHGPRTPCEEILVKHVGSRLYSVSYLLKDKGEYTLVVKWGDEHIPGSPYRVVVP

SEQ ID NO: 4 - DNA, 7944 bp, Homo sapiens
  filamin A isoform 2 modified
atgagtagctcccactctcgggcgggccagagcgcagcaggcgcggctccgggcggcggcgtcgacacgcgggacgccgagatgccggccaccgagaaggacctggcggaggacgcgccgtggaagaagatccagcagaacactttcacgcgctggtgcaacgagcacctgaagtgcgtgagcaagcgcatcgccaacctgcagacggacctgagcgacgggctgcggcttatcgcgctgttggaggtgctcagccagaagaagatgcaccgcaagcacaaccagcggcccactttccgccaaatgcagcttgagaacgtgtcggtggcgctcgagttcctggaccgcgagagcatcaaactggtgtccatcgacagcaaggccatcgtggacgggaacctgaagctgatcctgggcctcatctggaccctgatcctgcactactccatctccatgcccatgtgggacgaggaggaggatgaggaggccaagaagcagacccccaagcagaggctcctgggctggatccagaacaagctgccgcagctgcccatcaccaacttcagccgggactggcagagcggccgggccctgggcgccctggtggacagctgtgccccgggcctgtgtcctgactgggactcttgggacgccagcaagcccgttaccaatgcgcgagaggccatgcagcaggcggatgactggctgggcatcccccaggtgatcacccccgaggagattgtggaccccaacgtggacgagcactctgtcatgacctacctgtcccagttccccaaggccaagctgaagccaggggctcccttgcggcccaaactgaacccgaagaaagcccgtgcctacgggccaggcatcgagcccacaggcaacatggtgaagaagcgggcagagttcactgtggagaccagaagtgctggccagggagaggtgctggtgtacgtggaggacccggccggacaccaggaggaggcaaaagtgaccgccaataacgacaagaaccgcaccttctccgtctggtacgtccccgaggtgacggggactcataaggttactgtgctctttgctggccagcacatcgccaagagccccttcgaggtgtacgtggataagtcacagggtgacgccagcaaagtgacagcccaaggtcccggcctggagcccagtggcaacatcgccaacaagaccacctactttgagatctttacggcaggagctggcacgggcgaggtcgaggttgtgatccaggaccccatgggacagaagggcacggtagagcctcagctggaggcccggggcgacagcacataccgctgcagctaccagcccaccatggagggcgtccacaccgtgcacgtcacgtttgccggcgtgcccatccctcgcagcccctacactgtcactgttggccaagcctgtaacccgagtgcctgccgggcggttggccggggcctccagcccaagggtgtgcgggtgaaggagacagctgacttcaaggtgtacacaaagggcgctggcagtggggagctgaaggtcaccgtgaagggccccaagggagaggagcgcgtgaagcagaaggacctgggggatggcgtgtatggcttcgagtattaccccatggtccctggaacctatatcgtcaccatcacgtggggtggtcagaacatcgggcgcagtcccttcgaagtgaaggtgggcaccgagtgtggcaatcagaaggtacgggcctggggccctgggctggagggcggcgtcgttggcaagtcagcagactttgtggtggaggctatcggggacgacgtgggcacgctgggcttctcggtggaagggccatcgcaggctaagatcgaatgtgacgacaagggcgacggctcctgtgatgtgcgctactggccgcaggaggctggcgagtatgccgttcacgtgctgtgcaacagcgaagacatccgcctcagccccttcatggctgacatccgtgacgcgccccaggacttccacccagacagggtgaaggcacgtgggcctggattggagaagacaggtgtggccgtcaacaagccagcagagttcacagtggatgccaagcacggtggcaaggccccacttcgggtccaagtccaggacaatgaaggctgccctgtggaggcgttggtcaaggacaacggcaatggcacttacagctgctcctacgtgcccaggaagccggtgaagcacacagccatggtgtcctggggaggcgtcagcatccccaacagccccttcagggtgaatgtgggagctggcagccaccccaacaaggtcaaagtatacggccccggagtagccaagacagggctcaaggcccacgagcccacctacttcactgtggactgcgccgaggctggccagggggacgtcagcatcggcatcaagtgtgcccctggagtggtaggccccgccgaagctgacatcgacttcgacatcatccgcaatgacaatgacaccttcacggtcaagtacacgccccggggggctggcagctacaccattatggtcctctttgctgaccaggccacgcccaccagccccatccgagtcaaggtggagccctctcatgacgccagtaaggtgaaggccgagggccctggcctcagtcgcactggtgtcgagcttggcaagcccacccacttcacagtaaatgccaaagctgctggcaaaggcaagctggacgtccagttctcaggactcaccaagggggatgcagtgcgagatgtggacatcatcgaccaccatgacaacacctacacagtcaagtacacgcctgtccagcagggtccagtaggcgtcaatgtcacttatggaggggatcccatccctaagagccctttctcagtggcagtatctccaagcctggacctcagcaagatcaaggtgtctggcctgggagagaaggtggacgttggcaaagaccaggagttcacagtcaaatcaaagggtgctggtggtcaaggcaaagtggcatccaagattgtgggcccctcgggtgcagcggtgccctgcaaggtggagccaggcctgggggctgacaacagtgtggtgcgcttcctgccccgtgaggaagggccctatgaggtggaggtgacctatgacggcgtgcccgtgcctggcagcccctttcctctggaagctgtggcccccaccaagcctagcaaggtgaaggcgtttgggccggggctgcagggaggcagtgcgggctcccccgcccgcttcaccatcgacaccaagggcgccggcacaggtggcctgggcctgacggtggagggcccctgtgaggcgcagctcgagtgcttggacaatggggatggcacatgttccgtgtcctacgtgcccaccgagcccggggactacaacatcaacatcctcttcgctgacacccacatccctggctccccattcaaggcccacgtggttccctgctttgacgcatccaaagtcaagtgctcaggccccgggctggagcgggccaccgctggggaggtgggccaattccaagtggactgctcgagcgcgggcagcgcggagctgaccattgagatctgctcggaggcggggcttccggccgaggtgtacatccaggaccacggtgatggcacgcacaccattacctacattcccctctgccccggggcctacaccgtcaccatcaagtacggcggccagcccgtgcccaacttccccagcaagctgcaggtggaacctgcggtggacacttccggtgtccagtgctatgggcctggtattgagggccagggtgtcttccgtgaggccaccactgagttcagtgtggacgcccgggctctgacacagaccggagggccgcacgtcaaggcccgtgtggccaacccctcaggcaacctgacggagacctacgttcaggaccgtggcgatggcatgtacaaagtggagtacacgccttacgaggagggactgcactccgtggacgtgacctatgacggcagtcccgtgcccagcagccccttccaggtgcccgtgaccgagggctgcgacccctcccgggtgcgtgtccacgggccaggcatccaaagtggcaccaccaacaagcccaacaagttcactgtggagaccaggggagctggcacgggcggcctgggcctggctgtagagggcccctccgaggccaagatgtcctgcatggataacaaggacggcagctgctcggtcgagtacatcccttatgaggctggcacctacagcctcaacgtcacctatggtggccatcaagtgccaggcagtcctttcaaggtccctgtgcatgatgtgacagatgcgtccaaggtcaagtgctctgggcccggcctgagcccaggcatggttcgtgccaacctccctcagtccttccaggtggacacaagcaaggctggtgtggccccattgcaggtcaaagtgcaagggcccaaaggcctggtggagccagtggacgtggtagacaacgctgatggcacccagaccgtcaattatgtgcccagccgagaagggccctacagcatctcagtactgtatggagatgaagaggtaccccggagccccttcaaggtcaaggtgctgcctactcatgatgccagcaaggtgaaggccagtggccccgggctcaacaccactggcgtgcctgccagcctgcccgtggagttcaccatcgatgcaaaggacgccggggagggcctgctggctgtccagatcacggatcccgaaggcaagccgaagaagacacacatccaagacaaccatgacggcacgtatacagtggcctacgtgccagacgtgacaggtcgctacaccatcctcatcaagtacggtggtgacgagatccccttctccccgtaccgcgtgcgtgccgtgcccaccggggacgccagcaagtgcactgtcacagtgtcaatcggaggtcacgggctaggtgctggcatcggccccaccattcagattggggaggagacggtgatcactgtggacactaaggcggcaggcaaaggcaaagtgacgtgcaccgtgtgcacgcctgatggctcagaggtggatgtggacgtggtggagaatgaggacggcactttcgacatcttctacacggccccccagccgggcaaatacgtcatctgtgtgcgctttggtggcgagcacgtgcccaacagccccttccaagtgacggctctggctggggaccagccctcggtgcagccccctctacggtctcagcagctggccccacagtacacctacgcccagggcggccagcagacttgggccccggagaggcccctggtgggtgtcaatgggctggatgtgaccagcctgaggccctttgaccttgtcatccccttcaccatcaagaagggcgagatcacaggggaggttcggatgccctcaggcaaggtggcgcagcccaccatcactgacaacaaagacggcaccgtgaccgtgcggtatgcacccagcgaggctggcctgcacgagatggacatccgctatgacaacatgcacatcccaggaagccccttgcagttctatgtggattacgtcaactgtggccatgtcactgcctatgggcctggcctcacccatggagtagtgaacaagcctgccaccttcaccgtcaacaccaaggatgcaggagaggggggcctgtctctggccattgagggcccgtccaaagcagaaatcagctgcactgacaaccaggatgggacatgcagcgtgtcctacctgcctgtgctgccgggggactacagcattctagtcaagtacaatgaacagcacgtcccaggcagccccttcactgctcgggtcacaggtgacgactccatgcgtatgtcccacctaaaggtcggctctgctgccgacatccccatcaacatctcagagacggatctcagcctgctgacggccactgtggtcccgccctcgggccgggaggagccctgtttgctgaagcggctgcgtaatggccacgtggggatttcattcgtgcccaaggagacgggggagcacctggtgcatgtgaagaaaaatggccagcacgtggccagcagccccatcccggtggtgatcagccagtcggaaattggggatgccagtcgtgttcgggtctctggtcagggccttcacgaaggccacacctttgagcctgcagagtttatcattgatacccgcgatgcaggctatggtgggctcagcctgtccattgagggccccagcaaggtggacatcaacacagaggacctggaggacgggacgtgcagggtcacctactgccccacagagccaggcaactacatcatcaacatcaagtttgccgaccagcacgtgcctggcagccccttctctgtgaaggtgacaggcgagggccgggtgaaagagagcatcacccgcaggcgtcgggctccttcagtggccaacgttggtagtcattgtgacctcagcctgaaaatccctgaaattagcatccaggatatgacagcccaggtgaccagcccatcgggcaagacccatgaggccgagatcgtggaaggggagaaccacacctactgcatccgctttgttcccgctgagatgggcacacacacagtcagcgtgaagtacaagggccagcacgtgcctgggagccccttccagttcaccgtggggcccctaggggaagggggagcccacaaggtccgagctgggggccctggcctggagagagctgaagctggagtgccagccgaattcagtatctggacccgggaagctggtgctggaggcctggccattgctgtcgagggccccagcaaggctgagatctcttttgaggaccgcaaggacggctcctgtggtgtggcttatgtggtccaggagccaggtgactacgaagtctcagtcaagttcaacgaggaacacattcccgacagccccttcgtggtgcctgtggcttctccgtctggcgacgcccgccgcctcactgtttctagccttcgggagtcagggctaaaggtcaaccagccagcctcttttgcagtcagcctgaacggggccaagggggcgatcgatgccaaggtgcacagcccctcaggagccctggaggagtgctatgtcacagaaattgaccaagataagtatgctgtgcgcttcatccctcgggagaatggcgtttacctgattgacgtcaagttcaacggcacccacatccctggaagccccttcaagatccgagttggggagcctgggcatggaggggacccaggcttggtgtctgcttacggagcaggtctggaaggcggtgtcacagggaacccagctgagttcgtcgtgaacacgagcaatgcgggagctggtgccctgtcggtgaccattgacggcccctccaaggtgaagatggattgccaggagtgccctgagggctaccgcgtcacctatacccccatggcacctggcagctacctcatctccatcaagtacggcggcccctaccacattgggggcagccccttcaaggccaaagtcacaggcccccgtctcgtcagcaaccacagcctccacgagacatcatcagtgtttgtagactctctgaccaaggccacctgtgccccccagcatggggccccgggtcctgggcctgctgacgccagcaaggtggtggccaagggcctggggctgagcaaggcctacgtaggccagaagagcagcttcacagtagactgcagcaaagcaggcaacaacatgctgctggtgggggttcatggcccaaggaccccctgcgaggagatcctggtgaagcacgtgggcagccggctctacagcgtgtcctacctgctcaaggacaagggggagtacacactggtggtcaaatggggggacgagcacatcccaggcagcccctaccgcgttgtggtgccctga

SEQ ID NO: 5 - protein, 2633 aa, Homo sapiens
  filamin-B isoform 1 modified
MPVTEKDLAEDAPWKKIQQNTFTRWCNEHLKCVNKRIGNLQTDLSDGLRLIALLEVLSQKRMYRKYHQRPTFRQMQLENVSVALEFLDRESIKLVSIDSKAIVDGNLKLILGLVWTLILHYSISMPVWEDEGDDDAKKQTPKQRLLGWIQNKIPYLPITNFNQNWQDGKALGALVDSCAPGLCPDWESWDPQKPVDNAREAMQQADDWLGVPQVITPEEIIHPDVDEHSVMTYLSQFPKAKLKPGAPLKPKLNPKKARAYGRGIEPTGNMVKQPAKFTVDTISAGQGDVMVFVEDPEGNKEEAQVTPDSDKNKTYSVEYLPKVTGLHKVTVLFAGQHISKSPFEVSVDKAQGDASKVTAKGPGLEAVGNIANKPTYFDIYTAGAGVGDIGVEVEDPQGKNTVELLVEDKGNQVYRCVYKPMQPGPHVVKIFFAGDTIPKSPFVVQVGEACNPNACRASGRGLQPKGVRIRETTDFKVDTKAAGSGELGVTMKGPKGLEELVKQKDFLDGVYAFEYYPSTPGRYSIAITWGGHHIPKSPFEVQVGPEAGMQKVRAWGPGLHGGIVGRSADFVVESIGSEVGSLGFAIEGPSQAKIEYNDQNDGSCDVKYWPKEPGEYAVHIMCDDEDIKDSPYMAFIHPATGGYNPDLVRAYGPGLEKSGCIVNNLAEFTVDPKDAGKAPLKIFAQDGEGQRIDIQMKNRMDGTYACSYTPVKAIKHTIAVVWGGVNIPHSPYRVNIGQGSHPQKVKVFGPGVERSGLKANEPTHFTVDCTEAGEGDVSVGIKCDARVLSEDEEDVDFDIIHNANDTFTVKYVPPAAGRYTIKVLFASQEIPASPFRVKVDPSHDASKVKAEGPGLSKAGVENGKPTHFTVYTKGAGKAPLNVQFNSPLPGDAVKDLDIIDNYDYSHTVKYTPTQQGNMQVLVTYGGDPIPKSPFTVGVAAPLDLSKIKLNGLENRVEVGKDQEFTVDTRGAGGQGKLDVTILSPSRKVVPCLVTPVTGRENSTAKFIPREEGLYAVDVTYDGHPVPGSPYTVEASLPPDPSKVKAHGPGLEGGLVGKPAEFTIDTKGAGTGGLGLTVEGPCEAKIECSDNGDGTCSVSYLPTKPGEYFVNILFEEVHIPGSPFKADIEMPFDPSKVVASGPGLEHGKVGEAGLLSVDCSEAGPGALGLEAVSDSGTKAEVSIQNNKDGTYAVTYVPLTAGMYTLTMKYGGELVPHFPARVKVEPAVDTSRIKVFGPGIEGKDVFREATTDFTVDSRPLTQVGGDHIKAHIANPSGASTECFVTDNADGTYQVEYTPFEKGLHVVEVTYDDVPIPNSPFKVAVTEGCQPSRVQAQGPGLKEAFTNKPNVFTVVTRGAGIGGLGITVEGPSESKINCRDNKDGSCSAEYIPFAPGDYDVNITYGGAHIPGSPFRVPVKDVVDPSKVKIAGPGLGSGVRARVLQSFTVDSSKAGLAPLEVRVLGPRADDTDSQSWRSPLKALSEFFKGDPKGDFNKTGLVEPVNVVDNGDGTHTVTYTPSQEGPYMVSVKYADEEIPRSPFKVKVLPTYDASKVTASGPGLSSYGVPASLPVDFAIDARDAGEGLLAVQITDQEGKPKRAIVHDNKDGTYAVTYIPDKTGRYMIGVTYGGDDIPLSPYRIRATQTGDASKCLATGPGIASTVKTGEEVGFVVDAKTAGKGKVTCTVLTPDGTEAEADVIENEDGTYDIFYTAAKPGTYVIYVRFGGVDIPNSPFTVMATDGEVTAVEEAPVNACPPGFRPWVTEEAYVPVSDMNGLGFKPFDLVIPFAVRKGEITGEVHMPSGKTATPEIVDNKDGTVTVRYAPTEVGLHEMHIKYMGSHIPESPLQFYVNYPNSGSVSAYGPGLVYGVANKTATFTIVTEDAGEGGLDLAIEGPSKAEISCIDNKDGTCTVTYLPTLPGDYSILVKYNDKHIPGSPFTAKITDDSRRCSQVKLGSAADFLLDISETDLSSLTASIKAPSGRDEPCLLKRLPNNHIGISFIPREVGEHLVSIKKNGNHVANSPVSIMVVQSEIGDARRAKVYGRGLSEGRTFEMSDFIVDTRDAGYGGISLAVEGPSKVDIQTEDLEDGTCKVSYFPTVPGVYIVSTKFADEHVPGSPFTVKISGEGRVKESITRTSRAPSVATVGSICDLNLKIPEINSSDMSAHVTSPSGRVTEAEIVPMGKNSHCVRFVPQEMGVHTVSVKYRGQHVTGSPFQFTVGPLGEGGAHKVRAGGPGLERGEAGVPAEFSIWTREAGAGGLSIAVEGPSKAEITFDDHKNGSCGVSYIAQEPGNYEVSIKFNDEHIPESPYLVPVIAPSDDARRLTVMSLRESGLKVNQPASFAIRLNGAKGKIDAKVHSPSGAVEECHVSELEPDKYAVRFIPHENGVHTIDVKFNGSHVVGSPFKVRVGEPGQAGNPALVSAYGTGLEGGTTGIQSEFFINTTRAGPGTLSVTIEGPSKVKMDCQETPEGYKVMYTPMAPGNYLISVKYGGPNHIVGSPFKAKVTGQRLVSPGSANETSSILVESVTRSSTETCYSAIPKASSDASKVTSKGAGLSKAFVGQKSSFLVDCSKAGSNMLLIGVHGPTTPCEEVSMKHVGNQQYNVTYVVKERGDYVLAVKWGEEHIPGSPFHVTVP

SEQ ID NO: 6 - DNA, 7902 bp, Homo sapiens
  Filamin B isoform 1 modified cDNA sequence
atgccggtaaccgagaaggatctagctgaggacgcgccttggaagaagatccagcagaacacgttcacacgctggtgcaacgagcacctcaagtgcgtgaacaaacgcatcggcaacctgcagaccgacctgagcgacgggctgcggctcatcgcgctgctcgaggtgctcagccagaagcgcatgtaccgcaagtaccatcagcggcccacctttcgccagatgcagctcgagaatgtgtccgtggcgctcgagttcctggaccgtgagagcatcaagctcgtgtccatcgatagcaaagccattgtggatgggaacctgaagctcatcttgggtctggtgtggacgctgatcctccactactccatctccatgcccgtgtgggaggatgaaggggatgatgatgccaagaagcagacgccaaagcagaggctgctggggtggattcagaacaagatcccctacttgcccatcaccaactttaaccagaactggcaagacggcaaagccctgggagccctggtagacagctgtgctccaggtctgtgcccagactgggaatcctgggacccgcagaagcctgtggataatgcacgagaagccatgcagcaggcagatgactggctgggtgtcccacaggtcatcactcctgaagaaatcattcacccggatgtggacgagcactcagttatgacttacctgtcccagttccccaaagccaagctcaagccgggggctcctctcaaacccaaactcaacccgaagaaagccagggcctatggcagaggaatcgagcccactggaaacatggtgaagcagccagccaagttcactgtggacaccatcagcgccgggcaaggagacgtgatggtgtttgttgaggacccagaagggaacaaagaggaggcacaagtgacccctgacagtgacaagaacaagacatactctgtggagtatctgcccaaggtcaccgggctacacaaagtcacagtcctctttgcaggacagcacatctccaagagcccatttgaagtgagtgttgacaaggcccagggagatgccagtaaagtcactgcaaaaggtccagggttggaagctgtagggaacatcgccaataagcccacctactttgacatctatacggcaggagctggtgtgggtgacattggtgtggaggtggaagatccccaggggaagaacaccgtggagttgctcgtggaagacaaaggaaaccaggtgtatcgatgtgtgtacaaacccatgcagcctggccctcacgtggtcaagatcttctttgctggggacactattcctaagagtcccttcgttgtgcaggttggggaagcctgcaatccaaatgcctgccgggccagtggccgaggcctacaacccaaaggcgtccgtatccgggagaccacagatttcaaggttgacaccaaagctgcaggaagtggggagctcggtgtaaccatgaagggtcctaagggtctggaggagctggtgaagcagaaagactttctggatggggtctacgcattcgagtattaccccagcaccccggggagatacagcattgccatcacatgggggggacaccacattccaaagagcccctttgaagttcaagttggccctgaagcgggtatgcagaaagtccgtgcttggggccctgggctccatggtgggattgtcgggcggtcagcggacttcgtggtagaatccattggctctgaagtggggtctctggggtttgccattgaaggcccctctcaggcaaagattgagtacaacgaccagaatgatggatcgtgtgatgtcaaatactggcccaaggagcctggcgaatatgctgttcacatcatgtgtgacgacgaagacatcaaggacagcccgtacatggccttcatccacccagccacgggaggctacaaccctgatctggttcgagcatacgggccaggtttggagaaatctggatgcattgtcaacaacctggccgagttcactgtggatcctaaggatgctggaaaagctcccttaaagatatttgctcaggatggggaaggccaacgcattgacatccagatgaagaaccggatggacggcacatatgcatgctcatacaccccggtgaaggccatcaagcacaccattgctgtggtctggggaggcgtgaacatcccgcacagcccctacagggtcaacatcgggcaaggtagccatcctcagaaggtcaaagtgtttgggccaggtgtggagagaagtggtctgaaggcaaatgaacctacacacttcacggtggactgtactgaggctggggaaggtgatgtcagtgttggcattaagtgtgatgcccgggtgttaagtgaagatgaggaagacgtggattttgacattattcacaatgccaatgatacgttcacagtcaaatatgtgcctcctgctgctgggcgatacactatcaaagttctctttgcatctcaggaaatccccgccagccctttcagagtcaaagttgacccttcccacgatgccagcaaagtgaaggcagaaggcccagggctcagcaaagcaggtgtggaaaatgggaaaccgacccacttcactgtctacaccaagggggctgggaaagccccgctcaacgtgcagttcaacagccctcttcctggcgatgcagtgaaggatttggatatcatcgataattatgactactctcacacggttaaatatacacccacccaacagggcaacatgcaggttctggtgacttacggtggcgatcccatccctaaaagccctttcactgtgggtgttgctgcaccgctggatctgagcaagataaaactcaatgggctggaaaacagggtggaagttgggaaggatcaggagttcaccgttgataccaggggggcaggaggccaggggaagctggacgtgacaatcctcagcccctctcggaaggtcgtgccatgcctagtgacacctgtgacaggccgggagaacagcacggccaagttcatccctcgggaggaggggctgtatgctgtagacgtgacctacgatggacaccctgtgcccgggagcccctacacagtggaggcctcgctgccaccagatcccagcaaggtgaaggcccacggtcccggcctcgaaggtggtctcgtgggcaagcctgccgagttcaccatcgataccaaaggagctggtactggaggtctgggcttaacggtggaaggtccgtgcgaggccaaaatcgagtgctccgacaatggtgatgggacctgctccgtctcttaccttcccacaaaacccggggagtacttcgtcaacatcctctttgaagaagtccacatacctgggtctcccttcaaagctgacattgaaatgccctttgacccctctaaagtcgtggcatcggggccaggtctcgagcacgggaaggtgggtgaagctggcctccttagcgtcgactgctcggaagcgggaccgggggccctgggcctggaagctgtctcggactcgggaacaaaagccgaagtcagtattcagaacaacaaagatggcacctacgcggtgacctacgtgcccctgacggccggcatgtacacgttgaccatgaagtatggtggcgaactcgtgccacacttccccgcccgggtcaaggtggagcccgccgtggacaccagcaggatcaaagtctttggaccaggaatagaagggaaagatgtgttccgggaagctaccaccgactttacagttgactctcggccgctgacccaggttgggggtgaccacatcaaggcccacattgccaacccctcaggggcctccaccgagtgctttgtcacagacaatgcggatgggacctaccaggtggaatacacaccctttgagaaaggtctccatgtagtggaggtgacatatgatgacgtgcctatcccaaacagtcccttcaaggtggctgtcactgaaggctgccagccatctagggtgcaagcccaaggacctggattgaaagaggcctttaccaacaagcccaatgtcttcaccgtggttaccagaggcgcaggaattggtgggcttggcataactgttgagggaccatcagagtcgaagataaattgcagagacaacaaggatggcagctgcagtgctgagtacattcctttcgcaccgggggattacgatgttaatatcacatatggaggagcccacatccccggcagccccttcagggttcctgtgaaggatgttgtggaccccagcaaggtcaagattgccggccccgggctgggctcaggcgtccgagcccgtgtcctgcagtccttcacggtggacagcagcaaggctggcctggctccgctggaagtgagggttctgggcccacgagctgacgacacggattcccagtcatggcgcagccccttgaaagccctttcagagttctttaaaggtgacccgaagggtgactttaataagacaggcttggtggagccagtgaacgtggtggacaatggagatggcacacacacagtaacctacaccccatctcaggagggaccttacatggtctcagttaaatatgctgatgaagagattcctcgcagtcccttcaaggtcaaggtccttcccacatatgatgccagcaaagtgactgccagtggccccggccttagttcctatggtgtgcctgccagtctacctgtggactttgcaattgatgcccgagatgccggggaaggcctgcttgctgttcaaataacggaccaagaaggaaaacccaaaagagccattgtccatgacaataaagatggcacgtatgctgtcacctacatccccgacaagactgggcgctatatgattggagtcacctacgggggtgacgacatcccactttctccttatcgcatccgagccacacagacgggtgatgccagcaagtgcctggccacgggtcctggaatcgcctccactgtgaaaactggcgaagaagtaggctttgtggttgatgccaagactgccgggaagggtaaagtgacctgcacggttctgaccccagatggcactgaggccgaggccgatgtcattgagaatgaagatggaacctatgacatcttctacacagctgccaagccgggcacatatgtgatctatgtgcgcttcggtggtgttgatattcctaacagccccttcactgtcatggccacagatggggaagtcacagccgtggaggaggcaccggtaaatgcatgtccccctggattcaggccctgggtgaccgaagaggcctatgtcccagtgagtgacatgaacggcctgggatttaagccttttgacctggtcattccgtttgctgtcaggaaaggagaaatcactggagaggtccacatgccttctgggaagacagccacacctgagattgtggacaacaaggacggcacggtcactgttagatatgcccccactgaggtcgggctccatgagatgcacatcaaatacatgggcagccacatccctgagagcccactccagttctacgtgaactaccccaacagtggaagtgtttctgcatacggtccaggcctcgtgtatggagtggccaacaaaactgccaccttcaccatcgtcacagaggatgcaggagaaggtggtctggacttggctattgagggcccctcaaaagcagaaatcagctgcattgacaataaagatgggacatgcacagtgacctacctgccgactctgccaggcgactacagcattctggtcaagtacaatgacaagcacatccctggcagccccttcacagccaagatcacagatgacagcaggcggtgctcccaggtgaagttgggctcagccgctgacttcctgctcgacatcagtgagactgacctcagcagcctgacggccagcattaaggccccatctggccgagacgagccctgtctcctgaagaggctgcccaacaaccacattggcatctccttcatcccccgggaagtgggcgaacatctggtcagcatcaagaaaaatggcaaccatgtggccaacagccccgtgtctatcatggtggtccagtcggagattggtgacgcccgccgagccaaagtctatggccgcggcctgtcagaaggccggactttcgagatgtctgacttcatcgtggacacaagggatgcaggttatggtggcatatccttggcggtggaaggccccagcaaagtggacatccagacggaggacctggaagatggcacctgcaaagtctcctacttccctaccgtgcctggggtttatatcgtctccaccaaattcgctgacgagcacgtgcctgggagcccatttaccgtgaagatcagtggggagggaagagtcaaagagagcatcacccgcaccagtcgggccccgtccgtggccactgtcgggagcatttgtgacctgaacctgaaaatcccagaaatcaacagcagtgatatgtcggcccacgtcaccagcccctctggccgtgtgactgaggcagagattgtgcccatggggaagaactcacactgcgtccggtttgtgccccaggagatgggcgtgcacacggtcagcgtcaagtaccgtgggcagcacgtcaccggcagccccttccagttcaccgtggggccacttggtgaaggaggcgcccacaaggtgcgggcaggaggccctggcctggagagaggagaagcgggagtcccagctgagttcagcatttggacccgggaagcaggcgctggaggcctctccatcgctgttgagggccccagtaaggccgagattacattcgatgaccataaaaatgggtcgtgcggtgtatcttatattgcccaagagcctggtaactacgaggtgtccatcaagttcaatgatgagcacatcccggaaagcccctacctggtgccggtcatcgcaccctccgacgacgcccgccgcctcactgttatgagccttcgggaatcgggattaaaagttaaccagccagcatcctttgctataaggttgaatggcgcaaaaggcaagattgatgcaaaggtgcacagcccctctggagccgtggaggagtgccacgtgtctgagctggagccagataagtatgctgttcgcttcatccctcatgagaatggtgtccacaccatcgatgtcaagttcaatgggagccacgtggttggaagccccttcaaagtgcgcgttggggagcctggacaagcggggaaccctgccctggtgtccgcctatggcacgggactcgaagggggcaccacaggtatccagtcggaattctttattaacaccacccgagcaggtccagggacattatccgtcaccatcgaaggcccatccaaggttaaaatggattgccaggaaacacctgaagggtacaaagtcatgtacacccccatggctcctggtaactacctgatcagcgtcaaatacggtgggcccaaccacatcgtgggcagtcccttcaaggccaaggtgacaggccagcgtctagttagccctggctcagccaacgagacctcatccatcctggtggagtcagtgaccaggtcgtctacagagacctgctatagcgccattcccaaggcatcctcggacgccagcaaggtgacctctaagggggcagggctctcaaaggcctttgtgggccagaagagttccttcctggtggactgcagcaaagctggctccaacatgctgctgatcggggtccatgggcccaccaccccctgcgaggaggtctccatgaagcatgtaggcaaccagcaatacaacgtcacatacgtcgtcaaggagaggggcgattatgtgctggctgtgaagtggggggaggaacacatccctggcagcccttttcatgtcacagtgccttaa

SEQ ID NO: 7 - RNA, 8533 nt, Homo sapiens
  filamin A isoform 1 mRNA
auucgcguggaggcgcgucgcgcgcagcggacgccgacagaauccccgaggcgccuggcgcgggcgcgggcgcgaaggcgauccgggcgccaccccgcggucaucggucaccggucgcucucaggaacagcagcgcaaccucugcucccugccucgccucccgcgcgccuaggugccugcgacuuuaauuaaagggccguccccucgccgaggcugcagcaccgcccccccggcuucucgcgccucaaaaugaguagcucccacucucgggcgggccagagcgcagcaggcgcggcuccgggcggcggcgucgacacgcgggacgccgagaugccggccaccgagaaggaccuggcggaggacgcgccguggaagaagauccagcagaacacuuucacgcgcuggugcaacgagcaccugaagugcgugagcaagcgcaucgccaaccugcagacggaccugagcgacgggcugcggcuuaucgcgcuguuggaggugcucagccagaagaagaugcaccgcaagcacaaccagcggcccacuuuccgccaaaugcagcuugagaacgugucgguggcgcucgaguuccuggaccgcgagagcaucaaacugguguccaucgacagcaaggccaucguggacgggaaccugaagcugauccugggccucaucuggacccugauccugcacuacuccaucuccaugcccaugugggacgaggaggaggaugaggaggccaagaagcagacccccaagcagaggcuccugggcuggauccagaacaagcugccgcagcugcccaucaccaacuucagccgggacuggcagagcggccgggcccugggcgcccugguggacagcugugccccgggccuguguccugacugggacucuugggacgccagcaagcccguuaccaaugcgcgagaggccaugcagcaggcggaugacuggcugggcaucccccaggugaucacccccgaggagauuguggaccccaacguggacgagcacucugucaugaccuaccugucccaguuccccaaggccaagcugaagccaggggcucccuugcggcccaaacugaacccgaagaaagcccgugccuacgggccaggcaucgagcccacaggcaacauggugaagaagcgggcagaguucacuguggagaccagaagugcuggccagggagaggugcugguguacguggaggacccggccggacaccaggaggaggcaaaagugaccgccaauaacgacaagaaccgcaccuucuccgucugguacguccccgaggugacggggacucauaagguuacugugcucuuugcuggccagcacaucgccaagagccccuucgagguguacguggauaagucacagggugacgccagcaaagugacagcccaaggucccggccuggagcccaguggcaacaucgccaacaagaccaccuacuuugagaucuuuacggcaggagcuggcacgggcgaggucgagguugugauccaggaccccaugggacagaagggcacgguagagccucagcuggaggcccggggcgacagcacauaccgcugcagcuaccagcccaccauggagggcguccacaccgugcacgucacguuugccggcgugcccaucccucgcagccccuacacugucacuguuggccaagccuguaacccgagugccugccgggcgguuggccggggccuccagcccaagggugugcgggugaaggagacagcugacuucaagguguacacaaagggcgcuggcaguggggagcugaaggucaccgugaagggccccaagggagaggagcgcgugaagcagaaggaccugggggauggcguguauggcuucgaguauuaccccauggucccuggaaccuauaucgucaccaucacgugggguggucagaacaucgggcgcagucccuucgaagugaaggugggcaccgaguguggcaaucagaagguacgggccuggggcccugggcuggagggcggcgucguuggcaagucagcagacuuugugguggaggcuaucggggacgacgugggcacgcugggcuucucgguggaagggccaucgcaggcuaagaucgaaugugacgacaagggcgacggcuccugugaugugcgcuacuggccgcaggaggcuggcgaguaugccguucacgugcugugcaacagcgaagacauccgccucagccccuucauggcugacauccgugacgcgccccaggacuuccacccagacagggugaaggcacgugggccuggauuggagaagacagguguggccgucaacaagccagcagaguucacaguggaugccaagcacgguggcaaggccccacuucggguccaaguccaggacaaugaaggcugcccuguggaggcguuggucaaggacaacggcaauggcacuuacagcugcuccuacgugcccaggaagccggugaagcacacagccaugguguccuggggaggcgucagcauccccaacagccccuucagggugaaugugggagcuggcagccaccccaacaaggucaaaguauacggccccggaguagccaagacagggcucaaggcccacgagcccaccuacuucacuguggacugcgccgaggcuggccagggggacgucagcaucggcaucaagugugccccuggagugguaggccccgccgaagcugacaucgacuucgacaucauccgcaaugacaaugacaccuucacggucaaguacacgccccggggggcuggcagcuacaccauuaugguccucuuugcugaccaggccacgcccaccagccccauccgagucaagguggagcccucucaugacgccaguaaggugaaggccgagggcccuggccucagucgcacuggugucgagcuuggcaagcccacccacuucacaguaaaugccaaagcugcuggcaaaggcaagcuggacguccaguucucaggacucaccaagggggaugcagugcgagauguggacaucaucgaccaccaugacaacaccuacacagucaaguacacgccuguccagcaggguccaguaggcgucaaugucacuuauggaggggaucccaucccuaagagcccuuucucaguggcaguaucuccaagccuggaccucagcaagaucaaggugucuggccugggagagaagguggacguuggcaaagaccaggaguucacagucaaaucaaagggugcugguggucaaggcaaaguggcauccaagauugugggccccucgggugcagcggugcccugcaagguggagccaggccugggggcugacaacaguguggugcgcuuccugccccgugaggaagggcccuaugagguggaggugaccuaugacggcgugcccgugccuggcagccccuuuccucuggaagcuguggcccccaccaagccuagcaaggugaaggcguuugggccggggcugcagggaggcagugcgggcucccccgcccgcuucaccaucgacaccaagggcgccggcacagguggccugggccugacgguggagggccccugugaggcgcagcucgagugcuuggacaauggggauggcacauguuccguguccuacgugcccaccgagcccggggacuacaacaucaacauccucuucgcugacacccacaucccuggcuccccauucaaggcccacgugguucccugcuuugacgcauccaaagucaagugcucaggccccgggcuggagcgggccaccgcuggggaggugggccaauuccaaguggacugcucgagcgcgggcagcgcggagcugaccauugagaucugcucggaggcggggcuuccggccgagguguacauccaggaccacggugauggcacgcacaccauuaccuacauuccccucugccccggggccuacaccgucaccaucaaguacggcggccagcccgugcccaacuuccccagcaagcugcagguggaaccugcgguggacacuuccgguguccagugcuaugggccugguauugagggccagggugucuuccgugaggccaccacugaguucaguguggacgcccgggcucugacacagaccggagggccgcacgucaaggcccguguggccaaccccucaggcaaccugacggagaccuacguucaggaccguggcgauggcauguacaaaguggaguacacgccuuacgaggagggacugcacuccguggacgugaccuaugacggcagucccgugcccagcagccccuuccaggugcccgugaccgagggcugcgaccccucccgggugcguguccacgggccaggcauccaaaguggcaccaccaacaagcccaacaaguucacuguggagaccaggggagcuggcacgggcggccugggccuggcuguagagggccccuccgaggccaagauguccugcauggauaacaaggacggcagcugcucggucgaguacaucccuuaugaggcuggcaccuacagccucaacgucaccuaugguggccaucaagugccaggcaguccuuucaaggucccugugcaugaugugacagaugcguccaaggucaagugcucugggcccggccugagcccaggcaugguucgugccaaccucccucaguccuuccagguggacacaagcaaggcugguguggccccauugcaggucaaagugcaagggcccaaaggccugguggagccaguggacgugguagacaacgcugauggcacccagaccgucaauuaugugcccagccgagaagggcccuacagcaucucaguacuguauggagaugaagagguaccccggagccccuucaaggucaaggugcugccuacucaugaugccagcaaggugaaggccaguggccccgggcucaacaccacuggcgugccugccagccugcccguggaguucaccaucgaugcaaaggacgccggggagggccugcuggcuguccagaucacggaucccgaaggcaagccgaagaagacacacauccaagacaaccaugacggcacguauacaguggccuacgugccagacgugacaggucgcuacaccauccucaucaaguacgguggugacgagauccccuucuccccguaccgcgugcgugccgugcccaccggggacgccagcaagugcacugucacaggugcuggcaucggccccaccauucagauuggggaggagacggugaucacuguggacacuaaggcggcaggcaaaggcaaagugacgugcaccgugugcacgccugauggcucagagguggauguggacgugguggagaaugaggacggcacuuucgacaucuucuacacggccccccagccgggcaaauacgucaucugugugcgcuuugguggcgagcacgugcccaacagccccuuccaagugacggcucuggcuggggaccagcccucggugcagcccccucuacggucucagcagcuggccccacaguacaccuacgcccagggcggccagcagacuugggccccggagaggccccuggugggugucaaugggcuggaugugaccagccugaggcccuuugaccuugucauccccuucaccaucaagaagggcgagaucacaggggagguucggaugcccucaggcaagguggcgcagcccaccaucacugacaacaaagacggcaccgugaccgugcgguaugcacccagcgaggcuggccugcacgagauggacauccgcuaugacaacaugcacaucccaggaagccccuugcaguucuauguggauuacgucaacuguggccaugucacugccuaugggccuggccucacccauggaguagugaacaagccugccaccuucaccgucaacaccaaggaugcaggagaggggggccugucucuggccauugagggcccguccaaagcagaaaucagcugcacugacaaccaggaugggacaugcagcguguccuaccugccugugcugccgggggacuacagcauucuagucaaguacaaugaacagcacgucccaggcagccccuucacugcucgggucacaggugacgacuccaugcguaugucccaccuaaaggucggcucugcugccgacauccccaucaacaucucagagacggaucucagccugcugacggccacuguggucccgcccucgggccgggaggagcccuguuugcugaagcggcugcguaauggccacguggggauuucauucgugcccaaggagacgggggagcaccuggugcaugugaagaaaaauggccagcacguggccagcagccccaucccgguggugaucagccagucggaaauuggggaugccagucguguucgggucucuggucagggccuucacgaaggccacaccuuugagccugcagaguuuaucauugauacccgcgaugcaggcuauggugggcucagccuguccauugagggccccagcaagguggacaucaacacagaggaccuggaggacgggacgugcagggucaccuacugccccacagagccaggcaacuacaucaucaacaucaaguuugccgaccagcacgugccuggcagccccuucucugugaaggugacaggcgagggccgggugaaagagagcaucacccgcaggcgucgggcuccuucaguggccaacguugguagucauugugaccucagccugaaaaucccugaaauuagcauccaggauaugacagcccaggugaccagcccaucgggcaagacccaugaggccgagaucguggaaggggagaaccacaccuacugcauccgcuuuguucccgcugagaugggcacacacacagucagcgugaaguacaagggccagcacgugccugggagccccuuccaguucaccguggggccccuaggggaagggggagcccacaagguccgagcugggggcccuggccuggagagagcugaagcuggagugccagccgaauucaguaucuggacccgggaagcuggugcuggaggccuggccauugcugucgagggccccagcaaggcugagaucucuuuugaggaccgcaaggacggcuccugugguguggcuuaugugguccaggagccaggugacuacgaagucucagucaaguucaacgaggaacacauucccgacagccccuucguggugccuguggcuucuccgucuggcgacgcccgccgccucacuguuucuagccuucaggagucagggcuaaaggucaaccagccagccucuuuugcagucagccugaacggggccaagggggcgaucgaugccaaggugcacagccccucaggagcccuggaggagugcuaugucacagaaauugaccaagauaaguaugcugugcgcuucaucccucgggagaauggcguuuaccugauugacgucaaguucaacggcacccacaucccuggaagccccuucaagauccgaguuggggagccugggcauggaggggacccaggcuuggugucugcuuacggagcaggucuggaaggcggugucacagggaacccagcugaguucgucgugaacacgagcaaugcgggagcuggugcccugucggugaccauugacggccccuccaaggugaagauggauugccaggagugcccugagggcuaccgcgucaccuauacccccauggcaccuggcagcuaccucaucuccaucaaguacggcggccccuaccacauugggggcagccccuucaaggccaaagucacaggcccccgucucgucagcaaccacagccuccacgagacaucaucaguguuuguagacucucugaccaaggccaccugugccccccagcauggggccccggguccugggccugcugacgccagcaaggugguggccaagggccuggggcugagcaaggccuacguaggccagaagagcagcuucacaguagacugcagcaaagcaggcaacaacaugcugcugguggggguucauggcccaaggacccccugcgaggagauccuggugaagcacgugggcagccggcucuacagcguguccuaccugcucaaggacaagggggaguacacacugguggucaaauggggggacgagcacaucccaggcagccccuaccgcguuguggugcccugagucuggggcccgugccagccggcagcccccaagccugccccgcuacccaagcagccccgcccucuuccccucaaccccggcccaggccgcccuggccgcccgccugucacugcagccgccccugcccugugccgugcugcgcucaccugccuccccagccagccgcugaccucucggcuuucacuugggcagagggagccauuugguggcgcugcuugucuucuuugguucugggaggggugagggauggggguccuguacacaaccacccacuaguucucuucuccagccaagaggaauaaaguuuugcuuccauuaaaaaaaaaaaaaaaaaaaaaaaaaaaaaaaaaaaaaaaaaaaaaaa

SEQ ID NO: 8 - RNA, 8557 nt, Homo sapiens
  filamin A isoform 2 mRNA
auucgcguggaggcgcgucgcgcgcagcggacgccgacagaauccccgaggcgccuggcgcgggcgcgggcgcgaaggcgauccgggcgccaccccgcggucaucggucaccggucgcucucaggaacagcagcgcaaccucugcucccugccucgccucccgcgcgccuaggugccugcgacuuuaauuaaagggccguccccucgccgaggcugcagcaccgcccccccggcuucucgcgccucaaaaugaguagcucccacucucgggcgggccagagcgcagcaggcgcggcuccgggcggcggcgucgacacgcgggacgccgagaugccggccaccgagaaggaccuggcggaggacgcgccguggaagaagauccagcagaacacuuucacgcgcuggugcaacgagcaccugaagugcgugagcaagcgcaucgccaaccugcagacggaccugagcgacgggcugcggcuuaucgcgcuguuggaggugcucagccagaagaagaugcaccgcaagcacaaccagcggcccacuuuccgccaaaugcagcuugagaacgugucgguggcgcucgaguuccuggaccgcgagagcaucaaacugguguccaucgacagcaaggccaucguggacgggaaccugaagcugauccugggccucaucuggacccugauccugcacuacuccaucuccaugcccaugugggacgaggaggaggaugaggaggccaagaagcagacccccaagcagaggcuccugggcuggauccagaacaagcugccgcagcugcccaucaccaacuucagccgggacuggcagagcggccgggcccugggcgcccugguggacagcugugccccgggccuguguccugacugggacucuugggacgccagcaagcccguuaccaaugcgcgagaggccaugcagcaggcggaugacuggcugggcaucccccaggugaucacccccgaggagauuguggaccccaacguggacgagcacucugucaugaccuaccugucccaguuccccaaggccaagcugaagccaggggcucccuugcggcccaaacugaacccgaagaaagcccgugccuacgggccaggcaucgagcccacaggcaacauggugaagaagcgggcagaguucacuguggagaccagaagugcuggccagggagaggugcugguguacguggaggacccggccggacaccaggaggaggcaaaagugaccgccaauaacgacaagaaccgcaccuucuccgucugguacguccccgaggugacggggacucauaagguuacugugcucuuugcuggccagcacaucgccaagagccccuucgagguguacguggauaagucacagggugacgccagcaaagugacagcccaaggucccggccuggagcccaguggcaacaucgccaacaagaccaccuacuuugagaucuuuacggcaggagcuggcacgggcgaggucgagguugugauccaggaccccaugggacagaagggcacgguagagccucagcuggaggcccggggcgacagcacauaccgcugcagcuaccagcccaccauggagggcguccacaccgugcacgucacguuugccggcgugcccaucccucgcagccccuacacugucacuguuggccaagccuguaacccgagugccugccgggcgguuggccggggccuccagcccaagggugugcgggugaaggagacagcugacuucaagguguacacaaagggcgcuggcaguggggagcugaaggucaccgugaagggccccaagggagaggagcgcgugaagcagaaggaccugggggauggcguguauggcuucgaguauuaccccauggucccuggaaccuauaucgucaccaucacgugggguggucagaacaucgggcgcagucccuucgaagugaaggugggcaccgaguguggcaaucagaagguacgggccuggggcccugggcuggagggcggcgucguuggcaagucagcagacuuugugguggaggcuaucggggacgacgugggcacgcugggcuucucgguggaagggccaucgcaggcuaagaucgaaugugacgacaagggcgacggcuccugugaugugcgcuacuggccgcaggaggcuggcgaguaugccguucacgugcugugcaacagcgaagacauccgccucagccccuucauggcugacauccgugacgcgccccaggacuuccacccagacagggugaaggcacgugggccuggauuggagaagacagguguggccgucaacaagccagcagaguucacaguggaugccaagcacgguggcaaggccccacuucggguccaaguccaggacaaugaaggcugcccuguggaggcguuggucaaggacaacggcaauggcacuuacagcugcuccuacgugcccaggaagccggugaagcacacagccaugguguccuggggaggcgucagcauccccaacagccccuucagggugaaugugggagcuggcagccaccccaacaaggucaaaguauacggccccggaguagccaagacagggcucaaggcccacgagcccaccuacuucacuguggacugcgccgaggcuggccagggggacgucagcaucggcaucaagugugccccuggagugguaggccccgccgaagcugacaucgacuucgacaucauccgcaaugacaaugacaccuucacggucaaguacacgccccggggggcuggcagcuacaccauuaugguccucuuugcugaccaggccacgcccaccagccccauccgagucaagguggagcccucucaugacgccaguaaggugaaggccgagggcccuggccucagucgcacuggugucgagcuuggcaagcccacccacuucacaguaaaugccaaagcugcuggcaaaggcaagcuggacguccaguucucaggacucaccaagggggaugcagugcgagauguggacaucaucgaccaccaugacaacaccuacacagucaaguacacgccuguccagcaggguccaguaggcgucaaugucacuuauggaggggaucccaucccuaagagcccuuucucaguggcaguaucuccaagccuggaccucagcaagaucaaggugucuggccugggagagaagguggacguuggcaaagaccaggaguucacagucaaaucaaagggugcugguggucaaggcaaaguggcauccaagauugugggccccucgggugcagcggugcccugcaagguggagccaggccugggggcugacaacaguguggugcgcuuccugccccgugaggaagggcccuaugagguggaggugaccuaugacggcgugcccgugccuggcagccccuuuccucuggaagcuguggcccccaccaagccuagcaaggugaaggcguuugggccggggcugcagggaggcagugcgggcucccccgcccgcuucaccaucgacaccaagggcgccggcacagguggccugggccugacgguggagggccccugugaggcgcagcucgagugcuuggacaauggggauggcacauguuccguguccuacgugcccaccgagcccggggacuacaacaucaacauccucuucgcugacacccacaucccuggcuccccauucaaggcccacgugguucccugcuuugacgcauccaaagucaagugcucaggccccgggcuggagcgggccaccgcuggggaggugggccaauuccaaguggacugcucgagcgcgggcagcgcggagcugaccauugagaucugcucggaggcggggcuuccggccgagguguacauccaggaccacggugauggcacgcacaccauuaccuacauuccccucugccccggggccuacaccgucaccaucaaguacggcggccagcccgugcccaacuuccccagcaagcugcagguggaaccugcgguggacacuuccgguguccagugcuaugggccugguauugagggccagggugucuuccgugaggccaccacugaguucaguguggacgcccgggcucugacacagaccggagggccgcacgucaaggcccguguggccaaccccucaggcaaccugacggagaccuacguucaggaccguggcgauggcauguacaaaguggaguacacgccuuacgaggagggacugcacuccguggacgugaccuaugacggcagucccgugcccagcagccccuuccaggugcccgugaccgagggcugcgaccccucccgggugcguguccacgggccaggcauccaaaguggcaccaccaacaagcccaacaaguucacuguggagaccaggggagcuggcacgggcggccugggccuggcuguagagggccccuccgaggccaagauguccugcauggauaacaaggacggcagcugcucggucgaguacaucccuuaugaggcuggcaccuacagccucaacgucaccuaugguggccaucaagugccaggcaguccuuucaaggucccugugcaugaugugacagaugcguccaaggucaagugcucugggcccggccugagcccaggcaugguucgugccaaccucccucaguccuuccagguggacacaagcaaggcugguguggccccauugcaggucaaagugcaagggcccaaaggccugguggagccaguggacgugguagacaacgcugauggcacccagaccgucaauuaugugcccagccgagaagggcccuacagcaucucaguacuguauggagaugaagagguaccccggagccccuucaaggucaaggugcugccuacucaugaugccagcaaggugaaggccaguggccccgggcucaacaccacuggcgugccugccagccugcccguggaguucaccaucgaugcaaaggacgccggggagggccugcuggcuguccagaucacggaucccgaaggcaagccgaagaagacacacauccaagacaaccaugacggcacguauacaguggccuacgugccagacgugacaggucgcuacaccauccucaucaaguacgguggugacgagauccccuucuccccguaccgcgugcgugccgugcccaccggggacgccagcaagugcacugucacagugucaaucggaggucacgggcuaggugcuggcaucggccccaccauucagauuggggaggagacggugaucacuguggacacuaaggcggcaggcaaaggcaaagugacgugcaccgugugcacgccugauggcucagagguggauguggacgugguggagaaugaggacggcacuuucgacaucuucuacacggccccccagccgggcaaauacgucaucugugugcgcuuugguggcgagcacgugcccaacagccccuuccaagugacggcucuggcuggggaccagcccucggugcagcccccucuacggucucagcagcuggccccacaguacaccuacgcccagggcggccagcagacuugggccccggagaggccccuggugggugucaaugggcuggaugugaccagccugaggcccuuugaccuugucauccccuucaccaucaagaagggcgagaucacaggggagguucggaugcccucaggcaagguggcgcagcccaccaucacugacaacaaagacggcaccgugaccgugcgguaugcacccagcgaggcuggccugcacgagauggacauccgcuaugacaacaugcacaucccaggaagccccuugcaguucuauguggauuacgucaacuguggccaugucacugccuaugggccuggccucacccauggaguagugaacaagccugccaccuucaccgucaacaccaaggaugcaggagaggggggccugucucuggccauugagggcccguccaaagcagaaaucagcugcacugacaaccaggaugggacaugcagcguguccuaccugccugugcugccgggggacuacagcauucuagucaaguacaaugaacagcacgucccaggcagccccuucacugcucgggucacaggugacgacuccaugcguaugucccaccuaaaggucggcucugcugccgacauccccaucaacaucucagagacggaucucagccugcugacggccacuguggucccgcccucgggccgggaggagcccuguuugcugaagcggcugcguaauggccacguggggauuucauucgugcccaaggagacgggggagcaccuggugcaugugaagaaaaauggccagcacguggccagcagccccaucccgguggugaucagccagucggaaauuggggaugccagucguguucgggucucuggucagggccuucacgaaggccacaccuuugagccugcagaguuuaucauugauacccgcgaugcaggcuauggugggcucagccuguccauugagggccccagcaagguggacaucaacacagaggaccuggaggacgggacgugcagggucaccuacugccccacagagccaggcaacuacaucaucaacaucaaguuugccgaccagcacgugccuggcagccccuucucugugaaggugacaggcgagggccgggugaaagagagcaucacccgcaggcgucgggcuccuucaguggccaacguugguagucauugugaccucagccugaaaaucccugaaauuagcauccaggauaugacagcccaggugaccagcccaucgggcaagacccaugaggccgagaucguggaaggggagaaccacaccuacugcauccgcuuuguucccgcugagaugggcacacacacagucagcgugaaguacaagggccagcacgugccugggagccccuuccaguucaccguggggccccuaggggaagggggagcccacaagguccgagcugggggcccuggccuggagagagcugaagcuggagugccagccgaauucaguaucuggacccgggaagcuggugcuggaggccuggccauugcugucgagggccccagcaaggcugagaucucuuuugaggaccgcaaggacggcuccugugguguggcuuaugugguccaggagccaggugacuacgaagucucagucaaguucaacgaggaacacauucccgacagccccuucguggugccuguggcuucuccgucuggcgacgcccgccgccucacuguuucuagccuucaggagucagggcuaaaggucaaccagccagccucuuuugcagucagccugaacggggccaagggggcgaucgaugccaaggugcacagccccucaggagcccuggaggagugcuaugucacagaaauugaccaagauaaguaugcugugcgcuucaucccucgggagaauggcguuuaccugauugacgucaaguucaacggcacccacaucccuggaagccccuucaagauccgaguuggggagccugggcauggaggggacccaggcuuggugucugcuuacggagcaggucuggaaggcggugucacagggaacccagcugaguucgucgugaacacgagcaaugcgggagcuggugcccugucggugaccauugacggccccuccaaggugaagauggauugccaggagugcccugagggcuaccgcgucaccuauacccccauggcaccuggcagcuaccucaucuccaucaaguacggcggccccuaccacauugggggcagccccuucaaggccaaagucacaggcccccgucucgucagcaaccacagccuccacgagacaucaucaguguuuguagacucucugaccaaggccaccugugccccccagcauggggccccggguccugggccugcugacgccagcaaggugguggccaagggccuggggcugagcaaggccuacguaggccagaagagcagcuucacaguagacugcagcaaagcaggcaacaacaugcugcugguggggguucauggcccaaggacccccugcgaggagauccuggugaagcacgugggcagccggcucuacagcguguccuaccugcucaaggacaagggggaguacacacugguggucaaauggggggacgagcacaucccaggcagccccuaccgcguuguggugcccugagucuggggcccgugccagccggcagcccccaagccugccccgcuacccaagcagccccgcccucuuccccucaaccccggcccaggccgcccuggccgcccgccugucacugcagccgccccugcccugugccgugcugcgcucaccugccuccccagccagccgcugaccucucggcuuucacuugggcagagggagccauuugguggcgcugcuugucuucuuugguucugggaggggugagggauggggguccuguacacaaccacccacuaguucucuucuccagccaagaggaauaaaguuuugcuuccauuaaaaaaaaaaaaaaaaaaaaaaaaaaaaaaaaaaaaaaaaaaaaaaa

SEQ ID NO: 9 - RNA, 9534 nt, Homo sapiens
  filamin B mRNA
agagcagcaccggccguggcuccgguagcagcaaguucgaaccccgcucccgcuccgcuucgguucucgcuccuucggcccuugggccuccaaacaccaguccccggcagcucguugcgcauugcgcucuccccgccaccaggaugccgguaaccgagaaggaucuagcugaggacgcgccuuggaagaagauccagcagaacacguucacacgcuggugcaacgagcaccucaagugcgugaacaaacgcaucggcaaccugcagaccgaccugagcgacgggcugcggcucaucgcgcugcucgaggugcucagccagaagcgcauguaccgcaaguaccaucagcggcccaccuuucgccagaugcagcucgagaauguguccguggcgcucgaguuccuggaccgugagagcaucaagcucguguccaucgauagcaaagccauuguggaugggaaccugaagcucaucuugggucugguguggacgcugauccuccacuacuccaucuccaugcccgugugggaggaugaaggggaugaugaugccaagaagcagacgccaaagcagaggcugcugggguggauucagaacaagauccccuacuugcccaucaccaacuuuaaccagaacuggcaagacggcaaagcccugggagcccugguagacagcugugcuccaggucugugcccagacugggaauccugggacccgcagaagccuguggauaaugcacgagaagccaugcagcaggcagaugacuggcugggugucccacaggucaucacuccugaagaaaucauucacccggauguggacgagcacucaguuaugacuuaccugucccaguuccccaaagccaagcucaagccgggggcuccucucaaacccaaacucaacccgaagaaagccagggccuauggcagaggaaucgagcccacuggaaacauggugaagcagccagccaaguucacuguggacaccaucagcgccgggcaaggagacgugaugguguuuguugaggacccagaagggaacaaagaggaggcacaagugaccccugacagugacaagaacaagacauacucuguggaguaucugcccaaggucaccgggcuacacaaagucacaguccucuuugcaggacagcacaucuccaagagcccauuugaagugaguguugacaaggcccagggagaugccaguaaagucacugcaaaagguccaggguuggaagcuguagggaacaucgccaauaagcccaccuacuuugacaucuauacggcaggagcuggugugggugacauugguguggagguggaagauccccaggggaagaacaccguggaguugcucguggaagacaaaggaaaccagguguaucgauguguguacaaacccaugcagccuggcccucacguggucaagaucuucuuugcuggggacacuauuccuaagagucccuucguugugcagguuggggaagccugcaauccaaaugccugccgggccaguggccgaggccuacaacccaaaggcguccguauccgggagaccacagauuucaagguugacaccaaagcugcaggaaguggggagcucgguguaaccaugaaggguccuaagggucuggaggagcuggugaagcagaaagacuuucuggauggggucuacgcauucgaguauuaccccagcaccccggggagauacagcauugccaucacaugggggggacaccacauuccaaagagccccuuugaaguucaaguuggcccugaagcggguaugcagaaaguccgugcuuggggcccugggcuccauggugggauugucgggcggucagcggacuucgugguagaauccauuggcucugaaguggggucucugggguuugccauugaaggccccucucaggcaaagauugaguacaacgaccagaaugauggaucgugugaugucaaauacuggcccaaggagccuggcgaauaugcuguucacaucaugugugacgacgaagacaucaaggacagcccguacauggccuucauccacccagccacgggaggcuacaacccugaucugguucgagcauacgggccagguuuggagaaaucuggaugcauugucaacaaccuggccgaguucacuguggauccuaaggaugcuggaaaagcucccuuaaagauauuugcucaggauggggaaggccaacgcauugacauccagaugaagaaccggauggacggcacauaugcaugcucauacaccccggugaaggccaucaagcacaccauugcuguggucuggggaggcgugaacaucccgcacagccccuacagggucaacaucgggcaagguagccauccucagaaggucaaaguguuugggccagguguggagagaaguggucugaaggcaaaugaaccuacacacuucacgguggacuguacugaggcuggggaaggugaugucaguguuggcauuaagugugaugcccggguguuaagugaagaugaggaagacguggauuuugacauuauucacaaugccaaugauacguucacagucaaauaugugccuccugcugcugggcgauacacuaucaaaguucucuuugcaucucaggaaauccccgccagcccuuucagagucaaaguugacccuucccacgaugccagcaaagugaaggcagaaggcccagggcucagcaaagcagguguggaaaaugggaaaccgacccacuucacugucuacaccaagggggcugggaaagccccgcucaacgugcaguucaacagcccucuuccuggcgaugcagugaaggauuuggauaucaucgauaauuaugacuacucucacacgguuaaauauacacccacccaacagggcaacaugcagguucuggugacuuacgguggcgaucccaucccuaaaagcccuuucacuguggguguugcugcaccgcuggaucugagcaagauaaaacucaaugggcuggaaaacaggguggaaguugggaaggaucaggaguucaccguugauaccaggggggcaggaggccaggggaagcuggacgugacaauccucagccccucucggaaggucgugccaugccuagugacaccugugacaggccgggagaacagcacggccaaguucaucccucgggaggaggggcuguaugcuguagacgugaccuacgauggacacccugugcccgggagccccuacacaguggaggccucgcugccaccagaucccagcaaggugaaggcccacggucccggccucgaagguggucucgugggcaagccugccgaguucaccaucgauaccaaaggagcugguacuggaggucugggcuuaacgguggaagguccgugcgaggccaaaaucgagugcuccgacaauggugaugggaccugcuccgucucuuaccuucccacaaaacccggggaguacuucgucaacauccucuuugaagaaguccacauaccugggucucccuucaaagcugacauugaaaugcccuuugaccccucuaaagucguggcaucggggccaggucucgagcacgggaaggugggugaagcuggccuccuuagcgucgacugcucggaagcgggaccgggggcccugggccuggaagcugucucggacucgggaacaaaagccgaagucaguauucagaacaacaaagauggcaccuacgcggugaccuacgugccccugacggccggcauguacacguugaccaugaaguaugguggcgaacucgugccacacuuccccgcccgggucaagguggagcccgccguggacaccagcaggaucaaagucuuuggaccaggaauagaagggaaagauguguuccgggaagcuaccaccgacuuuacaguugacucucggccgcugacccagguugggggugaccacaucaaggcccacauugccaaccccucaggggccuccaccgagugcuuugucacagacaaugcggaugggaccuaccagguggaauacacacccuuugagaaaggucuccauguaguggaggugacauaugaugacgugccuaucccaaacagucccuucaagguggcugucacugaaggcugccagccaucuagggugcaagcccaaggaccuggauugaaagaggccuuuaccaacaagcccaaugucuucaccgugguuaccagaggcgcaggaauuggugggcuuggcauaacuguugagggaccaucagagucgaagauaaauugcagagacaacaaggauggcagcugcagugcugaguacauuccuuucgcaccgggggauuacgauguuaauaucacauauggaggagcccacauccccggcagccccuucaggguuccugugaaggauguuguggaccccagcaaggucaagauugccggccccgggcugggcucaggcguccgagcccguguccugcaguccuucacgguggacagcagcaaggcuggccuggcuccgcuggaagugaggguucugggcccacgagcugacgacacggauucccagucauggcgcagccccuugaaagcccuuucagaguucuuuaaaggugacccgaagggugacuuuaauaagacaggcuugguggagccagugaacgugguggacaauggagauggcacacacacaguaaccuacaccccaucucaggagggaccuuacauggucucaguuaaauaugcugaugaagagauuccucgcagucccuucaaggucaagguccuucccacauaugaugccagcaaagugacugccaguggccccggccuuaguuccuauggugugccugccagucuaccuguggacuuugcaauugaugcccgagaugccggggaaggccugcuugcuguucaaauaacggaccaagaaggaaaacccaaaagagccauuguccaugacaauaaagauggcacguaugcugucaccuacauccccgacaagacugggcgcuauaugauuggagucaccuacgggggugacgacaucccacuuucuccuuaucgcauccgagccacacagacgggugaugccagcaagugccuggccacggguccuggaaucgccuccacugugaaaacuggcgaagaaguaggcuuugugguugaugccaagacugccgggaaggguaaagugaccugcacgguucugaccccagauggcacugaggccgaggccgaugucauugagaaugaagauggaaccuaugacaucuucuacacagcugccaagccgggcacauaugugaucuaugugcgcuucggugguguugauauuccuaacagccccuucacugucauggccacagauggggaagucacagccguggaggaggcaccgguaaaugcaugucccccuggauucaggcccugggugaccgaagaggccuaugucccagugagugacaugaacggccugggauuuaagccuuuugaccuggucauuccguuugcugucaggaaaggagaaaucacuggagagguccacaugccuucugggaagacagccacaccugagauuguggacaacaaggacggcacggucacuguuagauaugcccccacugaggucgggcuccaugagaugcacaucaaauacaugggcagccacaucccugagagcccacuccaguucuacgugaacuaccccaacaguggaaguguuucugcauacgguccaggccucguguauggaguggccaacaaaacugccaccuucaccaucgucacagaggaugcaggagaagguggucuggacuuggcuauugagggccccucaaaagcagaaaucagcugcauugacaauaaagaugggacaugcacagugaccuaccugccgacucugccaggcgacuacagcauucuggucaaguacaaugacaagcacaucccuggcagccccuucacagccaagaucacagaugacagcaggcggugcucccaggugaaguugggcucagccgcugacuuccugcucgacaucagugagacugaccucagcagccugacggccagcauuaaggccccaucuggccgagacgagcccugucuccugaagaggcugcccaacaaccacauuggcaucuccuucaucccccgggaagugggcgaacaucuggucagcaucaagaaaaauggcaaccauguggccaacagccccgugucuaucauggugguccagucggagauuggugacgcccgccgagccaaagucuauggccgcggccugucagaaggccggacuuucgagaugucugacuucaucguggacacaagggaugcagguuaugguggcauauccuuggcgguggaaggccccagcaaaguggacauccagacggaggaccuggaagauggcaccugcaaagucuccuacuucccuaccgugccugggguuuauaucgucuccaccaaauucgcugacgagcacgugccugggagcccauuuaccgugaagaucaguggggagggaagagucaaagagagcaucacccgcaccagucgggccccguccguggccacugucgggagcauuugugaccugaaccugaaaaucccagaaaucaacagcagugauaugucggcccacgucaccagccccucuggccgugugacugaggcagagauugugcccauggggaagaacucacacugcguccgguuugugccccaggagaugggcgugcacacggucagcgucaaguaccgugggcagcacgucaccggcagccccuuccaguucaccguggggccacuuggugaaggaggcgcccacaaggugcgggcaggaggcccuggccuggagagaggagaagcgggagucccagcugaguucagcauuuggacccgggaagcaggcgcuggaggccucuccaucgcuguugagggccccaguaaggccgagauuacauucgaugaccauaaaaaugggucgugcgguguaucuuauauugcccaagagccugguaacuacgagguguccaucaaguucaaugaugagcacaucccggaaagccccuaccuggugccggucaucgcacccuccgacgacgcccgccgccucacuguuaugagccuucaggaaucgggauuaaaaguuaaccagccagcauccuuugcuauaagguugaauggcgcaaaaggcaagauugaugcaaaggugcacagccccucuggagccguggaggagugccacgugucugagcuggagccagauaaguaugcuguucgcuucaucccucaugagaaugguguccacaccaucgaugucaaguucaaugggagccacgugguuggaagccccuucaaagugcgcguuggggagccuggacaagcggggaacccugcccugguguccgccuauggcacgggacucgaagggggcaccacagguauccagucggaauucuuuauuaacaccacccgagcagguccagggacauuauccgucaccaucgaaggcccauccaagguuaaaauggauugccaggaaacaccugaaggguacaaagucauguacacccccauggcuccugguaacuaccugaucagcgucaaauacggugggcccaaccacaucgugggcagucccuucaaggccaaggugacaggccagcgucuaguuagcccuggcucagccaacgagaccucauccauccugguggagucagugaccaggucgucuacagagaccugcuauagcgccauucccaaggcauccucggacgccagcaaggugaccucuaagggggcagggcucucaaaggccuuugugggccagaagaguuccuuccugguggacugcagcaaagcuggcuccaacaugcugcugaucgggguccaugggcccaccacccccugcgaggaggucuccaugaagcauguaggcaaccagcaauacaacgucacauacgucgucaaggagaggggcgauuaugugcuggcugugaaguggggggaggaacacaucccuggcagcccuuuucaugucacagugccuuaaaacaguuuucucaaauccuggagagaguucuugugguugcuuuuguugcuuguuuguaauucauuuuauacaaagcccuccagccuguuuguggggcugaaaccccaucccuaaaauauugcuguuguaaaaugccuucagaaauaaguccuagacuggacucuugagggacauauuggagaaucuuaagaaaugcaagcuuguucagggggcugagaagauccugaguacacuaggugcaaaccagaacucuugguggaacagaccagccacugcagcagacagaccaggaacacaaugagacugacauuucaaaaaaacaaaacuggcuagccugagcugcugguucacucuucagcauuuaugaaacaaggcuaggggaagaugggcagagaaaaaggggacaccuaguuugguugucauuuggcaaaggagaugacuuaaaauccgcuuaaucucuuccaguguccguguuaauguauuuggcuauuagaucacuagcacugcuuuaccgcuccucaucgccaacacccccaugcucuguggccuucuuacacuucucagagggcagaguggcagccgggcacccuacagaaacucagagggcagaguggcagccaggcccacaugucucucaaguaccuguccccucgcucuggugauuauuucuugcagaaucaccacacgagaccaucccggcagucaugguuuugcuuuaguuuuccaaguccguuucagucccuuccuuggucugaagaaauucugcaguggcgagcaguuucccacuugccaaagaucccuuuuaaccaacacuagcccuuguuuuuaacacacgcuccagcccuucaucagccugggcagucuuaccaaaauguuuaaagugaucucagaggggcccauggauuaacgcccucaucccaagguccgucccaugacauaacacuccacacccgccccagccaacuucaugggucacuuuuucuggaaaauaaugaucuguacagacaggacagaaugaaacuccugcgggucuuuggccugaaaguugggaaugguugggggagagaagggcagcagcuuauugguggucuuuucaccauuggcagaaacagugagagcuguguggugcagaaauccagaaaugagguguagggaauuuugccugccuuccugcagaccugagcuggcuuuggaaugagguuaaagugucagggacguugccugagcccaaauguguaguguggucugggcaggcagaccuuuagguuuugcugcuuaguccugaggaaguggccacucuuguggcagguguaguaucuggggcgaguguuggggguaaaagcccacccuacagaaaguggaacagcccggagccugaugugaaaggaccacggguguuguaagcugggacacggaagccaaacuggaaucaaacgccgacuguaaauuguaucuuauaacuuauuaaauaaaacauuugcuccguaaa

SEQ ID NO: 10 - RNA, 45 nt, Artificial Sequence
  Synthetic oligonucleotide
gggugguuuuucucggugucucauccgaaggcuagaaacaguggg

SEQ ID NO: 11 - RNA, 63 nt, Artificial Sequence
  Synthetic oligonucleotide
ggagccccaggugggugguuuuucucggugucucauccgaaggcuagaaacaguggggcggcg

SEQ ID NO: 12 - RNA, 43 nt, Artificial Sequence
  Synthetic oligonucleotide
gguugaccuuuagcccugaccuccgaaggcuagaaacaguggg

SEQ ID NO: 13 - RNA, 65 nt, Artificial Sequence
  Synthetic oligonucleotide
gcaaaagaggcuggcugguugaccuuuagcccugaccuccgaaggcuagaaacaguggggcggcg

SEQ ID NO: 14 - RNA, 46 nt, Artificial Sequence
  Synthetic oligonucleotide
agauggaugcuuccuugcaucucacccgaaggcucauaacagugag

SEQ ID NO: 15 - RNA, 64 nt, Artificial Sequence
  Synthetic oligonucleotide
cggccaaggagauggaugcuuccuugcaucucacccgaaggcucauaacagugaggcggcgggc

SEQ ID NO: 16 - RNA, 43 nt, Artificial Sequence
  Synthetic oligonucleotide
gguuaacuuuuaaucccgauucccgaaggcucauaacagugag

SEQ ID NO: 17 - RNA, 63 nt, Artificial Sequence
  Synthetic oligonucleotide
ggaugcuggcugguuaacuuuuaaucccgauucccgaaggcucauaacagugaggcggcgggc

SEQ ID NO: 18 - RNA, 100 nt, Artificial Sequence
  Synthetic oligonucleotide
ggugucgagaagaggagaacaauaugcuaaauguuguucucgucuccucgacaccgggugguuuuucucggugucucauccgaaggcuagaaacaguggg

SEQ ID NO: 19 - RNA, 118 nt, Artificial Sequence
  Synthetic oligonucleotide
ggugucgagaagaggagaacaauaugcuaaauguuguucucgucuccucgacaccggagccccaggugggugguuuuucucggugucucauccgaaggcuagaaacaguggggcggcg

SEQ ID NO: 20 - RNA, 98 nt, Artificial Sequence
  Synthetic oligonucleotide
ggugucgagaagaggagaacaauaugcuaaauguuguucucgucuccucgacaccgguugaccuuuagcccugaccuccgaaggcuagaaacaguggg

SEQ ID NO: 21 - RNA, 120 nt, Artificial Sequence
  Synthetic oligonucleotide
ggugucgagaagaggagaacaauaugcuaaauguuguucucgucuccucgacaccgcaaaagaggcuggcugguugaccuuuagcccugaccuccgaaggcuagaaacaguggggcggcg

SEQ ID NO: 22 - RNA, 101 nt, Artificial Sequence
  Synthetic oligonucleotide
ggugucgagaagaggagaacaauaugcuaaauguuguucucgucuccucgacaccagauggaugcuuccuugcaucucacccgaaggcucauaacagugag

SEQ ID NO: 23 - RNA, 119 nt, Artificial Sequence
  Synthetic oligonucleotide
ggugucgagaagaggagaacaauaugcuaaauguuguucucgucuccucgacacccggccaaggagauggaugcuuccuugcaucucacccgaaggcucauaacagugaggcggcgggc

SEQ ID NO: 24 - RNA, 98 nt, Artificial Sequence
  Synthetic oligonucleotide
ggugucgagaagaggagaacaauaugcuaaauguuguucucgucuccucgacaccgguuaacuuuuaaucccgauucccgaaggcucauaacagugag

SEQ ID NO: 25 - RNA, 118 nt, Artificial Sequence
  Synthetic oligonucleotide
ggugucgagaagaggagaacaauaugcuaaauguuguucucgucuccucgacaccggaugcuggcugguuaacuuuuaaucccgauucccgaaggcucauaacagugaggcggcgggc

SEQ ID NO: 26 - RNA, 55 nt, Artificial Sequence
  Synthetic oligonucleotide
ggugucgagaagaggagaacaauaugcuaaauguuguucucgucuccucgacacc

SEQ ID NO: 27 - protein, 2639 aa, Homo sapiens
  filamin-A isoform 1 unmodified
MSSSHSRAGQSAAGAAPGGGVDTRDAEMPATEKDLAEDAPWKKIQQNTFTRWCNEHLKCVSKRIANLQTDLSDGLRLIALLEVLSQKKMHRKHNQRPTFRQMQLENVSVALEFLDRESIKLVSIDSKAIVDGNLKLILGLIWTLILHYSISMPMWDEEEDEEAKKQTPKQRLLGWIQNKLPQLPITNFSRDWQSGRALGALVDSCAPGLCPDWDSWDASKPVTNAREAMQQADDWLGIPQVITPEEIVDPNVDEHSVMTYLSQFPKAKLKPGAPLRPKLNPKKARAYGPGIEPTGNMVKKRAEFTVETRSAGQGEVLVYVEDPAGHQEEAKVTANNDKNRTFSVWYVPEVTGTHKVTVLFAGQHIAKSPFEVYVDKSQGDASKVTAQGPGLEPSGNIANKTTYFEIFTAGAGTGEVEVVIQDPMGQKGTVEPQLEARGDSTYRCSYQPTMEGVHTVHVTFAGVPIPRSPYTVTVGQACNPSACRAVGRGLQPKGVRVKETADFKVYTKGAGSGELKVTVKGPKGEERVKQKDLGDGVYGFEYYPMVPGTYIVTITWGGQNIGRSPFEVKVGTECGNQKVRAWGPGLEGGVVGKSADFVVEAIGDDVGTLGFSVEGPSQAKIECDDKGDGSCDVRYWPQEAGEYAVHVLCNSEDIRLSPFMADIRDAPQDFHPDRVKARGPGLEKTGVAVNKPAEFTVDAKHGGKAPLRVQVQDNEGCPVEALVKDNGNGTYSCSYVPRKPVKHTAMVSWGGVSIPNSPFRVNVGAGSHPNKVKVYGPGVAKTGLKAHEPTYFTVDCAEAGQGDVSIGIKCAPGVVGPAEADIDFDIIRNDNDTFTVKYTPRGAGSYTIMVLFADQATPTSPIRVKVEPSHDASKVKAEGPGLSRTGVELGKPTHFTVNAKAAGKGKLDVQFSGLTKGDAVRDVDIIDHHDNTYTVKYTPVQQGPVGVNVTYGGDPIPKSPFSVAVSPSLDLSKIKVSGLGEKVDVGKDQEFTVKSKGAGGQGKVASKIVGPSGAAVPCKVEPGLGADNSVVRFLPREEGPYEVEVTYDGVPVPGSPFPLEAVAPTKPSKVKAFGPGLQGGSAGSPARFTIDTKGAGTGGLGLTVEGPCEAQLECLDNGDGTCSVSYVPTEPGDYNINILFADTHIPGSPFKAHVVPCFDASKVKCSGPGLERATAGEVGQFQVDCSSAGSAELTIEICSEAGLPAEVYIQDHGDGTHTITYIPLCPGAYTVTIKYGGQPVPNFPSKLQVEPAVDTSGVQCYGPGIEGQGVFREATTEFSVDARALTQTGGPHVKARVANPSGNLTETYVQDRGDGMYKVEYTPYEEGLHSVDVTYDGSPVPSSPFQVPVTEGCDPSRVRVHGPGIQSGTTNKPNKFTVETRGAGTGGLGLAVEGPSEAKMSCMDNKDGSCSVEYIPYEAGTYSLNVTYGGHQVPGSPFKVPVHDVTDASKVKCSGPGLSPGMVRANLPQSFQVDTSKAGVAPLQVKVQGPKGLVEPVDVVDNADGTQTVNYVPSREGPYSISVLYGDEEVPRSPFKVKVLPTHDASKVKASGPGLNTTGVPASLPVEFTIDAKDAGEGLLAVQITDPEGKPKKTHIQDNHDGTYTVAYVPDVTGRYTILIKYGGDEIPFSPYRVRAVPTGDASKCTVTGAGIGPTIQIGEETVITVDTKAAGKGKVTCTVCTPDGSEVDVDVVENEDGTFDIFYTAPQPGKYVICVRFGGEHVPNSPFQVTALAGDQPSVQPPLRSQQLAPQYTYAQGGQQTWAPERPLVGVNGLDVTSLRPFDLVIPFTIKKGEITGEVRMPSGKVAQPTITDNKDGTVTVRYAPSEAGLHEMDIRYDNMHIPGSPLQFYVDYVNCGHVTAYGPGLTHGVVNKPATFTVNTKDAGEGGLSLAIEGPSKAEISCTDNQDGTCSVSYLPVLPGDYSILVKYNEQHVPGSPFTARVTGDDSMRMSHLKVGSAADIPINISETDLSLLTATVVPPSGREEPCLLKRLRNGHVGISFVPKETGEHLVHVKKNGQHVASSPIPVVISQSEIGDASRVRVSGQGLHEGHTFEPAEFIIDTRDAGYGGLSLSIEGPSKVDINTEDLEDGTCRVTYCPTEPGNYIINIKFADQHVPGSPFSVKVTGEGRVKESITRRRRAPSVANVGSHCDLSLKIPEISIQDMTAQVTSPSGKTHEAEIVEGENHTYCIRFVPAEMGTHTVSVKYKGQHVPGSPFQFTVGPLGEGGAHKVRAGGPGLERAEAGVPAEFSIWTREAGAGGLAIAVEGPSKAEISFEDRKDGSCGVAYVVQEPGDYEVSVKFNEEHIPDSPFVVPVASPSGDARRLTVSSLQESGLKVNQPASFAVSLNGAKGAIDAKVHSPSGALEECYVTEIDQDKYAVRFIPRENGVYLIDVKFNGTHIPGSPFKIRVGEPGHGGDPGLVSAYGAGLEGGVTGNPAEFVVNTSNAGAGALSVTIDGPSKVKMDCQECPEGYRVTYTPMAPGSYLISIKYGGPYHIGGSPFKAKVTGPRLVSNHSLHETSSVFVDSLTKATCAPQHGAPGPGPADASKVVAKGLGLSKAYVGQKSSFTVDCSKAGNNMLLVGVHGPRTPCEEILVKHVGSRLYSVSYLLKDKGEYTLVVKWGDEHIPGSPYRVVVP

SEQ ID NO: 28 - protein, 2647 aa, Homo sapiens
  filamin-A isoform 2 unmodified
MSSSHSRAGQSAAGAAPGGGVDTRDAEMPATEKDLAEDAPWKKIQQNTFTRWCNEHLKCVSKRIANLQTDLSDGLRLIALLEVLSQKKMHRKHNQRPTFRQMQLENVSVALEFLDRESIKLVSIDSKAIVDGNLKLILGLIWTLILHYSISMPMWDEEEDEEAKKQTPKQRLLGWIQNKLPQLPITNFSRDWQSGRALGALVDSCAPGLCPDWDSWDASKPVTNAREAMQQADDWLGIPQVITPEEIVDPNVDEHSVMTYLSQFPKAKLKPGAPLRPKLNPKKARAYGPGIEPTGNMVKKRAEFTVETRSAGQGEVLVYVEDPAGHQEEAKVTANNDKNRTFSVWYVPEVTGTHKVTVLFAGQHIAKSPFEVYVDKSQGDASKVTAQGPGLEPSGNIANKTTYFEIFTAGAGTGEVEVVIQDPMGQKGTVEPQLEARGDSTYRCSYQPTMEGVHTVHVTFAGVPIPRSPYTVTVGQACNPSACRAVGRGLQPKGVRVKETADFKVYTKGAGSGELKVTVKGPKGEERVKQKDLGDGVYGFEYYPMVPGTYIVTITWGGQNIGRSPFEVKVGTECGNQKVRAWGPGLEGGVVGKSADFVVEAIGDDVGTLGFSVEGPSQAKIECDDKGDGSCDVRYWPQEAGEYAVHVLCNSEDIRLSPFMADIRDAPQDFHPDRVKARGPGLEKTGVAVNKPAEFTVDAKHGGKAPLRVQVQDNEGCPVEALVKDNGNGTYSCSYVPRKPVKHTAMVSWGGVSIPNSPFRVNVGAGSHPNKVKVYGPGVAKTGLKAHEPTYFTVDCAEAGQGDVSIGIKCAPGVVGPAEADIDFDIIRNDNDTFTVKYTPRGAGSYTIMVLFADQATPTSPIRVKVEPSHDASKVKAEGPGLSRTGVELGKPTHFTVNAKAAGKGKLDVQFSGLTKGDAVRDVDIIDHHDNTYTVKYTPVQQGPVGVNVTYGGDPIPKSPFSVAVSPSLDLSKIKVSGLGEKVDVGKDQEFTVKSKGAGGQGKVASKIVGPSGAAVPCKVEPGLGADNSVVRFLPREEGPYEVEVTYDGVPVPGSPFPLEAVAPTKPSKVKAFGPGLQGGSAGSPARFTIDTKGAGTGGLGLTVEGPCEAQLECLDNGDGTCSVSYVPTEPGDYNINILFADTHIPGSPFKAHVVPCFDASKVKCSGPGLERATAGEVGQFQVDCSSAGSAELTIEICSEAGLPAEVYIQDHGDGTHTITYIPLCPGAYTVTIKYGGQPVPNFPSKLQVEPAVDTSGVQCYGPGIEGQGVFREATTEFSVDARALTQTGGPHVKARVANPSGNLTETYVQDRGDGMYKVEYTPYEEGLHSVDVTYDGSPVPSSPFQVPVTEGCDPSRVRVHGPGIQSGTTNKPNKFTVETRGAGTGGLGLAVEGPSEAKMSCMDNKDGSCSVEYIPYEAGTYSLNVTYGGHQVPGSPFKVPVHDVTDASKVKCSGPGLSPGMVRANLPQSFQVDTSKAGVAPLQVKVQGPKGLVEPVDVVDNADGTQTVNYVPSREGPYSISVLYGDEEVPRSPFKVKVLPTHDASKVKASGPGLNTTGVPASLPVEFTIDAKDAGEGLLAVQITDPEGKPKKTHIQDNHDGTYTVAYVPDVTGRYTILIKYGGDEIPFSPYRVRAVPTGDASKCTVTVSIGGHGLGAGIGPTIQIGEETVITVDTKAAGKGKVTCTVCTPDGSEVDVDVVENEDGTFDIFYTAPQPGKYVICVRFGGEHVPNSPFQVTALAGDQPSVQPPLRSQQLAPQYTYAQGGQQTWAPERPLVGVNGLDVTSLRPFDLVIPFTIKKGEITGEVRMPSGKVAQPTITDNKDGTVTVRYAPSEAGLHEMDIRYDNMHIPGSPLQFYVDYVNCGHVTAYGPGLTHGVVNKPATFTVNTKDAGEGGLSLAIEGPSKAEISCTDNQDGTCSVSYLPVLPGDYSILVKYNEQHVPGSPFTARVTGDDSMRMSHLKVGSAADIPINISETDLSLLTATVVPPSGREEPCLLKRLRNGHVGISFVPKETGEHLVHVKKNGQHVASSPIPVVISQSEIGDASRVRVSGQGLHEGHTFEPAEFIIDTRDAGYGGLSLSIEGPSKVDINTEDLEDGTCRVTYCPTEPGNYIINIKFADQHVPGSPFSVKVTGEGRVKESITRRRRAPSVANVGSHCDLSLKIPEISIQDMTAQVTSPSGKTHEAEIVEGENHTYCIRFVPAEMGTHTVSVKYKGQHVPGSPFQFTVGPLGEGGAHKVRAGGPGLERAEAGVPAEFSIWTREAGAGGLAIAVEGPSKAEISFEDRKDGSCGVAYVVQEPGDYEVSVKFNEEHIPDSPFVVPVASPSGDARRLTVSSLQESGLKVNQPASFAVSLNGAKGAIDAKVHSPSGALEECYVTEIDQDKYAVRFIPRENGVYLIDVKFNGTHIPGSPFKIRVGEPGHGGDPGLVSAYGAGLEGGVTGNPAEFVVNTSNAGAGALSVTIDGPSKVKMDCQECPEGYRVTYTPMAPGSYLISIKYGGPYHIGGSPFKAKVTGPRLVSNHSLHETSSVFVDSLTKATCAPQHGAPGPGPADASKVVAKGLGLSKAYVGQKSSFTVDCSKAGNNMLLVGVHGPRTPCEEILVKHVGSRLYSVSYLLKDKGEYTLVVKWGDEHIPGSPYRVVVP

SEQ ID NO: 29 - protein, 2633 aa, Homo sapiens
  Filamin B isoform 1 unmodified
MPVTEKDLAEDAPWKKIQQNTFTRWCNEHLKCVNKRIGNLQTDLSDGLRLIALLEVLSQKRMYRKYHQRPTFRQMQLENVSVALEFLDRESIKLVSIDSKAIVDGNLKLILGLVWTLILHYSISMPVWEDEGDDDAKKQTPKQRLLGWIQNKIPYLPITNFNQNWQDGKALGALVDSCAPGLCPDWESWDPQKPVDNAREAMQQADDWLGVPQVITPEEIIHPDVDEHSVMTYLSQFPKAKLKPGAPLKPKLNPKKARAYGRGIEPTGNMVKQPAKFTVDTISAGQGDVMVFVEDPEGNKEEAQVTPDSDKNKTYSVEYLPKVTGLHKVTVLFAGQHISKSPFEVSVDKAQGDASKVTAKGPGLEAVGNIANKPTYFDIYTAGAGVGDIGVEVEDPQGKNTVELLVEDKGNQVYRCVYKPMQPGPHVVKIFFAGDTIPKSPFVVQVGEACNPNACRASGRGLQPKGVRIRETTDFKVDTKAAGSGELGVTMKGPKGLEELVKQKDFLDGVYAFEYYPSTPGRYSIAITWGGHHIPKSPFEVQVGPEAGMQKVRAWGPGLHGGIVGRSADFVVESIGSEVGSLGFAIEGPSQAKIEYNDQNDGSCDVKYWPKEPGEYAVHIMCDDEDIKDSPYMAFIHPATGGYNPDLVRAYGPGLEKSGCIVNNLAEFTVDPKDAGKAPLKIFAQDGEGQRIDIQMKNRMDGTYACSYTPVKAIKHTIAVVWGGVNIPHSPYRVNIGQGSHPQKVKVFGPGVERSGLKANEPTHFTVDCTEAGEGDVSVGIKCDARVLSEDEEDVDFDIIHNANDTFTVKYVPPAAGRYTIKVLFASQEIPASPFRVKVDPSHDASKVKAEGPGLSKAGVENGKPTHFTVYTKGAGKAPLNVQFNSPLPGDAVKDLDIIDNYDYSHTVKYTPTQQGNMQVLVTYGGDPIPKSPFTVGVAAPLDLSKIKLNGLENRVEVGKDQEFTVDTRGAGGQGKLDVTILSPSRKVVPCLVTPVTGRENSTAKFIPREEGLYAVDVTYDGHPVPGSPYTVEASLPPDPSKVKAHGPGLEGGLVGKPAEFTIDTKGAGTGGLGLTVEGPCEAKIECSDNGDGTCSVSYLPTKPGEYFVNILFEEVHIPGSPFKADIEMPFDPSKVVASGPGLEHGKVGEAGLLSVDCSEAGPGALGLEAVSDSGTKAEVSIQNNKDGTYAVTYVPLTAGMYTLTMKYGGELVPHFPARVKVEPAVDTSRIKVFGPGIEGKDVFREATTDFTVDSRPLTQVGGDHIKAHIANPSGASTECFVTDNADGTYQVEYTPFEKGLHVVEVTYDDVPIPNSPFKVAVTEGCQPSRVQAQGPGLKEAFTNKPNVFTVVTRGAGIGGLGITVEGPSESKINCRDNKDGSCSAEYIPFAPGDYDVNITYGGAHIPGSPFRVPVKDVVDPSKVKIAGPGLGSGVRARVLQSFTVDSSKAGLAPLEVRVLGPRADDTDSQSWRSPLKALSEFFKGDPKGDFNKTGLVEPVNVVDNGDGTHTVTYTPSQEGPYMVSVKYADEEIPRSPFKVKVLPTYDASKVTASGPGLSSYGVPASLPVDFAIDARDAGEGLLAVQITDQEGKPKRAIVHDNKDGTYAVTYIPDKTGRYMIGVTYGGDDIPLSPYRIRATQTGDASKCLATGPGIASTVKTGEEVGFVVDAKTAGKGKVTCTVLTPDGTEAEADVIENEDGTYDIFYTAAKPGTYVIYVRFGGVDIPNSPFTVMATDGEVTAVEEAPVNACPPGFRPWVTEEAYVPVSDMNGLGFKPFDLVIPFAVRKGEITGEVHMPSGKTATPEIVDNKDGTVTVRYAPTEVGLHEMHIKYMGSHIPESPLQFYVNYPNSGSVSAYGPGLVYGVANKTATFTIVTEDAGEGGLDLAIEGPSKAEISCIDNKDGTCTVTYLPTLPGDYSILVKYNDKHIPGSPFTAKITDDSRRCSQVKLGSAADFLLDISETDLSSLTASIKAPSGRDEPCLLKRLPNNHIGISFIPREVGEHLVSIKKNGNHVANSPVSIMVVQSEIGDARRAKVYGRGLSEGRTFEMSDFIVDTRDAGYGGISLAVEGPSKVDIQTEDLEDGTCKVSYFPTVPGVYIVSTKFADEHVPGSPFTVKISGEGRVKESITRTSRAPSVATVGSICDLNLKIPEINSSDMSAHVTSPSGRVTEAEIVPMGKNSHCVRFVPQEMGVHTVSVKYRGQHVTGSPFQFTVGPLGEGGAHKVRAGGPGLERGEAGVPAEFSIWTREAGAGGLSIAVEGPSKAEITFDDHKNGSCGVSYIAQEPGNYEVSIKFNDEHIPESPYLVPVIAPSDDARRLTVMSLQESGLKVNQPASFAIRLNGAKGKIDAKVHSPSGAVEECHVSELEPDKYAVRFIPHENGVHTIDVKFNGSHVVGSPFKVRVGEPGQAGNPALVSAYGTGLEGGTTGIQSEFFINTTRAGPGTLSVTIEGPSKVKMDCQETPEGYKVMYTPMAPGNYLISVKYGGPNHIVGSPFKAKVTGQRLVSPGSANETSSILVESVTRSSTETCYSAIPKASSDASKVTSKGAGLSKAFVGQKSSFLVDCSKAGSNMLLIGVHGPTTPCEEVSMKHVGNQQYNVTYVVKERGDYVLAVKWGEEHIPGSPFHVTVP

SEQ ID NO: 30 - protein, 421 aa, Artificial Sequence
  fusion protein
MDAQTRRRERRAEKQAQWKAANPLLVGVSAKPVNRPILSGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEGSRSYTQAGVQWCNHGSLQPRPPGLLSDPSTSTFQGAGTTEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 31 - protein, 401 aa, Artificial Sequence
  fusion protein
MDAQTRRRERRAEKQAQWKAANPLLVGVSAKPVNRPILSGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 32 - protein, 565 aa, Artificial Sequence
  fusion protein
MGPGSDKDCEMKRTTLDSPLGKLELSGCEQGLHEIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKRGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEGSRSYTQAGVQWCNHGSLQPRPPGLLSDPSTSTFQGAGTTEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 33 - protein, 545 aa, Artificial Sequence
  fusion protein
MGPGSDKDCEMKRTTLDSPLGKLELSGCEQGLHEIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKRGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 34 - protein, 362 aa, Artificial Sequence
  ADAR1 catalytic domain
GRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 35 - protein, 382 aa, Artificial Sequence
  ADAR2 catalytic domain
GTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEGSRSYTQAGVQWCNHGSLQPRPPGLLSDPSTSTFQGAGTTEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 36 - protein, 529 aa, Artificial Sequence
  fusion protein
MSPKKKRKVGSASNFTQFVLVDNGGTGDVTVAPSNFANGVAEWISSNSRSQAYKVTCSVRQSSAQNRKYTIKVEVPKVATQTVGGEELPVAGWRSYLNMELTIPIFATNSDCELIVKAMQGLLKDGNPIPSAIAANSGIYVIATAAAGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEGSRSYTQAGVQWCNHGSLQPRPPGLLSDPSTSTFQGAGTTEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 37 - protein, 521 aa, Artificial Sequence
  fusion protein
MGSASNFTQFVLVDNGGTGDVTVAPSNFANGVAEWISSNSRSQAYKVTCSVRQSSAQNRKYTIKVEVPKVATQTVGGEELPVAGWRSYLNMELTIPIFATNSDCELIVKAMQGLLKDGNPIPSAIAANSGIYVIATAAAGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEGSRSYTQAGVQWCNHGSLQPRPPGLLSDPSTSTFQGAGTTEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 38 - protein, 509 aa, Artificial Sequence
  fusion protein
MSPKKKRKVGSASNFTQFVLVDNGGTGDVTVAPSNFANGVAEWISSNSRSQAYKVTCSVRQSSAQNRKYTIKVEVPKVATQTVGGEELPVAGWRSYLNMELTIPIFATNSDCELIVKAMQGLLKDGNPIPSAIAANSGIYVIATAAAGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 39 - protein, 501 aa, Artificial Sequence
  fusion protein
MGSASNFTQFVLVDNGGTGDVTVAPSNFANGVAEWISSNSRSQAYKVTCSVRQSSAQNRKYTIKVEVPKVATQTVGGEELPVAGWRSYLNMELTIPIFATNSDCELIVKAMQGLLKDGNPIPSAIAANSGIYVIATAAAGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 40 - DNA, 23 bp, Artificial Sequence
  Complement of gRNA1
ctgtttctagtcttcaggtgagg

SEQ ID NO: 41 - DNA, 23 bp, Artificial Sequence
  Complement of gRNA2
gctacctgggtgtgtgattgtgg

SEQ ID NO: 42 - DNA, 20 bp, Artificial Sequence
  Genotyping primer forward
tctggatggtaggcttctgc

SEQ ID NO: 43 - DNA, 21 bp, Artificial Sequence
  Genotyping primer reverse
ctggaagcatagcagatgtgg

SEQ ID NO: 44 - DNA, 23 bp, Artificial Sequence
  F2
gtctgggtgttagtcagttagtc

SEQ ID NO: 45 - DNA, 23 bp, Artificial Sequence
  R2
cactctcccatgccactatctgc

SEQ ID NO: 46 - DNA, 23 bp, Artificial Sequence
  F3
gtctgggtgttagtcagttagtc

SEQ ID NO: 47 - DNA, 23 bp, Artificial Sequence
  R4
cgtctacccagtaccagatgttg

SEQ ID NO: 48 - DNA, 9705 bp, Artificial Sequence
  linearized homologous recombination construct
tagaggtaccggcggccgctctagaactagtggatccccgtaccgtttaaacgcctcgactctagtggatctgcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagagatcgcctgacacgatttcctgcacaggcttgagccatatactcatacatcgcatcttggccacgttttccacgggtttcaaaattaatctcaagttctacgcttaacgctttcgcctgttcccagttattaatatattcaacgctagaactcccctcagcgaagggaaggctgagcactacacgcgaagcaccatcaccgaaccttttgataaactcttccgttccgacttgctccatcaacggttcagtgagacttaaacctaactctttcttaatagtttcggcattatccacttttagtgcgagaaccttcgtcagtcctggatacgtcactttgaccacgcctccagcttttccagagagcgggttttcattatctacagagtatcccgcagcgtcgtatttattgtcggtactataaaaccctttccaatcatcgtcataatttccttgtgtaccagattttggcttttgtatacctttttgaatggaatctacataaccaggtttagtcccgtggtacgaagaaaagttttccatcacaaaagatttagaagaatcaacaacatcatcaggatccatggcgaggacctgcagggtcgctcggtgttcgaggccacacgcgtcaccttaatatgcgaagtggacctgggaccgcgccgccccgactgcatctgcgtgttcgaattcgccaatgacaagacgctgggcggggtttgctcgacattgggtggaaacattccaggcctgggtggagaggctttttgcttcctcttgcaaaaccacactgctcgacattgggtggaaacattccaggcctgggtggagaggctttttgcttcctcttgaaaaccacactgctcgaccgcgcttcggtacgggctgcaggaattcgatatcaagctttcagaaggcccaaatcattagctagatgatacagggcaggcataaaggaagttcatggcagcatgggcagcaggcaactagggtaatagctgaagttgctatctgagtctcatgtagccagtgtgagaccaaggtcgaaaagggggcttggtggcaggctggcaagggggccccttaaacttggtcctcatataagtttggtgttcctagctgctttccttgtgggaggctgggctgcaggggtgctctggctgccaggccttacaatccacagctcccaagcctccacagctgagccccagatcataacattactgtcctttctcttttcttgggcactatggggccctagaggctgctcagatgctctacccttgcaaccctgcagtggtggcttgggactcctgtctcctgttccttgggtgctgccactgctttgccaagcctgctgtgatgcactgctaacccttctacttccctgctgcttgctcttgcctgtgccaggctgtgctatgctgcctctcattgcaagcctctggttgagcaagcgttctcagttgtcaggagactccagtttcaccttcaggcctcggggttgggtagggaggaaagtttcacatcccccacactgcagcaggactctgcctcacttcatccacacatagcactggggcccagggccagaccacgggtgtaggtagccatgggcctggccagagtgccggtataagttagcataggccatgataccagctgaggctgagttggcagaggaggtagcagtaaatgcttgtgctgggattggatggtcccttacttgtcttcccaggtatctgggtaccacttgttggtcttctctcataaaggggcttgaggtggtcaggagaatccttcctgccttctgagaaaccactcaccatcgtagcttggggtatgggttgggctagagattggggctggctgtgcttggttgtagcagtaagaggcctggatgggtagcaagctgatgggggtagccaaaatgttcggctcacctgctggctcccctctggggattcactctgctttaccaggtgctggcattggccccaccatccagattggggaggagacggtgattactgtggacacaaaagcagcaggcaaaggcaaagtgacttgtactgtgtgcacacctgatggctcagaggtagacgtggacgtggtggagaatgaggatggcacctttgacatcttctacacagctccccaaccgggcaaatatgtcatctgtgtgcgcttcggtggcgagcatgtgcccaacagccccttccaagttacagtgagaagaggccaaggatgggtgggcagggagatctgggaaagtcctagcctgtccttccaggtccctttgtgacaacagtctccaaccctctcactttgccccacaggctttggctggggaccaaccaacagtgcagaccccattaaggtctcagcagctggctccacagtataactatcctcagggtagccagcaaacctgggtaaggcctgggcctggtctattattcaatttgtctaacccatagggacacttgaagctggccacctggatgtctcatgtggccctgagaaaccccatatcttctcatagcccctcacctgattcttctgtctctattgcagattccagagaggcccatggtgggcgttaatgggctggatgtgaccagcctgaggccctttgatcttgtcatccccttcactatcaagaagggggagatcactggtgagtgagggctaggaagaaacttggaaactagtgatggccagggtgttccaatctcctggcctgacattgaaaccactgctctgtaggggaagttcgaatgccctcaggcaaggtggcccagccttccattactgataacaaagatggcactgttactgtacgttactcacccagtgaagctggcctgcatgaaatggacattcgctatgacaatatgcatatcccaggtgggtgtattgcctcttgtccactccatcctaggcccgtgttgctatcatctcaggaagaacaggctagggctgggttataggtcagttagtagagcctagcatgcatcaggctctgaataaactaggtatcatggcacatgcctgtaatcatagccctcgaggcaggaggatcagtggttcaaggtcatccttagcttcatagtaagttcaaagccagccagagaaacataaagaccctgtttcaaaaacagaaccaaaatgactaacaaatgaatgagtctggtattccatgtggcttctattctggtctcaggaagccctctgcagttctatgttgattatgtcaactgtggccacatcactgcttatggtcctggccttacccatggagtggtcaacaaacctgccaccttcactgtcaacaccaaggatgcaggagagggtaaggagacgctctaggtttgacctgcccaagttgtcctccattggctgagaactgtatgtgggagatgatcctgaatggtgttttctaccttaccttcatgctgagccatcaagtcaactcacccacaaaggttcagcaatgtgtctctgggcacagttctcactcttatctgtgatatttttcagggggcttgtctctggccattgagggtccatctaaagcagaaatcagttgcactgacaaccaggatggaacatgcagtgtctcttacctgcctgtactgcctggtgactatagcatcctagttaagtacaatgatcaacacatcccaggcagtccctttactgccagagtaacaggtacggagcatcgggagtaatgacaggaacaggtggcatgaggtcacagtctctttgggctggatctatagtggctgatggcttgcacccttaccaggtgacgattccatgcgtatgtcccacctaaaggtgggttctgctgctgatatccccatcaatatctcagaaacagaccttagcctactcacagccactgtggtgccaccttcgggtcgagaggaaccctgtctgctgaaacgtttgcgaaatggccacgtgggtaagaggtcaagagggagcaaaagtttctgtgaaagagggaaagccaggtgtggtgactgcccatagtcctaatacttgggaggtgtaagtagaagaatcaggggaaccaatcattggctacataatgagttaaagtcattttagcctgtaataaataccctgtgttcagacaaagggagcaaggaagaagattgtgggcccacatcttgttgatagccctatcctgtgcctagggatttccttcgtgcccaaggagacaggggagcacctggtacatgtgaagaagaatggccagcatgtggcaagcagtcccatcccagtagtgatcagccagtcggagataggtgatgccagccgtgtgagggtctctggtcaaggtcttcatgaaggtcatacctttgagcctgcagagtttattattgacaccagagatgcaggtaggcagtgtggtctgctagttggggcaatacagctagctttccttggcattctggcttatagctgctatctatttcccaggctacggtgggcttagtctgtccattgagggccctagcaaagtagacatcaacacagaggatctggaggatggcacatgcagggtcacctactgtcccacagagcctggaaactacattataaacatcaaatttgctgaccagcatgtgcctggtgagtgtggcagccataccccttacctgtggtttggcagcattgctagtcacacacctcagtgcccacctgtaacttgtgcatagtatggattgtacttttttcctacaggcagtcccttttctgtgaaggtgacaggtgagggccgggtgaaagagagtatcacacgcaggcgacgtgccccttctgtggccaatattggcagtcattgtgacctcagcctgaagattcctggtagggaaaagatcaggagggtttctggggcaaccctaagcataagtagagagacagggccctccctgctcaccaagctatcttctatttgtagaaattagcatccaagatatgacagcccaggtgaccagcccatcaggcaagacccatgaggcagagatcgtagaaggagagaaccatacttactgtatccgatttgtgcctgctgagatgggaatgcatacagtcagtgtcaagtacaagggccagcatgtacctgggagccccttccagttcactgtggggcctctgggggaagggggtgctcacaaggtccgtgctggaggccctggcctagagagggctgaagttggagtgccaggtaggtgtacttgtctaattcttgctatcctaggtgggcctaggtgggtgtttgtaggaagtagccagcaggcatgttgatcttggctttgcttagacttggctgaggctgggaaccagtcctctgctccatgagcacacactgctttcccacagcggagttcggcatttggactagggaagctggcgctggaggcctggccattgctgttgaaggccccagcaaggctgagatctctttcgaggaccgaaaggatggctcctgtggtgtggcctacgtagttcaggagccaggtattggaaacagaaatggctggggtagggggaggtaggacctgagtgtgggtatgttgaacccaggtgttacatagtctccacttggttcttattctccatcgtaaggttccagggacaaccaaggggactgtgggagggaggcaagaggcctttcctaggctagcctttggggatatgtggcacctccaggtatagccttatgttctcttcctttccaggtgactatgaggtctcagtcaagttcaacgaggagcacatacctgatagccccttcgtggtgcctgtggcttctccgtctggtgacgcccgccgccttactgtttctagtcttcaggtgaggcactgagagaaactgcccatctggggctcctgggcccggccagaccagagtcaccatagcaagcaggtcttggccctgggctgagtgcctacgcagaaataattctggatggtaggcttctgctctgggaattccgatcatattcaataacccttaatataacttcgtataatgtatgctatacgaagttattaggtctgaagaggagtttacgtccagccaagcttgatatcgagggcccctgcaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctctttatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgaccctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagagcttgcggaacccttaatataacttcgtataatgtatgctatacgaagttattaggtccctcgaagcttatcgattgtggagaacatgtttaggagataggaggttgggggtatgcctgtggcttgctgccctcacagatcaatggctctccctctttaaaccatgttggacaccagatgggtaagtgactctgtcggtcttctgctctcccatttgccctggttctatacatcctgtctaaccacatctgctatgcttccaggagtcagggttaaaggtcaaccagccagcatcttttgcagtcagtctgaatggagccaagggggcaattgatgccaaggtgcacagcccctcaggagctctggaggagtgctatgtcacagagattgaccaaggtgaggctctatccctgcctattcatccaaccatttgacctagctgactagaagtaggctaatgtagtgattcaaaagtgggctcctcagctaaaaaacccttcccagattgacaagcacccaggaagataatggctttccatcatggccactaggaagaacaccttacaccagtgtcatacatgagaaagacataaaaacagagacagagagatagtcaatccctggggctagagaaatggcaattctattgaagcacactagtttcactagtttccctttcccccaagtccatgacctgaagcagattcctccccatctctaagcctgctgctttgtacactgctacagtacactctctaaggaaacagtaaggcaggcattttgcttgttattccaaaagacttacaagtagacttcttaggtattgatagccttagtctcaggatgtatgttattggatgaatacctagaataattctggaagcagagggatctccattgagcagctggattcctgctagtggcaagagaagtgaaggggatgttaaccaattctgttcctttcagatagtattttcaagttgcatacaaaaaagtaccccaaatcccttacctgcatttcagataagtatgctgtgcgtttcatcccacgagagaatggcatctacttgattgatgtcaagttcaatggtactcacattcctggaagtcccttcaagatccgagttggggagcctgggcatggaggggacccaggcttagtgtccgcctatggagcaggcctggaaggtggtgtcacaggtaggtttatgggtacagctctgagtgtagatgtatagccccagcaccaggaagcatgtggaagtatcaaggctgtgctgggtagctgaatgcaaggaagccagtgagatagattctgcctgaccttctattttcccagggagcccagcagagtttattgtgaacacaagcaatgcaggagctggtgccctttcggttaccattgatggcccctccaaggtgaagatggattgccaggagtgccccgagggctatcgtgtcacctatacccccatggcacctggcagctacctcatctccatcaagtatggtggcccctatcacattggtggaagtccctttaaagccaaggtcacaggtgagccttaggctaagctgttggtggccacccaatgccttgatacttgagcagcttcttagtctcacccattctgttttgtaggtcctcgtcttgtaagcaaccacagcctccatgagacatcatctgtgtttgtggactctttgactaaagttgccacggttccccagcatgcaacctcaggcccaggtcctgctgatgtcagcaaggtagtagccaaaggcctggggctaagcaaggcttatgtaggccagaagagcaacttcacagtagattgcagcaaagcaggtgagtagccctatttttagcttctggaagtcgacggtatcgataagcttgatatcgaattcctgcagcccgtaccgaagcgcggtcgagcagtgtggttttcaagaggaagcaaaaagcctctccacccaggcctggaatgtttccacccaatgtcgagcagtgtggttttgcaagaggaagcaaaaagcctctccacccaggcctggaatgtttccacccaatgtcgagcaaaccccgcccagcgtcttgtcattggcgaattcgaacacgcagatgcagtcggggcggcgcggtcccaggtccacttcgcatattaaggtgacgcgaaacggtacggggatccactagt

SEQ ID NO: 49 - DNA, 4032 bp, Artificial Sequence
  Homology directed repair construct for FLNAR mouse
gcgaggtcaagagggagcaaaagtttctgtgaaagagggaaagccaggtgtggtgactgcccatagtcctaatacttgggaggtgtaagtagaagaatcaggggaaccaatcattggctacataatgagttaaagtcattttagcctgtaataaataccctgtgttcagacaaagggagcaaggaagaagattgtgggcccacatcttgttgatagccctatcctgtgcctagggatttccttcgtgcccaaggagacaggggagcacctggtacatgtgaagaagaatggccagcatgtggcaagcagtcccatcccagtagtgatcagccagtcggagataggtgatgccagccgtgtgagggtctctggtcaaggtcttcatgaaggtcatacctttgagcctgcagagtttattattgacaccagagatgcaggtaggcagtgtggtctgctagttggggcaatacagctagctttccttggcattctggcttatagctgctatctatttcccaggctacggtgggcttagtctgtccattgagggccctagcaaagtagacatcaacacagaggatctggaggatggcacatgcagggtcacctactgtcccacagagcctggaaactacattataaacatcaaatttgctgaccagcatgtgcctggtgagtgtggcagccataccccttacctgtggtttggcagcattgctagtcacacacctcagtgcccacctgtaacttgtgcatagtatggattgtacttttttcctacaggcagtcccttttctgtgaaggtgacaggtgagggccgggtgaaagagagtatcacacgcaggcgacgtgccccttctgtggccaatattggcagtcattgtgacctcagcctgaagattcctggtagggaaaagatcaggagggtttctggggcaaccctaagcataagtagagagacagggccctccctgctcaccaagctatcttctatttgtagaaattagcatccaagatatgacagcccaggtgaccagcccatcaggcaagacccatgaggcagagatcgtagaaggagagaaccatacttactgtatccgatttgtgcctgctgagatgggaatgcatacagtcagtgtcaagtacaagggccagcatgtacctgggagccccttccagttcactgtggggcctctgggggaagggggtgctcacaaggtccgtgctggaggccctggcctagagagggctgaagttggagtgccaggtaggtgtacttgtctaattcttgctatcctaggtgggcctaggtgggtgtttgtaggaagtagccagcaggcatgttgatcttggctttgcttagacttggctgaggctgggaaccagtcctctgctccatgagcacacactgctttcccacagcggagttcggcatttggactagggaagctggcgctggaggcctggccattgctgttgaaggccccagcaaggctgagatctctttcgaggaccgaaaggatggctcctgtggtgtggcctacgtagttcaggagccaggtattggaaacagaaatggctggggtagggggaggtaggacctgagtgtgggtatgttgaacccaggtgttacatagtctccacttggttcttattctccatcgtaaggttccagggacaaccaaggggactgtgggagggaggcaagaggcctttcctaggctagcctttggggatatgtggcacctccaggtatagccttatgttctcttcctttccaggtgactatgaggtctcagtcaagttcaacgaggagcacatacctgatagccccttcgtggtgcctgtggcttctccgtctggtgacgcccgccgccttactgtttctagtcttcgggtgaggcactgagagaaactgcccatctggggctcctgggcccggccagaccagagtcaccatagcaagcaggtcttggccctgggctgagtgcctacgcagaaataattctggatggtaggcttctgctctgggattgtggagaacatgtttaggagataggaggttgggggtatgcctgtggcttgctgccctcacagatcaatggctctccctctttaaaccatgttggacaccagatgggtaagtgactctgtcggtcttctgctctcccatttgccctggttctatacatcctgtctaaccacatctgctatgcttccaggagtcagggttaaaggtcaaccagccagcatcttttgcagtcagtctgaatggagccaagggggcaattgatgccaaggtgcacagcccctcaggagctctggaggagtgctatgtcacagagattgaccaaggtgaggctctatccctgcctattcatccaaccatttgacctagctgactagaagtaggctaatgtagtgattcaaaagtgggctcctcagctaaaaaacccttcccagattgacaagcacccaggaagataatggctttccatcatggccactaggaagaacaccttacaccagtgtcatacatgagaaagacataaaaacagagacagagagatagtcaatccctggggctagagaaatggcaattctattgaagcacactagtttcactagtttccctttcccccaagtccatgacctgaagcagattcctccccatctctaagcctgctgctttgtacactgctacagtacactctctaaggaaacagtaaggcaggcattttgcttgttattccaaaagacttacaagtagacttcttaggtattgatagccttagtctcaggatgtatgttattggatgaatacctagaataattctggaagcagagggatctccattgagcagctggattcctgctagtggcaagagaagtgaaggggatgttaaccaattctgttcctttcagatagtattttcaagttgcatacaaaaaagtaccccaaatcccttacctgcatttcagataagtatgctgtgcgtttcatcccacgagagaatggcatctacttgattgatgtcaagttcaatggtactcacattcctggaagtcccttcaagatccgagttggggagcctgggcatggaggggacccaggcttagtgtccgcctatggagcaggcctggaaggtggtgtcacaggtaggtttatgggtacagctctgagtgtagatgtatagccccagcaccaggaagcatgtggaagtatcaaggctgtgctgggtagctgaatgcaaggaagccagtgagatagattctgcctgaccttctattttcccagggagcccagcagagtttattgtgaacacaagcaatgcaggagctggtgccctttcggttaccattgatggcccctccaaggtgaagatggattgccaggagtgccccgagggctatcgtgtcacctatacccccatggcacctggcagctacctcatctccatcaagtatggtggcccctatcacattggtggaagtccctttaaagccaaggtcacaggtgagccttaggctaagctgttggtggccacccaatgccttgatacttgagcagcttcttagtctcacccattctgttttgtaggtcctcgtcttgtaagcaaccacagcctccatgagacatcatctgtgtttgtggactctttgactaaagttgccacggttccccagcatgcaacctcaggcccaggtcctgctgatgtcagcaaggtagtagccaaaggcctggggctaagcaaggcttatgtaggccagaagagcaacttcacagtagattgcagcaaagcaggtgagtagccctatttttagcttctggaagtgcaaagccataaataacaggatggtcagcatgtaggaagtacaggaactgcctttaggggtatgattattgcttttagagctaccttctatttgggccttcgtactttcctttctacccttcaagaaaatgtcctggagtggggctgctgaaaaggtggctcagcagttttcagaggactggggttcaagtcacag

SEQ ID NO: 50 - RNA, 8496 nt, Mus musculus
  Filamin A mRNA, transcript variant X2
cuuggaacagagccuccuuucuaaccaggaaacacuuggcuccccuacacucaucuagccucauaaagguugguccuaaacauaggguuugcagacccugcucuggagugcaaccucuguucugugccucgucguucaugcucggaggugccuacugcuuuaauuaaagggccgucccaucggcgaagcugcagcaccgccccuccguuccucgcgccucaaaaugaguagcucucacucccgcuguggccagagugcggcgguugcgucuccgggaggcaguaucgauucacgggacgcggagaugccggcuaccgaaaaagaccuagcagaagaugcaccguggaagaaaauucagcagaacacauucacccgcuggugcaaugagcaccuuaagugcguaagcaagcgcaucgccaaucugcagacggaccugagcgauggguugcggcucaucgcgcugcucgagguacucagccagaagaaaaugcaccgcaagcacaaccaaagacccacuuuccgccagaugcagcucgaaaaugugucgguggcgcuugaauuccuggaccgugagagcaucaagcucguguccauagacagcaaggcuauuguggauggaaaucugaagcugaucuuaggccucaucuggacccugauccugcacuauuccaucucaaugcccaugugggaugaggaagaggaugaggaggccaagaagcaaacacccaagcagaggcuucuaggcuggauucagaacaagcuaccacagcuucccauuaccaacuucagucgagacuggcagaguggccgggcccugggugcucuuguugauagcugugccccaggccuauguccugacugggacuccugggaugcuaguaagccugugaacaaugcacgggaagccaugcagcaggcugaugacuggcuaggcauuccucaggugauuaccccagaagaaauuguggaccccaauguagaugagcauucuguuaugaccuaccugucucaguuucccaaggccaagcugaagccaggggcuccucuucggcccaaacugaacccgaagaaagcccgagccuaugggccaggcaucgagccuacaggcaauauggugaagaagagagcagaauucacuguggagacccgaagugcuggacagggagaagugcuuguauauguggaggacccagcuggacaccaggaagaggcaaaagugacugccaauaaugacaagaaccguacuuucucugucugguaugucccugaagugacagggacucauaaggugacugugcucuuugcuggccaacauauugccaagagccccuuugagguguauguggacaagucacagggugaugccagcaaagugacugcccagggcccuggucuggagcccaguggcaauauugccaacaagacuaccuacuuugagaucuucacugcaggagcuggcaugggugagguggaaguugucauccaggacccuacaggacagaaaggcacaguggaaccucagcuggaggccaggggugacagcaccuaucgcuguagcuaucagcccaccauggaggguguccauacaguacaugucaccuucgccgguguucccaucccucguagccccuacacugucacuguuggccaagccuguaacccagcugccugccgggcuauugguagaggccuucagcccaaggguguucgagugaaggaaacagccgacuucaagguguacacaaagggcgcuggcaguggggagcuaaaggucacuguaaagggucccaagggugaggagcguguaaagcagaaggacuuaggggaugguguguauggcuuugaauauuacccuacaaucccuggcacauacacugucaccaucacaugggguggccagaacauuggucgaaguccguuugaggugaagguaggcacugaguguggcaaucagaaaguucgggcaugggguccuggccuggaaggaggcauuguuggcaagucagcagacuucguaguagaggccauuggugaugaugugggcaccuuggguuucucuguggaagguccaucacaggcaaagauugaaugugacgacaagggugauggcuccugugaugugcgcuauuggccccaggaggcuggcgaguaugcuguucaugugcuguguaacagugaggauauccgucucaguccuuucauggcugacauccgugaggcaccccaggauuuucacccagacagggugaaggcacgugggccuggauuggagaagacugguguggcugucaacaagccagcagaguucacaguugaugccaagcaugcugggaaggcuccucuucgaguucaaguucaggacaaugagggcugcucuguggaagcgacagucaaggacaauggcaaugguacuuacagcuguucuuaugugcccagaaagccagugaagcacacagccaugguuucuuggggaggugucagcauccccaacaguccuuuccgggugaaugugggagcuggcagccauccaaacaaagucaagguguaugguccaggaguggccaagacugggcucaaggcccaugaaccuaccuacuuuacuguggauuguacugaagcuggccagggagaugucagcauugguaucaagugugccccuggaguagugggccccacugaggcugauauugacuuugauaucauccgcaaugacaaugacaccuucacuguaaaauacacacccuguggggcuggcagcuauaccaucaugguccuuuuugcugaccaggccacacccaccagccccaucagagucaaaguggagccuucucaugaugccagcaaggugaaggcugaggguccuggccuaaaucgcacugguguugaacuuggcaaacccacccauuucacagucaaugcuaaaacugcugggaaaggcaagcuggauguccaauucucaggacuggcuaagggagaugcaguacgggauguggacaucauugaccaccaugauaauaccuacacagucaaguacauuccugugcagcagggcccaguaggugucaaugucacuuauggaggagaucacauccccaagaguccauuuucagugggaguaucuccaagccuggaucucagcaaaaucaaggugucuggccuuggugacaaaguggacguuggcaaagaucaagaguucacaguaaagucaaagggugcagguggucaaggcaaaguagcauccaagauugugagucccucaggugcagcgguacccugcaagguagagccaggccugggagcugacaacagcgugguacguuuugugccccgugaagaggggcccuaugagguggaagugaccuaugauggugugccuguaccuggcagucccuuuccacuagaagcuguggcccccaccaaacccagcaaggugaaggcguuuggaccagggcuacaggggggcaaugcaggcuccccugcccgcuucaccauugauacaaagggugcuggcacugguggccugggccugacaguggaaggccccugugaagcacagcuugagugccuagacaacggggaugguacaugcucugugucuuauguacccacugagccuggggacuacaacaucaacauccuuuuugcugacacccacauuccuggauccccauucaaggcccauguggcuccuuguuuugaugcauccaaggugaagugcucaggcccugggcuggagcgggcuacugcuggugagguagggcaguuccaaguggacuguucaagugcuggcagugcugaguugacgauugagaucugcucugaggcaggacugccagcugaaguauacauucaagaccauggugauggcacacacaccauuaccuauauuccucucuguccuggggcuuacacuguuaccaucaaguauggcggccagccugugcccaacuuccccagcaagcuacagguggaaccugcuguagauaccucagguguacagugcuaugggccugggauugaaggucaaggugucuuccgagaggcaaccacugaguucaguguggaugcccgggcucuuacacagacuggagggccacaugucaaggcucgaguggccaaccccucaggcaaucugacagauaccuaugugcaagacuguggggauggcacauacaaaguggaauacacuccauaugaggaaggaguacacucuguggaugugacuuaugauggcagcccugugcccagcagccccuuccaggugccuguaacagagggcugugaccccucccgggugcguguccauggaccaggcauccaaagugguaccaccaacaaacccaacaaguucacaguagaaaccaggggagcuggcacagguggccugggcuuggcuguugagggucccucagaggccaagaugucuuguauggauaauaaagauggcagcugcucaguagaauacauccccuaugaagcuggaaccuauagccuuaaugucacuuaugguggucaccaagugccagguagucccuucaaggucccuguacaugaugugacagaugcaucuaaagucaaguguucuggaccuggccuaagcccaggcaugguccgugccaaccucccucaguccuuucagguggacacaagcaaagcuggaguugccccacugcaggucaaagugcaggggcccaaaggccugguggagccaguggauguaguggacaaugcugaugguacucagacugucaacuaugugcccagccgagaaggguccuauagcauuucugugcuguauggugaagaagaagugccacggagccccuucaaggucaaggugcugccuacacaugaugccaguaaggugaaggccaguggaccuggacucaacaccacugguguaccugcuagccugccuguggaguucaccauugaugccaaggaugcuggggagggucuguuggcuguccagauuacggauccugaaggcaagcccaagaagacacacauucaagauaaucaugauggcacauacacgguggcuuaugugccagaugugccaggccgguacacaauccucaucaaguaugguggugaugagauucccuuuuccccguaccguguccgggcugugcccacuggggaugccagcaagugcacagucacagugucaaucggaggucacgggcuaggugcuggcauuggccccaccauccagauuggggaggagacggugauuacuguggacacaaaagcagcaggcaaaggcaaagugacuuguacugugugcacaccugauggcucagagguagacguggacgugguggagaaugaggauggcaccuuugacaucuucuacacagcuccccaaccgggcaaauaugucaucugugugcgcuucgguggcgagcaugugcccaacagccccuuccaaguuacagcuuuggcuggggaccaaccaacagugcagaccccauuaaggucucagcagcuggcuccacaguauaacuauccucaggguagccagcaaaccuggauuccagagaggcccauggugggcguuaaugggcuggaugugaccagccugaggcccuuugaucuugucauccccuucacuaucaagaagggggagaucacuggggaaguucgaaugcccucaggcaagguggcccagccuuccauuacugauaacaaagauggcacuguuacuguacguuacucacccagugaagcuggccugcaugaaauggacauucgcuaugacaauaugcauaucccaggaagcccucugcaguucuauguugauuaugucaacuguggccacaucacugcuuaugguccuggccuuacccauggaguggucaacaaaccugccaccuucacugucaacaccaaggaugcaggagaggggggcuugucucuggccauugaggguccaucuaaagcagaaaucaguugcacugacaaccaggauggaacaugcagugucucuuaccugccuguacugccuggugacuauagcauccuaguuaaguacaaugaucaacacaucccaggcagucccuuuacugccagaguaacaggugacgauuccaugcguaugucccaccuaaagguggguucugcugcugauauccccaucaauaucucagaaacagaccuuagccuacucacagccacuguggugccaccuucgggucgagaggaacccugucugcugaaacguuugcgaaauggccacguggggauuuccuucgugcccaaggagacaggggagcaccugguacaugugaagaagaauggccagcauguggcaagcagucccaucccaguagugaucagccagucggagauaggugaugccagccgugugagggucucuggucaaggucuucaugaaggucauaccuuugagccugcagaguuuauuauugacaccagagaugcaggcuacggugggcuuagucuguccauugagggcccuagcaaaguagacaucaacacagaggaucuggaggauggcacaugcagggucaccuacugucccacagagccuggaaacuacauuauaaacaucaaauuugcugaccagcaugugccuggcagucccuuuucugugaaggugacaggugagggccgggugaaagagaguaucacacgcaggcgacgugccccuucuguggccaauauuggcagucauugugaccucagccugaagauuccugaaauuagcauccaagauaugacagcccaggugaccagcccaucaggcaagacccaugaggcagagaucguagaaggagagaaccauacuuacuguauccgauuugugccugcugagaugggaaugcauacagucagugucaaguacaagggccagcauguaccugggagccccuuccaguucacuguggggccucugggggaagggggugcucacaagguccgugcuggaggcccuggccuagagagggcugaaguuggagugccagcggaguucggcauuuggacuagggaagcuggcgcuggaggccuggccauugcuguugaaggccccagcaaggcugagaucucuuucgaggaccgaaaggauggcuccugugguguggccuacguaguucaggagccaggugacuaugaggucucagucaaguucaacgaggagcacauaccugauagccccuucguggugccuguggcuucuccgucuggugacgcccgccgccuuacuguuucuagucuucaggagucaggguuaaaggucaaccagccagcaucuuuugcagucagucugaauggagccaagggggcaauugaugccaaggugcacagccccucaggagcucuggaggagugcuaugucacagagauugaccaagauaaguaugcugugcguuucaucccacgagagaauggcaucuacuugauugaugucaaguucaaugguacucacauuccuggaagucccuucaagauccgaguuggggagccugggcauggaggggacccaggcuuaguguccgccuauggagcaggccuggaagguggugucacagggagcccagcagaguuuauugugaacacaagcaaugcaggagcuggugcccuuucgguuaccauugauggccccuccaaggugaagauggauugccaggagugccccgagggcuaucgugucaccuauacccccauggcaccuggcagcuaccucaucuccaucaaguaugguggccccuaucacauugguggaagucccuuuaaagccaaggucacagguccucgucuuguaagcaaccacagccuccaugagacaucaucuguguuuguggacucuuugacuaaaguugccacgguuccccagcaugcaaccucaggcccagguccugcugaugucagcaagguaguagccaaaggccuggggcuaagcaaggcuuauguaggccagaagagcaacuucacaguagauugcagcaaagcagguaacaacaugcugcuggugggcgugcauggcccaaggacacccugugaagagauccuggugaaacacaugggcagccgccucuauagugucucuuaccugcucaaggacaaaggggaguacacacugguggucaaguggggugaugagcauaucccaggcagcccauaccgcauuaugguacccugagccugccaccuaaguuggcaccugugccagcuagaagcucccauggcaaugagcauccccacaccuguccuauucccaagaagccccauuuuccuuuccugagcccuggacccucccuucccuggaucacucuggccgcucuucacugcauucgcccuugcccugcacuguguucaccugucucugggcuuucacuugggcagagggagccauuugguggcagagcaugucuucuuugguucugggagguggaaguccuauguacaaccaccuucuaguucucuuucccagccaagaggaauaaaauuuugcuuccguuguccugugaa

SEQ ID NO: 51 - RNA, 50 nt, Artificial Sequence
  Synthetic oligonucleotide
aaagaugcuggcugguugaccuuuaacccugacucccgaagacuagaaac

SEQ ID NO: 52 - DNA, 21 bp, Artificial Sequence
  primer
gtcaagttcaacgaggagcac

SEQ ID NO: 53 - DNA, 21 bp, Artificial Sequence
  primer
gtgcaccttggcatcaattgc

SEQ ID NO: 54 - DNA, 658 bp, Artificial Sequence
  modified mouse DNA
ctttcctaggctagcctttggggatatgtggcacctccaggtatagccttatgttctcttcctttccaggtgactatgaggtctcagtcaagttcaacgaggagcacatacctgatagccccttcgtggtgcctgtggcttctccgtctggtgacgcccgccgccttactgtttctagtcttcgggtgaggcactgagagaaactgcccatctggggctcctgggcccggccagaccagagtcaccatagcaagcaggtcttggccctgggctgagtgcctacgcagaaataattctggatggtaggcttctgctctgggattgtggagaacatgtttaggagataggaggttgggggtatgcctgtggcttgctgccctcacagatcaatggctctccctctttaaaccatgttggacaccagatgggtaagtgactctgtcggtcttctgctctcccatttgccctggttctatacatcctgtctaaccacatctgctatgcttccaggagtcagggttaaaggtcaaccagccagcatcttttgcagtcagtctgaatggagccaagggggcaattgatgccaaggtgcacagcccctcaggagctctggaggagtgctatgtcacagagattgaccaaggtgaggctctatccc

SEQ ID NO: 55 - DNA, 26 bp, Artificial Sequence
  part of mouse Filamin A cDNA
ctagtcttcaggagtcagggttaaag

SEQ ID NO: 56 - RNA, 50 nt, Artificial Sequence
  Synthetic oligonucleotide
aaagaggcuggcugguugaccuuuagcccugacucccgaaggcuagaaac